(19) United States
(12) Patent Application Publication
(10) Pub. No.: US 2005/0095648 A1
(43) Pub. Date: May 5, 2005

(54) Method for designing linear epitopes and algorithm therefor and polypeptide epitopes

(76) Inventors: Mario Geysen, Charlottesville, VA (US); Dana Ault-Riche, San Diego, CA (US)

Correspondence Address: Stephanie Seidman;FISH & RICHARDSON P.C., 12390 El Camino Real, San Diego, CA, 92130, US

(21) Appl. No.: 10/806,924

(22) Filed: Mar. 22, 2004

Related U.S. Application Data
(63) Continuation-in-part of application No. PCT/US03/34821, filed on Oct. 30, 2003.
(63) Continuation-in-part of application No. 10/699,088, filed on Oct. 30, 2003.

(51) Int. Cl. C12Q 1/68; G01N 33/53; G01N 33/543

(52) U.S. Cl. 435/7.1; 436/518; 530/350

(57) ABSTRACT

Collections of highly antigenic highly specific polypeptides are provided herein. Also provided are methods of generating collections of highly antigenic highly specific polypeptides and methods of synthesizing such collections.

### RELATED APPLICATIONS

This application is a continuation-in-part of International PCT Application Serial No. PCT/US03/34821 filed Oct. 30, 2003, to Dana Ault-Riche, H. Mario Geysen and Bruce Atkinson, entitled “METHODS FOR PRODUCING POLYPEPTIDE-TAGGED COLLECTIONS AND CAPTURE SYSTEMS CONTAINING THE TAGGED POLYPEPTIDES.” This application also is a continuation-in-part of U.S. application Ser. No. 10/699,088 filed Oct. 30, 2003, to Dana Ault-Riche and Bruce Atkinson, entitled “METHODS FOR PRODUCING POLYPEPTIDE-TAGGED COLLECTIONS AND CAPTURE SYSTEMS CONTAINING THE TAGGED POLYPEPTIDES.”

This application is related to U.S. application Ser. No. 10/699,114, and International PCT Application Serial No. PCT/US03/34693, each entitled “SYSTEMS FOR CAPTURE AND ANALYSIS OF BIOLOGICAL PARTICLES AND METHODS USING THE SYSTEMS”, and to U.S. application Ser. No. 10/699,113 and International PCT Application Serial No. PCT/US03/34747, each entitled, “SELF-ASSEMBLING ARRAYS AND USES THEREOF”, each filed Oct. 30, 2003.

The subject matter of each of the above-noted applications, provisional applications, published applications and international applications is incorporated in its entirety by reference thereto.

### FIELD OF INVENTION

Methods for generating collections of highly antigenic highly specific polypeptide sequences are provided. The highly antigenic highly specific polypeptides can be used binding partners for use with capture agents which recognize the highly antigenic highly specific polypeptides.

### BACKGROUND

In the process of drug development, a new drug candidate is often selected from a large collection of molecules, referred to as a molecular library. Methods in chemistry, such as combinatorial chemistry, permit generation of large molecular libraries. For example, molecular libraries containing over a million different chemical species can be created using combinatorial methods. The resulting libraries can be screened using high throughput technologies. High throughput screening technologies are designed to allow rapid testing of molecules in molecular libraries for drug-like properties. High throughput screening technologies can use robotics and engineering principles to empirically test molecules in a molecular library.

Most molecular libraries are created so that different molecular species within the library are spatially isolated from each other. An alternative to spatially separated libraries is to encode the library with unique identifier tags. For example, molecular libraries can be encoded with unique identifier mass tags that can be read using a mass spectrometer. The unique mass signature associated with the mass tag can be used to identify the structure of the encoded molecule. Molecular libraries also can be encoded with optical bar codes that can be read using an appropriate optical reading device, such as a fluorescence activated cell sorter (FACS) device.

Molecular libraries also can be encoded with linear peptide epitopes. In this case the epitope tag is identified by binding to an epitope-specific capture agent, such as an antibody, which is located at a pre-determined site within an array, or is in turn encoded. Central to this strategy is the ability to create a collection of capture agents that specifically bind to a correlated collection of linear epitopes.

Although the above methods exist, there is a need methods for rapid and economical testing of large molecular libraries so that better candidate drug molecules can be discovered. Also there remains a need for new methods and tools to design linear epitopes that can be specifically and tightly bound by capture agents. Therefore, among the objects herein, it is an object to provide such methods and products.

### SUMMARY

Provided herein are collections of polypeptides and methods for generating collections thereof. The methods for generating collections of polypeptides include selecting subsets of polypeptides from the total number of possible polypeptides. The subsets can be limited by scale and/or by biasing the collection towards one or more selected properties. Subsets can be limited by imposing a set of criteria, for example, by selecting a polypeptide length or range of lengths, by choosing a subset of polypeptides which are more similar or dissimilar to each other, by constraining the number of amino acids selected to construct polypeptides of the subset, and/or by constraining particular positions of polypeptides in the subset. Subsets also can be limited by imposing criteria for a selected property, for example, by selecting polypeptides that have a higher probability of being antigenic in a particular host, and/or have reduced antigenicity in a second host. Selection criteria also can include criteria based on the ease of and success rate of synthesis or high yield of polypeptides, stability, solubility and any other properties desired. Collections of polypeptides provided herein include collections constrained by any or all of these properties. Such collections include binding partner polypeptides that specifically bind to capture agents. Provided herein are methods of synthesis for collections of polypeptides. Also provided herein are methods of employing collections of polypeptides as tags in molecular synthesis and sorting.

Provided herein are collections of antigenic polypeptides. In one embodiment, a collection of antigenic polypeptides contains at least three antigenic polypeptides of 5 to 8 unique residues and includes at least 4 residues, designated critical residues. The critical residues can be selected from a list of ranked amino acids. In one aspect of the embodiment, the amino acids are selected from E, P, Q, N, F, H, T, K, L, D. Critical residues occupy the N and C terminal positions in each polypeptide and no more than three polypeptides in the collection contain the same four critical residues.

In another embodiment, the collection of polypeptides contain at least two polypeptides that contain the same four critical residues but each of the two polypeptides has non-critical residues at different positions. The polypeptides contain at least 6 unique residues and at least 2 non-critical residues that are adjacent to each other. In one example, the non-critical residues are selected from among Y, S and G.

The collections include polypeptides of 4, 5, 6, 7, and 8 unique residues. The polypeptides of the collection can be at least 6, 7, 8, 9, 10, 11, 1 2, 1 5, 20, 25, 30, 35, 40, 45 amino acids in length. The collections can contain at least 4, 5, 6, 7, 8, 9, 10, 20, 25, 30, 40, 50, 60, 70, 80, 90 or 100 members. The collections can also contain at least 200, 300, 400, 500, 750, 1000, 5000, or 10,000 members.

The collections of polypeptides include polypeptides that are antigenic, for example antigenic in a non-human subject, such as a rodent or bird. In one embodiment, the polypeptides of the collection exhibit higher antigenicity in a non-human subject than in a human subject.

Provided herein are exemplary collections of binding partner polypeptides, including collections of 2, 3, 4, 5, 6, 7, 8, 9, 10, 15, 20, 50, 100 or more of polypeptides of any of SEQ ID Nos. 1-911. Also provided herein are polypeptides and collections of polypeptides containing any of the polypeptides of any of SEQ ID Nos. 1-911. Also provided are fusion proteins containing a first polypeptide or one or more amino acids conjugated to any of the polypeptides set forth as SEQ ID Nos. 1-911. The fusion proteins can be at least 6, 7, 8, 9, 10, 11, 12, 15, 20, 25, 30, 35, 40, 45 amino acids in length.

The collections provided herein include addressable collections. For example, polypeptides of the collection can be positionally addressable. The polypeptides of the collection can also be immobilized on a solid support, such as by direct or indirect linkage via a linker to the solid support.

The collections of polypeptides can be conjugated to other molecules. In one example, collections of polypeptides are conjugated molecules selected from polypeptides, nucleic acids and small organic molecules. In another example, the polypeptides of the collection are conjugated to members of a library. For example, they can be conjugated to members of a nucleic acid library, a polypeptide library, a natural products library and a combinatorial chemistry library.

Also provided herein collections of capture agent—binding partner polypeptide pairs containing a collection of polypeptides that can are antigenic, such as described herein for use as binding partners and a collection of capture agents. Each capture agent in the collectino binds to a binding partner polypeptide within the collection of binding partner polypeptides. In one example, the capture agents of the collection are antibodies or antibody fragments. The collection of binding partner polypeptides can include 2, 3, 4, 5, 6, 7, 8, 9, 10, 15, 20, 50, 100 or more of polypeptides of any of SEQ ID Nos. 1-911. The collections typically include at least 10 members, including at least two of the polypeptides of any of SEQ ID Nos 1-911.

Also provided herein are methods of generating highly antigenic highly specific binding polypeptides. The methods include the steps of ranking amino acids based upon pre-determined criteria for antigenicity, where n amino acids are ranked and based upon the ranking using the top m to n-1, generating all combinations of the amino acids in a polypeptide of pre-selected length m residues to produce a set S1 of polypeptides of length m residues. From that set, based upon pre-determined criteria for dissimilarity, a subset of dissimilar polypeptides is selected. In such methods, n is the number of amino acids in a preselected set of possible amino acids; m is a length of amino acids pre-selected to have a minimum length sufficient affinity to bind to a selected capture agent up to a length that retains specific binding to a selected capture agent.

In the methods, any number and type of amino acids can be ranked and selected. For example, the number of amino acids can be chosen where n is equal to 5, 6, 7, 8, 9, 10, 11, 12, 13, 14, 15, 16, 17, 18 or 19; n can also be between 20 and 10,000. The number of amino acids chosen for the length of polypeptides m is equal to 4, 5, 6, 7, 8, 9, 10, 11, 12, 13, 14, 15, 16, 17, 18, 19, 20, 21, 22, 23, 24, 25, 26, 27, 28, 29 or 30. The methods also include generating polypeptides where m is an integer between 4 and 6 or 4 and 7 or 4 and 8 or 4 and 12 or 5 and 7 or 5 and 8 or 5 and 12 or 6 and 8 or 6 and 10 or 6 and 12 or 8 and 12.

The methods include generating polypeptides with naturally-occurring amino acids, non-naturally occurring amino acids and combinations of non-naturally occurring and naturally-occurring amino acids. The methods include selecting amino acids based on predetermined criteria for antigenicity. In one example of the methods, the pre-determined criteria for antigenicity is based upon frequency of the amino acids in a pre-selected set of antigenic polypeptides. In one embodiment, the amino acids selected are selected from among E, P, Q, N, F, H, T, K, L, D, S, G and Y.

The methods provided herein further include generating highly antigenic highly specific polypeptides containing critical and non critical amino acids. In one embodiment, the methods include generating a subset of polypeptides of length q residues, wherein q=m+r and r is the number of non-critical amino acids, wherein r is an integer equal to or greater than 1 and q is an integer greater than 4. In another embodiment, the N and C terminal amino acids of the polypeptides of length q residue are critical amino acids. In yet another embodiment at least 2 of the non-critical amino acids are adjacent in the polypeptide. The methods include generating polypeptides with any number of non-critical amino acids. For example, the number of non-critical amino acids r can be 1, 2, 3, 4, 5, 6, 7, 8, 9 or 10. The methods include generating highly antigenic highly specific polypeptides of any length. for example, the length of such polypeptides include polypeptides of length q, where q is an integer between 5 and 100 or 5 and 50 or 5 and 30 or 5 and 20 or 5 and 10.

The methods for generating highly antigenic highly specific polypeptides provided herein also include selecting a subset of dissimilar polypeptides. For example, dissimilarity refers to functional and structural dissimilarity based upon predetermined criteria. Dissimilarity is assessed by comparing each polypeptide in the set S1 an arbitrarily selected reference polypeptide from the set S1 by comparing corresponding critical residue based upon position in the polypeptides. Polypeptides from set S1 are selected that contain residues most dissimilar from the reference polypeptide. In one embodiment, dissimilarity is determined by calculating a similarity score from a similarity matrix by comparing values for the corresponding critical residues in the reference polypeptide to the corresponding critical residues in the polypeptides of set S1, combining the scores for the residues in each polypeptide to generate a score for each polypeptide and selecting those below a predetermined score.

Also provided herein are collections of binding partner polypeptides, comprising 2, 3, 4, 5, 6, 7, 8, 9, 10, 15, 20, 50, 100or more polypeptides generated by methods of producing highly antigenic highly specific polypeptides. Also provided herein are collections of capture agent—binding partner polypeptide pairs comprising collections of binding partner polypeptides generated by the methods herein and collections of capture agents. In such collections, the capture agents each bind to a binding partner polypeptide within the collection of binding partner polypeptides. The collections can be contained in kits that optionally including instructions preparing capture agents that specifically bind to members of the collection.

Also provided herein are methods for synthesizing an addressable collection of polypeptides. The methods include providing a collection of b tags and a collection of b addressable capture agents, where each capture agent binds a unique tag and b is the number of tag-capture agent pairs. The tags are presented in an addressable format suitable for peptide synthesis and a collection of polypeptides is synthesized on the collection of tags such that each tag is conjugated directly or indirectly via a linker to a synthesized polypeptide. Each of the synthesized polypeptides comprises a number of variable amino acid positions v and optionally a number n of fixed amino acid positions each designated N; each N can be the same or different amino acids. In the method of synthesis, a subset of v positions is synthesized in a first round of synthesis to generate a collection of tag-v₁ polypeptides such that each unique tag is directly or indirectly linked to an amino acid and each tag has a unique combination of amino acids at the synthesized variable positions. The collection of synthesized tag-v₁ polypeptides is mixed and the collection of tag-v₁ polypeptides is split into b addressable first-round subsets. Each first-round subset contains a collection of tag-v₁ polypeptides representing on average every possible combination of amino acids at the synthesized variable positions. A further subset of variable positions v₂ is synthesized in a further round of synthesis, such that each tag-v₁ polypeptide is conjugated to a unique combination of amino acids at v₂ positions to generate b subsets of tag-v₁v₂ polypeptides. The resulting subsets of tag-v₁v₂ polypeptides are contacted with the addressable collection of b capture agents to produce an addressable collection of synthesized polypeptides. In one example, the number of variable positions synthesized v is 4 and the subset of variable positions synthesized in the first round is equal to 2.

The collection of capture agents can be conjugated to a solid support. In one example, b collections of b capture agents are conjugated to a solid support. In another example, the capture agents are antibodies or antibody fragments. In another embodiment, the method further includes incubating the addressable collection of synthesized polypeptides or a subset thereof with one or more collections of molecules under conditions where one or more molecules specifically binds to the synthesized polypeptides. In one example, the collections of molecules include molecules selected from antibodies, fragments of antibodies and polypeptides.

The methods include optionally synthesizing polypeptides of length d, containing n fixed amino acids positions, where the polypeptides of the collection have the same amino acid at a fixed position. The number of fixed positions can be 0, 1, 2, 3, 4, 5, 6, 7, 8, 9, 10, 11, or 12. The number of variable positions included for synthesis can be 2, 3, 4, 5, 6, 7 or 8. In one embodiment, the synthesized polypeptides are highly antigenic highly specific polypeptides, such as sequences of highly antigenic highly specific polypeptides generated by the methods provided herein.

Also provided herein are methods for synthesizing an addressable collection of molecules. The methods include providing a collection of b tags and a collection of b addressable capture agents, where each capture agent binds a unique tag and b is the number of tag-capture agent pairs. The tags are presented in an addressable format suitable for chemical synthesis. A collection of molecules is synthesized such on starting molecules and each tag is conjugated directly or indirectly via a linker to a starting molecule. Each synthesized molecule contains a number of variable constituent positions X conjugated to the starting molecule. The method of synthesis includes synthesizing a subset of X positions X₁ in a first round of synthesis to generate a collection of tag-X₁ molecules, whereby each unique tag is conjugated to a unique combination of constituents at the synthesized X₁ positions. The collection of synthesized tag-X₁ molecules is mixed and split into b addressable first-round subsets,such that each first-round subset contains a collection of tag-X₁ molecules representing on average every possible combination of constituents at the synthesized X₁ positions. A further subset of constituent positions X₂ is synthesized in a further round of synthesis, such that each first-round subset is conjugated to a unique combination of constituents at X₂ positions to generate b second-round subsets. The resulting tag-X₁X₂ is contacted with an addressable collection of b capture agents to produce an addressable collection of synthesized molecules. In one example, the synthesized molecules are selected from among nucleic acid molecules, polymers, biopolymers, polypeptides, and small organic molecules. In one example, the starting molecule is a pharmacophore. In another example, the starting molecule is a monomer and the synthesized molecules are polymers. In another example, the tags are highly antigenic highly specific polypeptides. In one example, the tags comprise any of the sequences set forth in SEQ ID NOs. 1-911. In yet another example, the capture agents are antibodies or antibody fragments.

### BRIEF DESCRIPTION OF THE FIGURES

FIG. 1 depicts an example of a suitable computer system that can implement the algorithms described herein to generate collections of polypeptide sequences.

FIG. 2 shows one embodiment of the operations that are performed with the computer system of FIG. 1 to generate collections of polypeptide sequences.

FIGS. 3A and 3B show an embodiment of synthesis methods for collections of polypeptides.

DETAILED DESCRIPTION
OUTLINE

| A. | Definitions | |
|---|---|---|
| B. | Identification of Highly Antigenic Highly Specific Polypeptides | |
| | 1. | HAHS polypeptides and collections thereof |
| | 2. | Description of the methods |

| | |
|---|---|
| a. | Selecting amino acids |

| | |
|---|---|
| i. | Ranking antigenicity |
| ii. | Generating sequences with chosen amino acids |
| iii. | Use of non-naturally occurring amino acids |

| | |
|---|---|
| b. | Biased subsets of polypeptides |
| c. | Critical and non-critical amino acids |
| d. | Selecting a dissimilar set |
| e. | Limiting the amino acids chosen for non-critical |
| | positions |

| | 3. | Production of HAHS polypeptides |
|---|---|---|
| | | Methods for preparing collections of HAHS polypeptides in |
| | | an addressable format |
| | 4. | Assessment of antigenicity |
| C. | Identification of capture agents which bind HAHS polypeptides | |
| | 1. | Raising antibodies |
| | 2. | Antibody Library Screening |
| | 3. | Engineered Capture Agents |
| D. | Producing molecules tagged with HAHS polypeptide binding | |
| | partners | |
| | 1. | Chemical conjugates |
| | 2. | Fusion proteins |
| | 3. | Linkers |
| | 4. | Tagged libraries |
| E. | Use of binding proteins in capture systems | |
| | 1. | Preparation of Capture Systems |

| | |
|---|---|
| a. | Preparation of binding partners |
| b. | Capture agents |

| | |
|---|---|
| 2. | Preparation of capture agent arrays |

| | |
|---|---|
| a. | Immobilization and activation |
| b. | Stabilization of capture agents and polypeptide binding |
| | partners |

| | |
|---|---|
| 3. | Screening |
| 4. | Combinatorial synthesis of tagged libraries |

| | |
|---|---|
| F. | Kits |
| G. | Software |
| H. | Diagnostics |
| I. | EXAMPLES |

### A. DEFINITIONS

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, an highly antigenic, highly specific polypeptide (also referred to herein as HAHS polypeptides) is a polypeptide that specifically binds to a unique member of a collection of capture agents (i.e. binds with at least 1-, 2-, 5-10-fold or greater affinity to one unique member compared to all other members in a collection of at least 3, 5, 10, 50, 100 or more unique members). Collections of HAHS polypeptides are collections of polypeptides that specifically bind capture agents such that in collections thereof each HAHS polyeptide in the collection will bind to a unique member of a collection of capture agents with greater affinity (tyically at least 1, 2, 5, 10-fold or more) than to any other member of the collection of capture agents. The collections of capture agents include at least 3, 5, 10, 50, 100 or more unique capture agents.

The HAHS polypeptides are antigenic in that capture agents that specifically bind HAHS polypeptides are readily designed or prepared. Hence, antigenic refers the ability of the HAHS polypeptides to bind to capture agents with high affinity and specificity. For example, an HAHS polypeptide specifically binds to a capture agent such as an antibody or any fragment of an antibody of sufficient length to bind to an epitope. The HAHS polypeptides that result from the methods herein can be used to generate capture agents, such as by immunization of animals, particularly rodents and birds, or by in vitro screening methods, such as phage display or other such methods. Thus, for example, HAHS polypeptides can be prepared from application of methods herein to generate collections of polypeptides that specifically bind capture agents such as antibodies, antibody fragments and engineered molecules that contain binding regions of antibodies and antibody fragments. HAHS polypeptides also can be generated and/or selected to be antigenic in one host and less antigenic in another host. For example, HAHS polypeptides can be highly antigenic in mice but less antigenic or non-antigenic in humans.

As used herein, antigenic when used in the context of highly antigenic highly specific polypeptides refers to polypeptides that induce, upon administration to a host, antibodies that are specific for the HAHS polypeptides or upon screening, or select for (such in display or panning methods) capture agents, such as antibodies or antibody fragments, with specific and selective binding to the HAHS polypeptides.

As used herein, a molecule, such as capture agent, that specifically binds to a polypeptide, such as a HAHS polypeptide provided herein, typically has a binding affinity (Kₐ) of at least about 10⁶ I/mol, 10⁷ I/mol, 10⁸ I/mol, 10⁹ I/mol, 10¹⁰ I/mol or greater (generally 10⁸ or greater) and binds generally with greater affinity (typically at least 10-fold, generally 100-fold or) than to the molecules and biological particles that are to be detected or assessed in the methods that employ the capture systems. Thus, affinity refers to the strength of interaction between two or more molecules, such as a capture agent and a HAHS polypeptide binding partner. Typically, an HAHS polypeptide specifically binds to a unique capture agent in collection with at least 1-, 2-, 5-10-fold or greater affinity than to all others capture agents in a collection.

As used herein, specificity (or selectively) with respect to binding partners and capture agents refers to the greater affinity a binding partner and a capture agent exhibit for each other compared to their affinities for other molecules and biological particles.

As used herein, a binding partner generically refers to a polypeptide that includes a sequence of amino acids, that specifically binds to a capture agent, such as an HAHS polypeptide. Binding partners can contain HAHS polypeptides and optionally additional sequences such as, but not limited to, a specific amplification sequence (herein referred to as an R-tag) and additional domains, such as a detectable label, for example fluorescent or enzymatic polypeptide, and a ligand-binding domain. Further, binding partners can include non-polypeptide moieties, such as but not limited to, a radiolabel.

As used herein, a capture agent refers to a molecule that has an affinity for a given ligand or with a defined sequence of amino acids. Capture agents can be naturally-occurring or synthetic molecules, and include any molecule, including nucleic acids, small organics, proteins and complexes that specifically bind to specific sequences of amino acids. Capture agents can be used in their unaltered state or as aggregates with other species. They can be attached or in physical contact with, covalently or noncovalently, a binding member, either directly or indirectly via a specific binding substance or linker. Contemplated herein are capture agents which bind highly antigenic, highly specific polypeptides. Exemplary capture agents which bind HAHS polypeptides include, but are not limited to, antibodies and antibody fragments, monoclonal antibodies and antisera reactive or isolated components thereof, engineered and synthetically designed antibodies, and polypeptides which contain one or more antigen binding regions such as variable regions and complementarity determining regions. Other examples of capture agents are set forth throughout the disclosure.

Capture agents and binding partners are pairs of molecules, generally proteins that specifically bind to each other. One member of the pair is a polypeptide binding partner that can be conjugated to another molecule or particle; the other member is anything that specifically binds thereto. Collections of capture agents, such as antibodies or portions thereof and mixtures thereof, specifically bind to known or knowable defined sequences of amino acids that are generally at least about 2 to 100 amino acids in length, typically 2-20 amino acids in length, such as 2, 3, 4, 5, 6, 7, 8, 9, 10, 12, 14, 16, 18, or 20 amino acids in length.

HAHS polypeptides can be used as binding partners with capture agents that bind them. Hence, methods as described herein readily produce pairs of HAHS polypeptides and capture agents which bind them. As described herein, HAHS polypeptides can be designed such that there is little detectable cross-reactivity, such as by ELISA assay, between or among different pairs of HAHS polypeptides and capture agents in a collection. Each HAHS polypeptide is selective for a capture agent such that the capture agent and HAHS polypeptide bind to each other with a greater affinity for each other than for another HAHS polypeptide or capture agent in a collection of HAHS polypeptides and capture agents. Generally, an HAHS polypeptide and capture agent bind to each other with an affinity that is about 10-fold, 100-fold or greater than any affinity they have for other HAHS polypeptides or capture agents in the collection.

Polypeptide binding partners and HAHS polypeptides can be encoded by nucleic acid molecules. Optionally, such nucleic acid molecules can include additional sequences of nucleotides that can serve as primers or portions of primers, for example primers useful for introducing sequences of HAHS and binding partner sequences into other nucleic acid molecules. Other optional sequences can include other functional signals, such as stop codons, or ribosome binding sites, translation initiation sites and other such sites. The domains can be adjacent to each other or separated or overlapping. In some embodiments, these optional sequences are referred to herein as an R-tag.

As used herein, antigenic ranking refers to a statistical probability that an amino acid or set thereof occurs in an antigenic polypeptide, including epitopes in naturally occurring polypeptides.

As used herein, a similarity ranking refers to a comparison among amino acids and is represented or determined as a probability or fraction that two amino acids are structurally and/or functionally similar. For example, two identical amino acids have a similarity ranking of 100; two very dissimilar amino acids, such as proline and tyrosine have a ranking of 0.

As used herein, a subset of a set contains at least one less member than the set.

As used herein, a critical residue or amino acid in an HAHS polypeptide is one that influences the affinity or specificity of binding to the binding protein (capture agent). Critical residues taken from the set of naturally occurring amino acids can only be replaced by a subset of amino acids (usually 1 or 2 amino acids) or in some cases, can not be replaced by any other amino acid from this set.

As used herein, a non-critical residue or amino acid in an HAHS polypeptide is one that does not influence the affinity or specificity of binding to the binding protein (capture agent). Noncritical residues can be replaced by a larger subset of amino acids (for example, when taken from the set of naturally occurring amino acids, they can be replaced usually 10 or more amino acids or in some cases, by any other amino acid from this set) without affecting the affinity or specificity of binding. In some cases, non-critical residues are used to confer additional functionalities or properties on polypeptides. In this case, they can typically only be replaced by a limited number of amino acids to retain the functionality or property.

As used herein, an amino acid is an organic compound containing an amino group and a carboxylic acid group. A polypeptide comprises two or more amino acids. For purposes herein, amino acids include the twenty naturally-occurring amino acids non-natural amino acids, and amino acid analogs. These include amino acids wherein α-carbon has a side chain.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

As used herein, naturally occurring amino acids refers to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include amino acids or analogs of amino acids other than the 20 naturally occurring amino acids and include, but are not limited to, the D-isostereomers of amino acids. Exemplary non-natural amino acids are described herein and are known to those of skill in the art.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726). Each naturally occurring L-amino acid is identified by the standard three letter code (or single letter code) or the standard three letter code (or single letter code) with the prefix “L-”; the prefix “D-”indicates that the stereoisomeric form of the amino acid is D.

As used herein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p.224).

Such substitutions can be made in accordance with those set forth in TABLE 1 as follows:

TABLE 1

| Original | Conservative |
|---|---|
| residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | The |
| The (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, the term “polypeptide” is used interchangeably with the term “protein” and includes peptides containing two or more amino acids. A polypeptide can be a single polypeptide chain, or to two or more polypeptide chains that are held together by non-covalent forces, by disulfide cross-links, or by other linkers (e.g. peptide linkers). Thus, a single heavy or light chain of an antibody, or an antibody fragment containing all or part of the heavy and light chains of an antibody, no matter how the chains are associated or joined, are exemplary molecules that are included within the term “a polypeptide.” A polypeptide can contain non-proteinaceous components, such as sugars, lipids, detectable labels or therapeutic moieties. A polypeptide can be derivatized by chemical or enzymatic modifications (e.g. by replacement of hydrogen by an alkyl, acyl, or amino group; esterification of a carboxyl group with a suitable alkyl or aryl moiety; alkylation of a hydroxyl group to form an ether derivative; phosphorylation or dephosphorylation of a serine, threonine or tyrosine residue; or N- or O-linked glycosylation) or can contain substitutions of an L-configuration amino acid with a D-configuration counterpart.

As used herein, unique, in reference to amino acids within a polypeptide means that there is no duplication or any multiples of a particular amino acid within the polypeptide length.

As used herein, a three-dimensional structure refers to the physical structure of a molecule or biological particle.

As used herein, an address refers to a unique identifier whereby an addressed entity can be identified. An addressed moiety is one that can be identified by virtue of its address. Addressing can be effected by position on a surface or by other identifiers, such as a tag encoded with a bar code or other symbology, a chemical tag, an electronic, such RF tag, a color-coded tag or other such identifier.

As used herein, a capture system refers to an addressable collection of capture agents and binding partner-tagged molecules bound thereto, where each different binding partner specifically binds to a different capture agent.

As used herein, a landscape is the information produced or presented on a canvas or array.

As used herein, an addressable collection of capture agents is a collection of protein agents, such as antibodies, that specifically bind to pre-selected binding partners that contain sequences of amino acids, in which each member of the collection is labeled and/or is positionally located to permit identification of the capture agent and binding partner. The addressable collection is typically an array or other encoded collection in which each locus contains capture agents, such as antibodies, of a single specificity and is identifiable. The collection can be in the liquid phase if other discrete identifiers, such as chemical, electronic, colored, fluorescent or other tags are included. Capture agents, include antibodies and other anti-tag receptors. Any moiety, such as a protein, nucleic acid or other such moiety, that specifically binds to a pre-determined sequence of amino acids is contemplated for use as a capture agent.

As used herein, an addressable collection of binding sites refers to the resulting sites produced upon binding of the capture agents to binding partner-tagged reagents. Each capture agent sorts reagents (such as molecules and biological particles) by virtue of their tags, each tag is linked to a plurality of different molecules, generally polypeptides. As a result, upon sorting, the capture agent and binding partner-tagged reagent form a complex and the resulting complex can bind to further molecules. Since the tagged reagents specific for each capture agent can contain a plurality of different molecules that share the same tag, when bound to a plurality of different capture agents the resulting collection presents a highly diverse collection of binding sites. The collection is addressable because the identity of the tags is known or can be ascertained.

As used herein, used to “bind” to a capture system means to interact with sufficient affinity to immobilize the bound moiety (biological particle) temporarily under the conditions of a particular experiment. For purposes herein, it is an interaction that permits molecules and/or biological particles, such as cells, to be retained at a locus when they are contacted with the capture systems so that they no longer move by Brownian motion or other microcurrents in a composition.

A self-assembling array is an addressable collection of capture agents, where the capture agents specifically bind to predetermined binding partners.

As used herein, a self-assembled array is an array that results when a self-assembling array is combined with molecules or biological particles that are conjugated to binding partners specific for the capture agents in a self-assembling array.

As used herein, the components of a self-assembled array include a self assembling array, and binding partners specific therefor or nucleic acids encoding the binding partners or sequence information for synthesis of the binding partners or nucleic acids encoded thereby, and optionally conjugation reagents. As used herein, a capture system refers to an addressable collection of capture agents and polypeptide binding partner-tagged molecules bound thereto, where each different binding partner specifically binds to a different capture agent.

As used herein, antibody refers to an immunoglobulin, whether natural or partially or wholly synthetically, such as recombinantly, produced, including any derivative thereof that retains the specific binding ability of the antibody. Hence antibody includes any protein having a binding domain that is homologous or substantially homologous to an immunoglobulin binding domain. For purposes herein, antibody includes antibody fragments, such as Fab fragments, which are composed of a light chain and the variable region of a heavy chain. Antibodies include members of any immunoglobulin class, including IgG, IgM, IgA, IgD and IgE.

As used herein, a monoclonal antibody refers to an antibody secreted by a hybridoma clone. Because each such clone is derived from a single B cell, all of the antibody molecules are identical. Monoclonal antibodies can be prepared using standard methods known to those with skill in the art (see, e.g., Köhler et al. Nature 256:495 (1975) and Köhler et al. Eur. J. Immunol. 6:511 (1976)). For example, an animal is immunized by standard methods to produce antibody-secreting somatic cells. These cells are then removed from the immunized animal for fusion to myeloma cells.

As used herein, antibody fragment refers to any derivative of an antibody that is less than full length, retaining at least a portion of the full-length antibody's specific binding ability. Examples of antibody fragments include, but are not limited to, Fab, Fab′, F(ab)₂, single-chain Fvs (scFv), Fv, dsFv, diabody and Fd fragments. The fragment can include multiple chains linked together, such as by disulfide bridges.

As used herein, an Fv antibody fragment is composed of one variable heavy domain (V_{H}) and one variable light (V_{L}) domain linked by noncovalent interactions.

As used herein, a dsFv refers to an Fv with an engineered intermolecular disulfide bond, which stabilizes the V_{H}-V_{L} pair.

As used herein, an F(ab)₂ fragment is an antibody fragment that results from digestion of an immunoglobulin with pepsin at pH 4.0-4.5; it can be recombinantly produced.

As used herein, an Fab fragment is an antibody fragment that results from digestion of an immunoglobulin with papain; it can be recombinantly produced.

As used herein, scFvs refers to antibody fragments that contain a variable light chain (V_{L}) and variable heavy chain (V_{H}) covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

As used herein, hsFv refers to antibody fragments in which the constant domains normally present in an Fab fragment have been substituted with a heterodimeric coiled-coil domain (see, e.g., Arndt et al. (2001) J Mol Biol. 7:312:221-228).

As used herein, diabodies are dimeric scFv; diabodies typically have shorter peptide linkers than scFvs, and they preferentially dimerize.

As used herein bispecific antibodies are antibodies constructed to have two antigen binding sites, each for a different antigen or each composed of a different antigen binding site. Bispecific antibodies can be made by fusing hybridoma lines expressing two different antibodies or they can be made through in vitro and recombinant methods to conjugate two antibody fragments containing different antigen binding sites.

As used herein, humanized antibodies refer to antibodies that are modified to include “human” sequences of amino acids so that administration to a human does not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions.

As used herein, a B cell refers to a lymphocyte that develops from hemopoietic stem cells in the bone marrow of adults and the liver of fetuses and is responsible for the production of circulating antibodies.

As used herein, a protein scaffold or polypeptide scaffold refers to any polypeptide or portion thereof that is sufficient to form a conformationally stable structural support, or framework, which is able to display one or more sequences of amino acids that bind an antigen (e.g. CDRs, a variable region) in a localized surface region. A scaffold can be a naturally occurring polypeptide or polypeptide “fold” (a structural motif), or can have one or more modifications, such as additions, deletions or substitutions of amino acids, relative to a naturally-occurring polypeptide or fold. A scaffold can be derived from a polypeptide of any species (or of more than one species), such as a human, other mammal, other vertebrate, invertebrate, plant, bacteria or virus.

As used herein, the term “antibody scaffold” refers to a scaffold of an antibody or of an antibody fragment that contains all or part of an immunoglobulin. Exemplary antibody scaffolds include whole antibodies, and fragments thereof, such as Fv fragments (which can or can not contain an introduced disulfide bond), Fab fragments, Fab′ fragments, F(ab′)2 fragments, and single-chain scFv fragments.

As used herein, phage display refers to the expression of proteins or peptides on the surface of filamentous bacteriophage.

As used herein, panning refers to an affinity-based selection procedure for the isolation of phage displaying a molecule with a specificity for a desired capture molecule or epitope.

As used herein, normalization refers to the equilibration of the titer or concentration of all members of a library, such as a tagged library, so that the number of particular members, such tagged members or total members, in two samples or portions are about the same.

As used herein, staining refers to the visualization of molecules bound to the capture system. Staining can be non-specific, semi-specific or specific depending on what is labelled in a sample and when it is detected. Non-specific staining refers to the labelling of non-fractionated or all components in a particular sample generally, although not necessarily, prior to exposure to the capture system. Semi-specific staining as used herein refers to labelling of a portion of a sample, such as, but not limited to, the proteins located on the cell surface or on cellular membranes, either before, during or after exposure to the capture system. Specific staining as used herein refers to the labelling of a specific component of a sample, typically after the exposure of the sample to the capture system. The stain can be any molecule that associates with and that permits visualization or detection of bound molecules.

As used herein, conjugation refers to the formation of a linkage between two molecules such as between an HAHS polypeptide and another molecule.

The linkage can be any binding interaction, including ionic, or covalent bonding such as by preparing fusion proteins or by chemically conjugating HAHS polypeptide and molecule. Conjugation is effected through an interaction with sufficient affinity (Kₐ typically of at least about 10⁶ I/mol, 10⁷ I/mol, 10⁸ I/mol, 10⁹ I/mol, 10¹⁰ I/mol or greater (generally 10⁸ or greater) such that interaction is stable upon binding of a capture agent to the HAHS polypeptide. Further, the conjugates are such that HAHS polypeptide conjugated to another molecule retains the specificity for the interaction between the HAHS polypeptide and capture agent.

As used herein, cross-linking refers to a method of chemical conjugation for linking molecules. Cross-linking reagents include, but are not limited to, heterobifunctional, homobifunctional and trifunctional reagents, and can be used to introduce, produce or utilize reactive groups, such as thiols, amines, hydroxyls and carboxyls, on one or both of the molecules, which can then be contacted with the other, containing a second reactive group, such as a thiol, amine, hydroxyl and carboxyl, to form a chemical linkage between the two molecules. These reagents can be used to directly or indirectly, such as through a linker, conjugate two or more molecules. Cross-linking can be used, for example, to stabilize binding interactions between two molecules such as between an HAHS polypeptide and another molecule or between an HAHS polypeptide and a capture agent.

As used herein, a fusion protein refers to a polypeptide that contains at least two components, such as a polypeptide of interest and a polypeptide binding partner. Fusion proteins can be produced by expression of nucleic acid in a host cell or in vitro. Fusion proteins also can be produced synthetically.

As used herein, diversity (Div) refers to the number of unique (non-duplicated) molecules in a library, such as a nucleic acid library. Diversity is distinct from the total number of molecules in any library, which is equal to or greater than the diversity.

As used herein, an “even distribution of tags” means that the diversity of molecules to be tagged is approximately equivalent for each of the tags so that in any collection of tagged molecules on average each tagged molecule is unique. As a result, the diversity of different tagged molecules on the loci (spots in a solid phase array) in each array provided herein is approximately the same (i.e., to within, one order of magnitude, or 0.5 orders of magnitude, or 0.25 orders of magnitude or less). In addition, the diversity of different tags at each locus approaches 1, and is typically less than 100, 50, 10 or 5. The tolerance for variation in diversity in tags at each locus is a function of the application of the resulting capture systems or arrays.

As used herein, tagged library refers to the resulting collections of molecules where each of the molecules in the library are separately tagged.

As used herein, a canvas is a collection of arrays, such as those provided herein. The size of each array and number in a canvas can vary and is at least two and is up to a predetermined number, such as q, which is 2 to 10, 20, 30, 50, 100, 200, 250, 300, 500, 1000, 2000, 3000, 4000, 5000, 10,000 and more, including 96 and multiples thereof (i.e., 384, 1 536 and higher densities).

As used herein, a support (also referred to as a matrix support, a matrix, an insoluble support or solid support) refers to any solid or semisolid or insoluble support to which a molecule of interest, typically a biological molecule, organic molecule or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications. The matrix herein can be particulate or can be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads”, are often, but not necessarily, spherical. Such reference, however, does not constrain the geometry of the matrix, which can be any shape, including random shapes, needles, fibers, and elongated. Roughly spherical “beads”, particularly microspheres that can be used in the liquid phase, also are contemplated. The “beads” can include additional components, such as magnetic or paramagnetic particles (see, e.g., Dynabeads® (Dynal, Oslo, Norway)) for separation using magnets, as long as the additional components do not interfere with the methods and analyses herein.

As used herein, matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and can be on the order of cubic microns. Such particles are collectively called “beads.”

As used herein, printing refers to immobilization of capture agents onto a solid support, such as, but not limited to, a microarray.

As used herein, profiling refers to detection and/or identification of a plurality of components, generally 3 or more, such as 4, 5, 6, 7, 8, 10, 50, 100, 500, 1000, 10⁴, 10⁴, 10⁶, 10 ⁷ or more, in a sample. A profile refers to the identified loci to which components of a sample detectably bind. The profile can be detected as a pattern on a solid surface, such as in embodiments when the addressable collection includes an array of capture agents on a solid support, in which case the profile can be presented as a visual image. In embodiments, such as those in which the capture agents and bound tagged molecules are on color-coded beads or are otherwise detectably labeled, a profile refers to the identified polypeptide binding partner tags and/or capture agents to which component(s) is(are) detectably bound, which can be in the form of a list or database or other such compendium.

As used herein, a label is a detectable marker that can be attached or linked directly or indirectly to a molecule or associated therewith. The detection method can be any method known in the art.

As used herein, a fluorescent protein refers to a protein that possesses the ability to fluoresce (i.e., to absorb energy at one wavelength and emit it at another wavelength). These proteins can be used as a fluorescent label or marker and in any applications in which such labels are used, such as immunoassays, CRET, FRET, and FET assays. For example, a green fluorescent protein (GFP) refers to a polypeptide that has a peak in the emission spectrum at about 510 nm. Green, blue and red fluorescent proteins are well known and readily available (Stratagene, see, U.S. Pat. Nos. 6,247,995 and 6,232,107).

As used herein, a subcellular compartment or an organelle is a membrane-enclosed compartment in a eukaryotic cell that has a distinct structure, macromolecular composition, and function. Organelles include, but are not limited to, the nucleus, mitochondrion, chloroplast, and Golgi apparatus.

As used herein, screening refers to the process of analyzing molecules, such as sets of molecules and library compounds, by methods that include, but are not limited to, ultraviolet-visible (UV-VIS) spectroscopy, infra-Red (IR) spectroscopy, fluorescence spectroscopy, fluorescence resonance energy transfer (FRET), NMR spectroscopy, circular dichroism (CD), mass spectrometry, other analytical methods, high throughput screening, combinatorial screening, enzymatic assays, antibody assays and other biological and/or chemical screening methods or any combination thereof.

As used herein, in silico refers to research and experiments performed using a computer. In silico methods include, but are not limited to, molecular modelling studies, biomolecular docking experiments, and virtual representations of molecular structures and/or processes, such as molecular interactions.

As used herein, biological sample refers to any sample obtained from a living or viral source and includes any cell type or tissue of a subject from which nucleic acid or protein or other macromolecule can be obtained. Biological samples include, but are not limited to, body fluids, such as blood, plasma, serum, cerebrospinal fluid, synovial fluid, urine and sweat, tissue and organ samples from animals and plants. Also included are soil and water samples and other environmental samples, viruses, bacteria, fungi, algae, protozoa and components thereof. Hence bacterial and viral and other contamination of food products and environments can be assessed. The methods herein are practiced using biological samples and in some embodiments, such as for profiling, also can be used for testing any sample.

As used herein, combinatorial chemistry is a synthetic strategy that produces diverse, usually large, chemical libraries. It is the systematic and repetitive, covalent connection of a set, the basis set, of different monomeric building blocks of varying structure to each other to produce an array of diverse molecules [see, e.q., Gallop et al. (1994) J. Medicinal Chemistry 37:1233-1251]. It also encompasses other chemical modifications, such as cyclizations, eliminations, cleavages, and other such reactions, that are carried in manner that generates permutations and thereby collections of diverse molecules.

As used herein, macromolecule refers to any molecule having a molecular weight from the hundreds up to the millions. Macromolecules include peptides, proteins, nucleotides, nucleic acids, and other such molecules that are generally synthesized by biological organisms, but can be prepared synthetically or using recombinant molecular biology methods.

As used herein, the term “biopolymer” is a biological molecule, including macromolecules, composed of two or more monomeric subunits, or derivatives thereof, which are linked by a bond or a macromolecule. A biopolymer can be, for example, a polynucleotide, a polypeptide, a carbohydrate, or a lipid, or derivatives or combinations thereof, for example, a nucleic acid molecule containing a peptide nucleic acid portion or a glycoprotein, respectively. Biopolymers include, but are not limited to, nucleic acids, proteins, polysaccharides, lipids and other macromolecules. Nucleic acids include DNA, RNA, and fragments thereof. Nucleic acids can be derived from genomic DNA, RNA, mitochondrial nucleic acid, chloroplast nucleic acid and other organelles with separate genetic material.

A monomeric unit refers to one of the constituents from which a resulting biopolymer or other polymer is built. Thus, monomeric units include, but are not limited to, nucleotides, amino acids, and pharmacophores from which small organic molecules are synthesized.

As used herein, a molecule refers to any compound, including any found in nature and derivatives thereof, including but not limited to, for example, biopolymers, biomolecules, macromolecules and components and precursors thereof, such as peptides, proteins, organic compounds, oligonucleotides or monomeric units of the peptides, organics, nucleic acids and other macromolecules.

As used herein, a biomolecule is any compound found in nature, or derivatives thereof. Biomolecules include, but are not limited to: oligonucleotides, oligonucleosides, proteins, peptides, amino acids, peptide nucleic acids (PNAs), oligosaccharides and monosaccharides.

As used herein, a biological particle refers to a virus, such as a viral vector or viral capsid with or without packaged nucleic acid, phage, including a phage vector or phage capsid, with or without encapsulated nucleic acid, a single cell, including eukaryotic and prokaryotic cells or fragments thereof, a liposome or micellar agent or other packaging particle, and other such biological materials.

As used herein, a secondary agent is a molecule which influences the activity of another molecule either directly or indirectly. Effects of secondary molecules can be in vitro or in vivo. Secondary agent effects include, but are not limited to, stimulation, co-stimulation, inhibition, co-inhibition and competitive effects. Secondary agents include, but are not limited to, an organic compound, inorganic compound, metal complex, receptor, enzyme, protein complex, antibody, protein, nucleic acid, peptide nucleic acid, DNA, RNA, polynucleotide, oligonucleotide, oligosaccharide, lipid, lipoprotein, amino acid, peptide, polypeptide, peptidomimetic, carbohydrate, cofactor, drug, prodrug, lectin, sugar, glycoprotein, biomolecule, macromolecule, an antibody or fragment thereof, antibody conjugate, biopolymer, polymer or any combination, portion, salt, or derivative thereof.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about fifty to one hundred nucleotides in length.

Nucleotide analogs contained in a polynucleotide can be, for example, mass modified nucleotides, which allows for mass differentiation of polynucleotides; nucleotides containing a detectable label such as a fluorescent, radioactive, luminescent or chemiluminescent label, which allows for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well known methods (see, for example, Weiler et al., Nucleic acids Res. 25:2792-2799 (1997)).

As used herein, oligonucleotides refer to polymers that include DNA, RNA, nucleic acid analogues, such as PNA, and combinations thereof. For purposes herein, primers and probes are single-stranded oligonucleotides or are partially single-stranded oligonucleotides.

As used herein, production by recombinant means by using recombinant DNA methods means the use of the well known methods of molecular biology for expressing proteins encoded by cloned DNA.

The term “substantially” identical or homologous or similar varies with the context as understood by those skilled in the relevant art and generally means at least 70%, preferably means at least 80%, more preferably at least 90%, and most preferably at least 95% identity.

As used herein, “reporter” or “reporter moiety” refers to any moiety that allows for the detection of a molecule of interest, such as a protein expressed by a cell, or a biological particle. Typical reporter moieties include, for example, fluorescent proteins, such as red, blue and green fluorescent proteins (see, e.g., U.S. Pat. No. 6,232,107, which provides GFPs from Renilla species and other species), the lacZ gene from E. coli alkaline phosphatase, chloramphenicol acetyl transferase (CAT) and other such well-known genes. For expression in cells, nucleic acid encoding the reporter moiety can be expressed as a fusion protein with a protein of interest or under the control of a promoter of interest.

As used herein, the phrase “operatively linked” generally means the sequences or segments have been covalently joined into one piece of DNA, whether in single- or double-stranded form, whereby control or regulatory sequences on one segment control or permit expression or replication or other such control of other segments. The two segments are not necessarily contiguous. It means a juxtaposition between two or more components so that the components are in a relationship permitting them to function in their intended manner. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression a sequence of nucleotides and a regulatory sequence(s) are connected in such a way as to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, a reporter gene construct is a nucleic acid molecule that includes a nucleic acid encoding a reporter operatively linked to a transcriptional control sequences. Transcription of the reporter gene is controlled by these sequences. The activity of at least one or more of these control sequences is directly or indirectly regulated by a cell surface protein or other protein that interacts with tagged molecules or other molecules in the capture system. The transcriptional control sequences include the promoter and other regulatory regions, such as enhancer sequences, that modulate the activity of the promoter, or control sequences that modulate the activity or efficiency of the RNA polymerase that recognizes the promoter, or control sequences are recognized by effector molecules, including those that are specifically induced by interaction of an extracellular signal with a cell surface protein. For example, modulation of the activity of the promoter may be effected by altering the RNA polymerase binding to the promoter region, or, alternatively, by interfering with initiation of transcription or elongation of the mRNA. Such sequences are herein collectively referred to as transcriptional control elements or sequences. In addition, the construct may include sequences of nucleotides that alter translation of the resulting MRNA, thereby altering the amount of reporter gene product.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, the term “regulatory region” means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present, or at increased concentration, gene expression increases. Regulatory regions also include sequences that confer repression of gene expression (i.e., a substance or stimulus decreases transcription). When a repressor is present or at increased concentration, gene expression decreases. Regulatory regions are known to influence, modulate or control many in vivo biological activities including cell proliferation, cell growth and death, cell differentiation and immune-modulation. Regulatory regions typically bind one or more trans-acting proteins which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of MRNA and, stop codons, leader sequences and fusion partner sequences, internal ribosome entry sites (IRES) for the creation of multigene, or polycistronic, messages, polyadenylation signals to provide proper polyadenylation of the transcript of a gene of interest and stop codons and can be optionally included in an expression vector.

As used herein, regulatory molecule refers to a polymer of deoxyribonucleic acid (DNA) or ribonucleic acid (RNA), or an oligonucleotide mimetic, or a polypeptide or other molecule that is capable of enhancing or inhibiting expression of a gene.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids” which refer generally to circular double stranded DNA loops which, in their vector form are not bound to the chromosome. “Plasmid” and “vector” are used interchangeably as the plasmid is the most commonly used form of vector. Other such other forms of expression vectors that serve equivalent functions and that become known in the art subsequently hereto.

As used herein, a composition refers to any mixture. It can be a solution, a suspension, liquid, powder, a paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof.

As used herein, kit refers to a packaged combination, optionally including instructions and/or reagents for their use.

As used herein, a database refers to a collection of data items.

As used herein, a relational database is a collection of data items organized as a set of formally-described tables from which data can be accessed or reassembled in many different ways without having to reorganize the database tables. Such databases are readily available commercially, for example, from Oracle, IBM, Microsoft, Sybase, Computer Associates, SAP, or multiple other vendors. Databases can be stored on computer-readable media, such as floppy disks, compact disks, digital video disks, computer hard drives and other such media.

### B. GENERATION OF POLYPEPTIDE COLLECTIONS

Provided herein are collections of polypeptides and methods for generating collections thereof. The methods for generating collections of polypeptides include selecting subsets of polypeptides from the total number of possible polypeptides. The subsets can be limited by scale and/or by biasing the collection towards one or more selected properties. Subsets can be limited, for example, by selecting a polypeptide length, thereby limiting the total number of polypeptide members in the subset. Subsets also can be limited by the number of members chosen for the subset, such as by imposing a set of criteria, for example, by choosing a subset of polypeptides which are more similar or dissimilar to each other, by constraining the number of amino acids selected to construct polypeptides of the subset, or by constraining particular positions of polypeptides in the subset. Subsets also can be limited by imposing criteria for a selected property. For example, such criteria can be selected such that the polypeptides have a higher probability of being antigenic in a particular host, and/or have reduced antigenicity in a second host. Selection criteria also can include criteria based on the ease of and success rate of synthesis or high yield of polypeptides, stability, solubility and any other properties desired.

### 1. HANS Polypeptides and Collections Thereof

The methods provided herein can be used to design and generate highly antigenic highly specific (HAHS) polypeptides and collections of HAHS polypeptides. HAHS polypeptides and collections of HAHS polypeptides can be generated by selecting subsets of polypeptides with criteria that result in a higher success rate of antigenicity, such that the members of the collection induce, upon administration to a host, antibodies that are specific for the HAHS polypeptides or upon screening, select for capture agents, such as antibodies, with specific and selective binding to the HAHS polypeptides. Collections of HAHS polypeptides can be generated by imposing criteria which limit the scale of the subset chosen for the collection, such as, but not limited to, selecting a length of the polypeptides, selecting criteria for similarity or dissimilarity of the subset, and selecting number and/or types of amino acids used to construct the subset.

For example, one or more collections of HAHS polypeptides can be generated by:

(a) selecting polypeptides of a length “q”, where q represents the number of amino acids (also referred to as positions) within each polypeptide;

(b) selecting the number of residues within length q which are constrained by selection(s) of amino acids to be represented at each selected position;

(c) selecting the arrangement of positions within each polypeptide;

(d) selecting a subset of polypeptides with the imposed criteria by one or more of steps (a)-(c);

(e) selecting a further subset of polypeptides from step (d) based on a dissimilarity factor.

In one example, HAHS polypeptides and collections thereof have the general formula:
q=m+r
Where q is the total number of amino acids in the polypeptide, and m and r are numbers of amino acids constrained by selected criteria, where the criteria for selection of m and r are independent of each other. Of the selected amino acids, m is the number of critical amino acids and r is the number of non-critical amino acids. Such subsets can be further limited by using a selected number of amino acids which are more likely to be found in antigenic polypeptides. Additional criteria such as the dissimilarity of the members of the set, amino acids selected at particular positions of the polypeptides can be used to further limit the size of the collections as well as bias the collection towards selected properties. q is the total number of amino acids in the polypeptide, m is the number of critical amino acids and r is the number of non-critical amino acids; m is ≧r, with the proviso that it is at least 2. q is at least 4 and can be any length, generally between 4 and 20, 4 and 30, 4 and 50 and 4 and 100. Typically q is at least 5, 6, 7, 8, 9, 10, 15, 20.

For example, in one such method for generating highly antigenic, highly specific binding polypeptides includes:

1) ranking amino acids based upon their frequency in a pre-selected set of antigenic polypeptides, wherein n amino acids are ranked.

2) Based upon the ranking, using x number of amino acids where x is the top n-1 to m amino acids, to produce a set of polypeptides of length m residues; the set containing all combinations of the amino acids in a polypeptide of pre-selected length m residues.

3) Based upon pre-determined criteria for dissimilarity, selecting a subset of set of dissimilar polypeptides.

4) The number of non-critical amino acids, r is chosen to be either zero or an integer of 1 or greater and a number y of amino acids are possible at each of these non-critical positions. Polypeptide sequences of length q are then constructed from the subset of dissimilar polypeptides with m residues each and the addition of r non-critical residues.

Methods also are provided herein for generating or selecting capture agents which bind to highly antigenic highly specific polypeptides. Such methods include introducing collections of HAHS polypeptides into an animal and isolating antibodies as a result of raising an immune response to the introduced HAHS polypeptides. The methods also include selecting capture agents from a collection of candidates for the capture agents which selectively and specifically bind to one or more HAHS polypeptides.

### 2. Description of the Methods

Provided herein are methods for obtaining highly antigenic highly specific (HAHS) polypeptides for use as partners with capture agents such as antibodies.

The polypeptides contain any number of amino acids against which a specific capture agent can be generated, selected or synthesized to bind. Typically such polypeptides are at least 2, 3, 4, 5, 6, 7, 8 to about 100 amino acids in length, usually between 2-50, 2-40, 2-30, 2-20, 4-20, 5-20, 2-50, 4-50, 5-50, and 6-20 amino acids in length. Also provided are methods for generating capture agents, such as antibodies, which bind to HAHS polypeptides. Thus, methods generate pairs of HAHS polypeptides and capture agents. There is no detectable cross-reactivity, such as by ELISA assay, between or among different pairs of HAHS polypeptides and capture agents.

The method of designing HAHS polypeptides constructs or designs polypeptides that contain sequences of amino acids that are antigenic (i.e., they can be more likely to be antigenic than a randomly selected or generated polypeptide of the same or similar size). These polypeptides can be more likely to raise an immune response in a subject and/or bind antibodies or a portion thereof with a high affinity and specificity than a randomly selected polypeptide.

a. Selecting Amino Acids

The methods provided herein and described in detail below, use statistical probabilities that a particular amino acid appears in an antigenic polypeptide.

These statistical probabilities can be calculated or generated empirically. Statistical probabilities for naturally occurring amino acids are exemplified herein. The same or similar methods can be applied to any sets of amino acids including non-naturally occurring amino acids and analogs thereof.

i. Ranking Antigenicity

Ranking of amino acids for antigenicity can be derived empirically or statistically. For example, sequences of antigenic polypeptides can be obtained by empirical methods, such as by injecting mice with polypeptides representing all the possibilities of a set length of polypeptides. The polypeptides are injected into mice and antisera is collected. The antisera then is tested on collections of polypeptides and the antigenic polypeptides are identified based on their reactivity with the antisera. Non-antigenic polypeptides are identified by their lack of reactivity with the antisera. The frequency of an amino acid appearing in a polypeptide that is antigenic is used to determine which amino acids are more likely to be found in an antigenic polypeptide.

The number of polypeptides possible for all sequence combinations is high. For example, a 4 mer has 20×20×20×20 possibilities (160,000 total). It is time consuming, costly and undesirable to test each and every polypeptide to determine its antigenicity. The methods described herein obviate the need for such tedious testings. The methods use a statistical prediction based on the frequency of an amino acid appearing in a polypeptide that is antigenic. The likelihood that an amino acid appears in a polypeptide that is antigenic can be determined based on a representative set of data, for example, based on immunizing animals with a representative subset of all the possibilities of that polypeptide length. Based on the subset of polypeptides injected which are antigenic and non-antigenic, amino acids are identified that either are more likely to be present in antigenic polypeptides or are more likely to be present on non-antigenic polypeptides. The likelihood of a amino acid's presence in an antigenic polypeptide gives an observed antigenic ranking. Using polypeptides of the 20 naturally occurring amino acids, a ranking of antigenicity for each amino acid can be obtained. Similarly, an antigenic ranking of amino acids also can be obtained by mapping epitopes in known proteins. Antibodies to known proteins are used to determine the sequence of amino acids to which they bind, for example by deletion or replacement mutagenesis or by synthesizing subsets of amino acid sequence found within the protein sequence. Antibodies are tested for reactivity with the mutants or with subsets of peptide sequences from the protein. The shortest sequence of amino acids from the protein which retains binding to the antibody defines a linear epitope (see for example, Tainer et al. (1991) Intern. Rev. Immunol. 7:165-188). Epitope mapping can be performed with a representative number of proteins and antibodies and the statistical occurrence of each of the 20 amino acids found in the epitopes is determined to generate the antigenic ranking of the amino acids (see, e.g., Geysen et a., (1988). J. Molecular Recognition 1:32-41; Getzoff et al., (1988). The Chemistry and Mechanism of Antibody Binding to Protein Antigens. Academic Press. Advances in Immunology. Vol 43:1-98). For example, a propensity factor can be calculated by comparing the ratio of the observed frequency of a chosen amino acid appearing in an antigenic polypeptide to the frequency which would be expected if it appeared by chance alone (Geysen et al., (1988). J. Molecular Recognition 1:32-41). Epitope mapping and antigenic ranking such as with known proteins or by injecting collections of random polypeptides can be done in any species of interest that raises an immune response, for example mice, rabbit, rat, human, monkey, dog, chicken, and goat. For example, using data obtained from epitope mapping (Geysen et al., (1988). J. Molecular Recognition 1:32-41), the amino acids were assigned the following antigenic rankings, with 1 being the highest and 20 the lowest probability (Table 2).

TABLE 2
Antigenic Ranking

| Ranking | amino acid |
|---|---|
| 1 | E |
| 2 | P |
| 3 | Q |
| 4 | N |
| 5 | F |
| 6 | H |
| 7 | T |
| 8 | K |
| 9 | L |
| 10 | D |
| 11 | V |
| 12 | I |
| 13 | G |
| 14 | Y |
| 15 | S |
| 16 | C |
| 17 | A |
| 18 | M |
| 19 | R |
| 20 | W |

Antigenic ranking can be obtained using data from a single species or multiple species. Antigenic ranking also can compare antigenicity between hosts such that HAHS polypeptides can be generated which are antigenic in one species but less antigenic or non-antigenic in another species. For example, antigenicity rankings can reflect high antigenicity in mice but lower antigenicity in humans.

Epitope mapping and antigenic ranking also can be performed using recombinant means, by screening libraries of antibodies or antibody fragments with polypeptides containing sequences of epitopes, such as collections of sequences of critical amino acids. The polypeptides which are bound by the antibodies can be identified and the frequency of the amino acids appearing in polypeptides bound by the antibodies can be determined. Experimental conditions such as washing conditions in a phage library panning assay can be used to control the affinity of the interaction between the antibodies and the peptides.

ii. Generating Polypeptides with Chosen Amino Acids

A length, “m”, is selected for a set of polypeptides. Polypeptides can be any length sufficient for an antibody epitope, generally less than 20 amino acids. For example, the polypeptides length is between 2 and 20 amino acids, such as 2, 3, 4, 5, 6, 7, 8, 9, 10, 11, 12, 13, 14, 15, 16, 17, 18, 19 and 20 amino acids in length. In one exemplary embodiment, 4-mers are selected.

A threshold ranking of antigenicity can be chosen to limit the possible number of polypeptides in the subset (subset A) and to bias the subset to more antigenic sequences. For example, if the polypeptide length is 20 amino acids, each of the 20 positions can be selected from the top 19 antigenic ranking amino acids, limiting the subset from the total possibilities of all 20 amino acids at each position. The threshold can be set according to the number of polypeptides desired in the subset and the level of dissimilarity chosen for the subset. In one embodiment, the amino acids are chosen from the top n-1 antigenic ranking amino acids, where n is the total amino acids in a ranked set. For example, antigenic amino acids are chosen from the set of 20 naturally-occurring amino acids. In one aspect of the embodiment, the top 19, 18, 17, 16, 15, 14, 13, 12, 11, 10, 9, 8, 7, 6, or 5 antigenic ranking amino acids are used to design and construct the polypeptide sequences. In one exemplary embodiment, the top 10 antigenic ranking amino acids are used to design and construct polypeptide sequences in subset A. In another exemplary embodiment, the amino acids E, P, Q, N, F, H, T, K, L, and D are used to design and construct polypeptide sequences.

iii. Use of Non-Naturally Occurring Amino Acids

Antigenic amino acids can include natural and/or non-natural amino acids, such as non-natural amino acids described further herein. Non-naturally occurring amino acids can be ranked for antigenicity using methods applied to the naturally occurring amino acids, for example by testing sequences against antisera or libraries of antibodies (described herein) and can be ranked along-side naturally occurring amino acids. For example, a representative set of polypeptides composed of non-naturally occurring amino acids and/or a combination of non-naturally occurring and naturally occurring amino acids of a chosen polypeptide length can be used to immunize animals. Based on the subset of polypeptides injected which are antigenic and non-antigenic, amino acids are identified which either are more likely to be present in antigenic polypeptides or are more likely to be present on non-antigenic polypeptides. The likelihood of a amino acid's presence in antigenic polypeptide gives an observed antigenic ranking. Some non-natural amino acids are very structurally similar to naturally occurring amino acids and to other non-naturally occurring amino acids. This similarity can be factored in to provide antigenicity rankings based on these similarities. For example, a collection of polypeptides can be generated containing non-natural amino acids and tested for antigenicity. Polypeptides which are antigenic can be used to create further sets of polypeptides (replacement sets) by systematically replacing some or all of the amino acids systematically to determine which amino acids are critical. The data can then be analyzed for the replacement sets to determine a factor for each non-natural amino acid, where the factor represents the frequency of finding the particular non-natural amino acid in a critical position within an antigenic polypeptide.

The use of non-naturally occurring amino acids increases the diversity and thus uniqueness of the polypeptides that can be generated. For example, there are several hundred non-naturally occurring amino acids that are commercially available and a even larger number that can be synthesized by standard chemistry methods known in the art. Non-naturally occurring amino acids can be used at either critical or non-critical residues or at both critical and non-critical residues. The ability to incorporate non-naturally occurring amino acids also permits linear, cyclic and branched polypeptide structures to be designed and constructed.

Non-natural amino acids include, but are not limited to, non-natural ,β-amino acids; amino acids having alkyl, cycloalkyl, heterocyclyl, aromatic, heteroaromatic, electroactive, conjugated, azido, carbonyl and unsaturated side chain functionalities; isomeric N-substituted glycine, wherein the side chain of an α-amino acid is attached to the amino nitrogen instead of to the α-carbon of that molecule. The following are representative non-limiting examples of non-natural amino acids:

Non-natural amino acids that are modifications of natural amino acids such that the amino group is attached to β-carbon atom of the natural amino acid (e.g. β-tyrosine). Non-natural amino acids that are modifications of natural amino acids in the side chain functionality, such that the imino groups or divalent non-carbon atoms such as oxygen or sulfur of the side chain of the natural amino acids have been substituted by methylene groups, or, alternatively, amino groups, hydroxyl groups or thiol groups have been substituted by methyl groups, olefin, or azido groups, so as to eliminate their ability to form hydrogen bonds, or to enhance their hydrophobic properties (e.g. methionine to norleucine).

Non-natural amino acids that are modifications of natural amino acids in the side chain functionality, such that the methylene groups of the side chain of the natural amino acids have been substituted by imino groups or divalent non-carbon atoms or, alternatively, methyl groups have been substituted by amino groups, hydroxyl groups or thiol groups, so as to add ability to form hydrogen bonds or to reduce their hydrophobic properties (e.g. leucine to 2-aminoethylcysteine, or isolecine to o-methylthreonine).

Non-natural amino acids that are modifications of natural amino acids in the side chain functionality, such that a methylene group or methyl groups have been added to the side chain of the natural amino acids to enhance their hydrophobic properties (e.g. Leucine to gamma-Methylleucine, Valine to beta-Methylvaline (t-Leucine)).

Non-natural amino acids that are modifications of natural amino acids in the side chain functionality, such that a methylene groups or methyl groups of the side chain of the natural amino acids have been removed to reduce their hydrophobic properties (e.g. Isoleucine to Norvaline).

Non-natural amino acids that are modifications of natural amino acids in the side chain functionality, such that the amino groups, hydroxyl groups or thiol groups of the side chain of the natural amino acids have been removed or methylated to eliminate their ability to form hydrogen bonds (e.g. Threonine to o-methylthreonine or Lysine to Norleucine). Non-natural amino acids that are optical isomers of the side chains of natural amino acids (e.g. Isoleucine to Alloisoleucine).

Non-natural amino acids that are modifications of natural amino acids in the side chain functionality, such that the substituent groups have been introduced as side chains to the natural amino acids (e.g. Asparagine to beta-fluoroasparagine). Non-natural amino acids that are modifications of natural amino acids where the atoms of aromatic side chains of the natural amino acids have been replaced to change the hydrophobic properties, electrical charge, fluorescent spectrum or reactivity (e.g. Phenylalanine to Pyridylalanine, Tyrosine to p-Aminophenylalanine).

Non-natural amino acids that are modifications of natural amino acids where the rings of aromatic side chains of the natural amino acids have been expanded or opened so as to change hydrophobic properties, electrical charge, fluorescent spectrum or reactivity (e.g. Phenylalanine to Naphthylalanine, Phenylalanine to Pyrenylalanine). Non-natural amino acids that are modifications of the natural amino acids in which the side chains of the natural amino acids have been oxidized or reduced so as to add or remove double bonds (e.g. Alanine to Dehydroalanine, Isoleucine to Beta-methylenenorvaline).

Non-natural amino acids that are modifications of proline in which the five-membered ring of proline has been opened or, additionally, substituent groups have been introduced (e.g. Proline to N-methylalanine). Non-natural amino acids that are modifications of natural amino acids in the side chain functionality, in which the second substituent group has been introduced at the alpha-position (e.g. Lysine to alpha-difluoromethyllysine).

Non-natural amino acids that are combinations of one or more alterations, as described supra (e.g. Tyrosine to p-Methoxy-m-hydroxyphenylalanine). Non-natural amino acids that are isomeric N-substituted glycines, wherein the side chain of an α-amino acid is attached to the amino nitrogen instead of to the α-carbon of that molecule (e.g. N-methyl glycine, N-isopropyl glycine). Non-natural amino acids which differ in chemical structures from natural amino acids but are compatible, in protected or unprotected form, with a hybrid synthesis of peptide chemistry. Non-natural amino acids are readily available and widely known. Exemplary non-natural amino acids (with their abbreviations) include, but are not limited to, for example: Aib for 2-amino-2-methylpropionic acid, β,-Ala for,β-alanine, α-Aba for L-α-aminobutanoic acid; D-α-Aba for D-α-aminobutanoic acid; Ac₃c for 1-aminocyclopropane-carboxylic acid; Ac₄c for 1-amino-cyclobutanecarboxylic acid; Ac₅c for 1-aminocyclopentanecarboxylic acid; Ac₆c for 1-aminocyclohexanecar-boxylic acid; Ac₇c for 1-aminocycloheptanecarboxylic acid; D-Asp(ONa) for sodium D-aspartate; D-Bta for D-3-(3-benzo[b]thienyl)ala-nine; C₃al for L-3-cyclopropylalanine; C₄al for L-3-cyclobutylalanine; C₅al for L-3-cyclopentylalanine; C₆al for L-3-cyclohexylalanine; D-Chg for D-2-cyclohexylglycine; CmGly for N-(carboxymethyl)glycine; D-Cpg for D-2-cyclopentylglycine; CpGly for N-cyclopentylglycine; Cys(O₃Na) for sodium L-cysteate; D-Cys(O₃H) for D-cysteic acid; D-Cys(O₃Na) for sodium D-cysteate; D-Cys(O₃Bu₄N) for tetrabutylammonium D-cysteate; D-Dpg for D-2-(1,4-cyclo-hexadienyl)-glycine; D-Etg for (2S)-2-ethyl-2-(2-thienyl)glycine; D-Fug for D-2-(2-furyl)glycine; Hyp for 4-hydroxy-L-proline; IeGly for -[2-(4-imidazolyl)ethyl]glycine; alle for L-L-alloisoleucine; D-alle for D-alloisoleucine; D-Itg for D-2-(isothiazolyl)glycine; D-tertLeu for D-2-amino-3,3-dimethylbutanoic acid; Lys(CHO) for N⁶-formyl-L-lysine; MeAla for N-methyl-L-ala-nine; MeLeu for N-methyl-L-leucine; MeMet for N-methyl-L-methionine; Met(O) for L-methionine sulfoxide; Met(O₂) for L-methionine sulfone; D-Nal for D-3-(1-naphthyl)alanine; Nle for L-norleucine; D-Nle for D-nor-leucine; Nva for L-norvaline; D-Nva for D-norvaline; Orn for L-ornithine; Orn(CHO) for N⁵-formyl-L-ornithine; D-Pen for D-penicillamine; D-Phg for D-phenylglycine; Pip for L-pipecolinic acid; ⁱPrGly for N-isopropylglycine; Sar for sarcosine; Tha for L-3-(2-thienyl)alanine; D-Tha for D-3(2-thienyl)-alanine; D-Thg for D-2-(2-thienyl)glycine; Thz for L-thiazolidine-4-carboxy-lic acid; D-Trp(CHO) for Nⁱⁿ-formyl-D-tryptophan; D-trp(O) for D-3-(2,3-di-hydro-2-oxoindol-3-yl)alanine; D-trp((CH₂)ₘCOR¹) for D-tryptophan substituted by a -(CH₂)ₘCOR¹ group at the 1-position of the indole ring; Tza for L-3-(2-thiazolyl)alanine; D-Tza for D-3-(2-thiazolyl)alanine; D-Tzg for D-2-(thiazolyl)glycine.

b. Biased Subsets of Polypeptides

Optionally, in a given length of polypeptide, further bias can be introduced into a set of polypeptides to increase the uniqueness of each polypeptide in the set (subset B). This increase can increase the selectivity and specificity of capture agents which recognize the polypeptides such as by reducing potential cross reactivity between capture agents and polypeptides outside the partner pairs. In one exemplary embodiment, all of the amino acids are different from one another, such that there are no duplicated amino acids within each polypeptide. This further reduces the number of polypeptides in the set (designated as subset B after this bias is imposed). For example, if the polypeptide is a 4-mer and 10 amino acids are chosen from the antigenic ranking list, the possible 4-mers in subset A would be 10×10×10×10. Introducing a bias where each amino acid of the 10 chosen are used only once generates 10×9×8×7 possibilities in subset B, where each amino acid is unique within a 4-mer (i.e., there is no duplication or any multiples of a chosen amino acid within the polypeptide length). Thus, for a 4-mer subset B contains 5040 polypeptides.

c. Critical and Non-Critical Amino Acids

Subset B represents a set of polypeptides of chosen length “m” with amino acids chosen from a set of antigenically ranked amino acids. Optionally, these polypeptides can be incorporated in larger polypeptides, such that the polypeptides derived from subset B are designated the critical residues in polypeptides of subset C. Subset C is composed of “m” critical amino acids and the remaining number of positions “r” in the polypeptide length are noncritical positions. The total length of polypeptides in subset C is “q” residues, where q=m+r. Length “q” of such polypeptides can be generally less than 50 amino acids, typically less than 20 amino acids. For example, the polypeptides length can be between 2 and 20 amino acids, such as 2, 3, 4, 5, 6, 7, 8, 9, 10, 11, 12, 13, 14, 15, 16, 17, 18, 19 and 20 amino acids in length.

A non-critical position does not determine the affinity or specificity of binding to a capture agent for a HAHS polypeptide such that noncritical residues can be replaced by another amino acid without substantially affecting the affinity or specificity of binding of the HAHS polypeptide and capture agent. Generally, non-critical positions can be replaced with a larger set of amino acids. For example, when taken from the set of naturally occurring amino acids, non-critical positions can be replaced usually 10 or more amino acids or in some cases, by any other amino acid from the set of naturally occurring amino acids.

The number of non-critical residues “r” can be zero or any integer greater than or equal to one. In one embodiment, the number of critical residues is larger than the number of non-critical residues. For example, generally for peptides of 9 or less amino acids in length (q), the number of critical residues is approximately 55%, 60%, 70%, 80%, 85%, 90% or 95% of the total number of amino acids in the polypeptide.

The non-critical positions can be designated at specific sites within the polypeptide length to construct subset D. For example, in a polypeptide of total length “q” amino acids, there are “m” critical residues and “r” non-critical residues. Critical residues can all be contiguous, or they can be interspersed with non-critical residues. For example, it can be designated that the N and C terminal residues of the polypeptide are critical residues. In another example, it can be designated that the non-critical residues are found in pairs. In one exemplary embodiment 6-mer polypeptides are designed whereby the first and last (N and C terminal) positions are critical residues and 2 additional positions of the remaining 4 residues of the 6-mer also are critical residues chosen from a set of antigenic amino acids. The remaining 2 positions are non-critical residues and are designated to be in adjacent positions in the 6-mer.

In the above example, the following possibilities are generated for subset D:

$$\begin{matrix}X & N & N & X & X & X \\ X & X & N & N & X & X \\ X & X & X & N & N & X\end{matrix}$$

where X's are critical residues and N's are non-critical residues and the 3 sequences show the possible arrangement to generate polypeptide sequences with adjacent non-critical residues and critical residues at the N and C termini.
d. Selecting a Dissimilar Set

Subset D can then be further restricted to generate a new subset of polypeptides, subset E, that are dissimilar from each other. To extract a subset E, a single polypeptide is chosen at random from subset D as a reference polypeptide. A similarity ranking is calculated for all of the polypeptides in subset D using a replaceability matrix )also referred to herein as a similarity matrix) which compares the similarity of the amino acids at the critical positions to each other (see e.g., Geysen et al. (1988) J. Mol. Recog. 1 (1): 32-41).

A similarity (replaceability) matrix can be constructed empirically. For example, a collection of protein antigens and antisera and/or antibodies which bind to the antigens is generated. The binding sites within the antigens for the antibodies, epitopes, are identified. Such epitopes can be identified by methods such as deletion analysis where amino acids are deleted until the smallest epitope(s) are identified. Epitopes also can be identified by scanning analysis where overlapping sets of polypeptides composed of the possible amino acid oligomers, e.g. 5-mers, 6-mers, 7-mers, or 8-mers etc., of the full-length polypeptide are generated and the antigenic oligomers identify epitopes. One identified, each epitope is then further analyzed by synthesizing the epitope along with a set of peptide analogs which replace each residue with other amino acids. For example, a set can be constructed which replaces each residue, one at a time, with the other 19 naturally occurring amino acids. Such replacement sets also can be constructed with non-naturally occurring amino acids or a combination of naturally occurring and non-naturally occurring amino acids. Such sets can be constructed for example, using combinatorial peptide libraries (Pinilla et al. (1999) Curr. Opin. Immunol. 11:193-202), and multipin synthesis (Geysen et al., (1987) J. Immunol. Methods 102:259-274, Rodda et al. (1996) Methods: A companion to Methods Enz. 9: 473-481). Alternatively, mutagenesis can be used to introduce amino acid changes in the protein containing the epitope, and the effect of the changes assessed to determine replaceability (Alexander et al., (1992) Proc. Natl. Acad. Sci. USA 89:3352-3356). Using the replacement sets, the variants are each tested against antibodies for the epitope and binding is assessed as compared to the unaltered epitope, for example by using an ELISA assay. The comparison of the variants and unaltered epitopes generates scores (for example, scores based on comparison of antigenicity) which can then be integrated with scores from other antigen replacement sets and antibodies to generate a database of replaceability in epitopes and produce a replaceability (similarity) matrix (Geysen et al. (1988) J. Mol. Recog. 1(1): 32-41). Replaceability scores can be based, for example, on the frequency that an amino acid when used to replace another maintains or decreases antigenicity of an epitope.

Non-naturally occurring amino acids also can be assigned a similarity ranking for use with the methods. For example, a similarity matrix can be constructed based on their structural and functional similarity to each other and to naturally occurring amino acids. A similarity matrix also can be constructed by replacing naturally occurring amino acids in epitopes with non-natural amino acids and assessing the binding of antibodies to the replacement epitopes such as by ELISA. An example of a similarity (replaceability) matrix is given in Table 3 (Geysen et al. (1 988) J. Mol. Recog. 1(1): 32-41):

TABLE 3
Similarity Matrix

| | E | P | Q | N | F | H | T | K | L | D | G | S | Y |
|---|---|---|---|---|---|---|---|---|---|---|---|---|---|
| E | 100 | 13 | 33 | 13 | 2 | 8 | 10 | 6 | 8 | 42 | 13 | 15 | 6 |
| P | 5 | 100 | 16 | 11 | 8 | 11 | 11 | 16 | 3 | 3 | 14 | 14 | 0 |
| Q | 15 | 10 | 100 | 25 | 5 | 10 | 10 | 5 | 5 | 5 | 20 | 15 | 10 |
| N | 4 | 0 | 13 | 100 | 4 | 9 | 4 | 9 | 4 | 4 | 4 | 9 | 0 |
| F | 11 | 11 | 11 | 11 | 100 | 5 | 26 | 5 | 37 | 16 | 0 | 32 | 21 |
| H | 8 | 23 | 23 | 15 | 0 | 100 | 15 | 15 | 0 | 0 | 23 | 8 | 8 |
| T | 15 | 6 | 12 | 12 | 6 | 9 | 100 | 12 | 9 | 6 | 3 | 44 | 6 |
| K | 0 | 3 | 26 | 23 | 10 | 26 | 23 | 100 | 10 | 10 | 10 | 29 | 0 |
| L | 2 | 4 | 12 | 6 | 22 | 8 | 4 | 18 | 100 | 8 | 2 | 4 | 10 |
| D | 50 | 4 | 12 | 42 | 4 | 23 | 15 | 0 | 4 | 100 | 0 | 27 | 0 |
| G | 3 | 0 | 9 | 3 | 6 | 12 | 3 | 12 | 6 | 6 | 100 | 24 | 3 |
| S | 17 | 6 | 0 | 0 | 11 | 39 | 22 | 11 | 6 | 0 | 6 | 100 | 6 |
| Y | 0 | 0 | 0 | 0 | 29 | 0 | 0 | 14 | 14 | 0 | 0 | 0 | 100 |

A similarity score is determined for each polypeptide in subset D as compared with the reference polypeptide chosen for subset E. The similarity score can be determined for example, by combining the similarity probabilities from the chosen or generated similarity matrix (represented in Table 3 above as 0-100%) to determine an overall score for the polypeptide. For example, if subset D is a collection of 6-mer polypeptides and a reference polypeptide chosen is as EPNGYF (SEQ ID NO:1), each polypeptide in subset D is compared with this reference polypeptide, EPNGYF (SEQ ID NO:1), using the similarity matrix to calculate a similarity score by combining the similarity value at each of the critical positions to the corresponding positions in the reference polypeptide. The maximum score is 100% (identical polypeptide) and the minimum score is zero.

The number of members for subset E is set at a desired number of polypeptides, for example 10, 20, 30, 40, 50, 100, 200 or 1000 polypeptides. A threshold value is determined which will generate the desired number of polypeptides for subset E. For example, if the threshold is set very low, and therefore the degree of similarity is very low, a smaller subset E of polypeptides will be generated. Conversely, if the threshold of similarity is set high, the subset E will be a larger number of polypeptides. The number of polypeptides can be determined by one skilled in the art based on the intended subsequent use of the polypeptides. For example, if a library of polypeptides of several thousand polypeptides is desired, the threshold can be set higher. If only 10 polypeptides are desired which are dissimilar from each other, the threshold can be set lower.

e. Limiting the Amino Acids Chosen for Non-Critical Positions

From subset E, amino acids are added into the non-critical positions to create subset F. Non-critical positions can be any amino acid, including naturally occurring and non-natural amino acids. Non-critical positions also can be utilized to introduce added functionalities into the polypeptide, such as enhancing solubility and folding. In one exemplary embodiment, amino acids which increase solubility and permit flexibility and folding are used at the non-critical positions. For example, the amino acids S, G and Y are utilized at the non-critical positions. The non-critical positions can be further restricted by designating each as unique, i.e., there is no duplication or any multiples of a chosen amino acid within the polypeptide length. For example, in a given set, such as the exemplary subset of 6-mers described herein, the two non-critical positions are designated as S and G. Non-critical positions also can include additional amino acids at either the N or C terminus. For example, one or more amino acids can be added at either or both termini.

### 3. Production of HAHS Polypeptides

Once subsets of polypeptides are designed, any of the subsets of polypeptides described herein can be generated by standard methods known in the art. The peptides can be chemically synthesized by standard and/or combinatorial chemistry. Polypeptides also can be synthesized using recombinant means such as by expression of nucleic acids encoding the polypeptide sequences. For recombinant expression, polypeptides can be limited to the 20 naturally occurring amino acids and additionally non-naturally occurring amino acids where the expression organism of choice has been genetically engineered to generate such modifications or by in vitro transcription/translation systems (see for example, Budisa et al. ((1998) Proc. Natl. Acad. Sci. 95:455-59; Chin et al. (2003) Science 301:964-967; Klick et al. (2002) PNAS 99:19-24; Kowal et al. (1997) Nuc. Acids Res. 25:4685-4689). Synthesized peptides can be linear, cyclized and branched. For chemically synthesized peptides, selection can be based on compatibility with synthesis techniques, for example based upon particular amino acid stability in a protected or non-protected form to the conditions selected for synthesis. Such conditions are known to one of skill in the art (see for example Geysen et al. (1987) J. Immunol. Methods 102: 259-274; Rodda et al. (1996) Methods: A Companions to Methods in Enz. 9:473-481).

Methods for Preparing Collections of HAHS Polypeptides in a Addressable Format

Provided herein are methods for preparing collections of HAHS polypeptides in an addressable format. The methods are flexible for collection size and include preparation of small and large polypeptide collections, including large diverse collections of HAHS polypeptides, addressably formatted and displayed suitable for screening and other assays.

The methods utilize an addressable format provided by a collection of pairs of tags and capture agents. A collection contains pairs of molecules such that each tag binds a unique capture agent in the collection and each capture agent binds a unique tag in the collection. The total number of tag:capture agent pairs in a collection is designated “b.”

The methods use standard peptide synthesis chemistry known in the art, such as solid phase peptide synthesis methods and are compatible with computer-controlled automated systems. Solid supports contain an insoluble material that is chemically unreactive to the compounds used in synthesis. Examples of such supports include, but are not limited to, polystyrene. The tags are bound to the solid supports such as by a polyethylene glycol linker, which permits cleavage of the products after synthesis. Protecting groups such as, but not limited, to Fmoc, t-butyl and trityl groups are employed to block side chains and functional groups on the amino acids and peptides. Repetitive rounds of protection and chain elongation, along with washing and filtering generate extended peptide chains.

The tags are used as a starting material for peptide synthesis using standard solid-phase peptide chemistry. The C terminus of each tag is linked to a solid support, for example a latex bead. Such linkages can include optionally a first linker between the solid support and the tag and optionally, a second linker between the tag and the peptide synthesis product. The N-terminal group of the tag or second linker is used as the starting point for peptide synthesis.

The tags are gridded out or otherwise addressed for synthesis such that each tag occupies a unique address. For example, a microtiter plate is used as a synthesis block with unique tags conjugated to beads in each well. The tag-bead conjugates can be distributed to the wells or for example, beads can be distributed to all wells and each tag added to a different well and then conjugated. In another example, tags can be physically linked to a solid support and arranged in a grid. Any method known in the art can be used for addressing tag-solid support conjugates so long as the address for each tag is known or can be determined.

Peptides to be synthesized on the tags are of a length d, where d=(number of fixed positions)×(number of variable positions). Fixed positions refers to positions where all of the peptides to be synthesized have the same amino acid at that position; variable positions refers to positions where each peptide to be synthesized will not receive the same amino acid but will receive one of a set of amino acids designated for that position. For example, in synthesizing a collection of HAHS polypeptides, the collection can contain variable positions that correspond to critical amino acids of the HAHS polypeptide and each variable position receives an amino acid designated from a set of antigenically ranked amino acids. The remaining positions can be designated fixed positions corresponding to noncritical amino acids, each receiving a designated amino acid at that position.

The first round of synthesis generates peptides of the formula tag-L-N_{f}-A-N_{f}-B-N_{f}. A and B are two variable positions. Optionally, any number of fixed positions designated N_{f}, can be added before the first variable position A, between variable position A and the second variable position B, where f is zero or an integer between 1 and 10, typically less than 5. The number of fixed positions before A, between A and B and after B are independently chosen. L is an optional linker. A second round of synthesis extends the peptides as represented by the formula tag-L-N_{f}-A-N_{f}-B-N_{f}-C-N_{f}-D-N_{f}. In one exemplary embodiment, peptides of the formula tag-linker-A-N-N-B-C-D are synthesized.

For each variable position, a set of amino acids is chosen, each set represents all of the possible amino acids to be added at that position. Such sets can be chosen from any sets of amino acids, including natural and non-natural amino acids, and subsets of amino acids such as a subset of antigenically ranked amino acids. The number of amino acids chosen for use in synthesizing the A and B positions is set by the total number of available tag:capture agent pairs, b, such that b=X_{A} ×X_{B} where X_{A} is the number of amino acids in the set of amino acids designated for position A and X_{B} is the number of amino acids in the set of amino acids designated for position B. Positions A and B can use the same set of amino acids such that X_{A}=X_{B}. Positions A and B can use a different set of amino acids from each other, such that X_{A} and X_{B} are the same or different.

The addressed tagged beads can be arranged such that a grid is formed of X_{A} columns×X_{B} rows, for example using a microtiter plate. X_{A} amino acids are distributed such that each column receives a unique amino acid from the collection of X_{A} amino acids for synthesis at position A. Following synthesis at position A, a number of fixed positions N_{f} are synthesized where f is zero or an integer between 1 and 10, typically less than 5. A second variable position B is synthesized such that X_{B} rows each receive a unique amino acid from the collection of X_{B} amino acids. Each unique tag now has a unique polypeptide containing a unique combination of amino acids at A and B. For example, if 10 amino acids were chosen for position A and 8 amino acids at position B, the synthesis would produce 80 A-B combinations each of the 80 possibilities linked to a unique tag.

Although for ease of description, the synthesis format is described as a grid, other synthesis formats can be used, so long as each tag receives a unique A-B combination. For example, any format that permits distribution of each amino acid in the set designated for position A to a number of tags equivalent to X_{B} and then distributes each amino acid in the set designated for position B to a number of tags equivalent to X_{A}, such that each tag receives a unique A-B combination and the A-B combination linked to the tag is known or can be determined, is suitable.

Following synthesis at position B, a number of fixed positions N_{f} are synthesized where f is zero or an integer between 1 and 10, typically less than 5. A second round of synthesis is initiated by mixing all the tagged polypeptides of the first round together and distributing them to a grid or otherwise divided synthesis container of b positions.

The second round of synthesis adds an additional two variable positions, C and D. The number of amino acids chosen for the positions is set by the total number of available tag:capture agent pairs, b, such that b=X_{C}×X_{D} where X_{C} is the number of amino acids in the set of amino acids designated for position C and X_{D} is the number of amino acids in the set of amino acids designated for position D. Positions C and D can use the same set of amino acids such that X_{C} =X_{D}. Positions C and D can use a different set of amino acids from each other, such that X_{C} and X_{D} are the same or different. C and D can have the same or different sets of amino acids as used for positions A and B.

The tagged beads with positions A and B and optionally, a number of fixed position, are distributed for the second round of synthesis, for example as a grid of X_{C} columns×X_{D} rows, such that each combination of AB is represented at each position in the grid. The third variable C is synthesized such that X_{C} amino acids are distributed so that each column receives a unique amino acid from the collection of X_{C} amino acids for synthesis at position C. Following synthesis at position C, a number of fixed positions N_{f} are synthesized where f is zero or an integer between 1 and 10, typically less than 5. A fourth variable position D is synthesized such that X_{D} rows each receive a unique amino acid from the collection of X_{D} amino acids.

The second round of synthesis results in tagged AB peptides further extended with a unique combination at positions C and D, such that each AB combination has been extended with each CD possibility. A total of b×b polypeptides possibilities have been synthesized. The AB positions are identifiable by the tags, since each AB possibility is linked to a unique tag. The CD positions are identifiable by their position in the second round synthesis, each address represents a unique CD combination.

At the completion of synthesis, tagged peptides can be cleaved from the solid support. The tagged peptides are sorted by incubating them with the corresponding b number of capture agents, each binding a unique tag. Capture agents can be addressable by positional array or by virtue of a second tag such as an electronic, chemical, optically or color-coded bead, attached to each capture agent.

Peptides synthesized at each address in the second round synthesis are incubated with separate collection of addressed capture agents, such that there are b collections of addressed capture agents, each containing the same capture agents. For example, a canvas of b capture agent arrays is used where peptides from each address at the second round are incubated with a separate array on the canvas. Such distributions generate collections of capture agents, each collection displaying a subset of the synthesized peptides and together displaying the full set of synthesized peptides. Each collection of capture agents displays peptides with a unique CD combination and the full assortment of possibilities at the A and B positions.

The displayed collections of synthesized peptides can be used for screening, for example, to screen against a collection or library of antibodies, antibody fragments, synthetic antibodies or antisera for antibodies and antibody fragments which specifically bind a displayed peptide. In one example, HAHS polypeptides can be synthesized and displayed and a library of single chain antibodies can be screened to identify HAHS peptides and antibodies which bind them. Such method enable screening of synthesized peptides to assess specific binding to other molecules, and in addition to assess cross-reactivity of the displayed peptides. Additionally, the collections of synthesized peptides can be used to screen specific-binding and cross-reactivity of an antibody, antisera, collections and libraries of antibodies, antibody fragments, antisera and other collections of binding proteins.

In an example of the above method, a 6-mer polypeptide is synthesized with 4 variable (A,B,C,D) and two fixed positions (N). Polypeptides of the formula ANNBCD are synthesized with 10 amino acids chosen for each variable position. The 10 amino acids are chosen from a set of antigenically ranked amino acids to be the top 10 ranked amino acids, respectively, and the same 10 amino acids are used in the synthesis of positions A, B, C and D. The first N position is chosen as a glycine and the second N position is chosen as a serine.

Pairs of tags and capture agent pairs are assembled and conjugated to beads. The number of pairs is chosen to be 100 (b=100). The 100 tags are conjugated to a solid support, such as latex beads, and distributed to the wells of a plate, gridded so that they are arranged in a predetermined 10×10 or other suitable format, predetermined so that it is known which tag is at which position in the grid. The tags can optionally be conjugated to a linker such as GS or GSG., such that the synthesized peptide is represented tag-GS-ANNBCD or tag-GSG-ANNBCD, respectively. Standard solid-phase peptide synthesis chemistry is employed to synthesize the tagged peptides.

Position A is synthesized by adding 10 amino acids to the synthesis grid as follows. Each row receives 1 amino acid, such that all positions in the row receive the same amino acid and each different row receives a different amino acid. (e.g. row 1 receives amino acid 1, row 2 receives amino acid 2 etc). The N positions are then synthesized where each row and column position receives the same amino acid for each of the N positions for example a glycine for the first N position, followed by a serine for elongation at the second N position. Position B is synthesized by adding the 10 amino acids designated for position B to the grid where each column receives 1 amino acid, such that all positions in the column receive the same amino acid and each different column receives a different amino acid. (e.g. column 1 receives amino acid 1, column 2 receives amino acid 2 etc). The peptides synthesized, represented by the formula tag-linker-A-GS-B, are removed from the grid and mixed together.

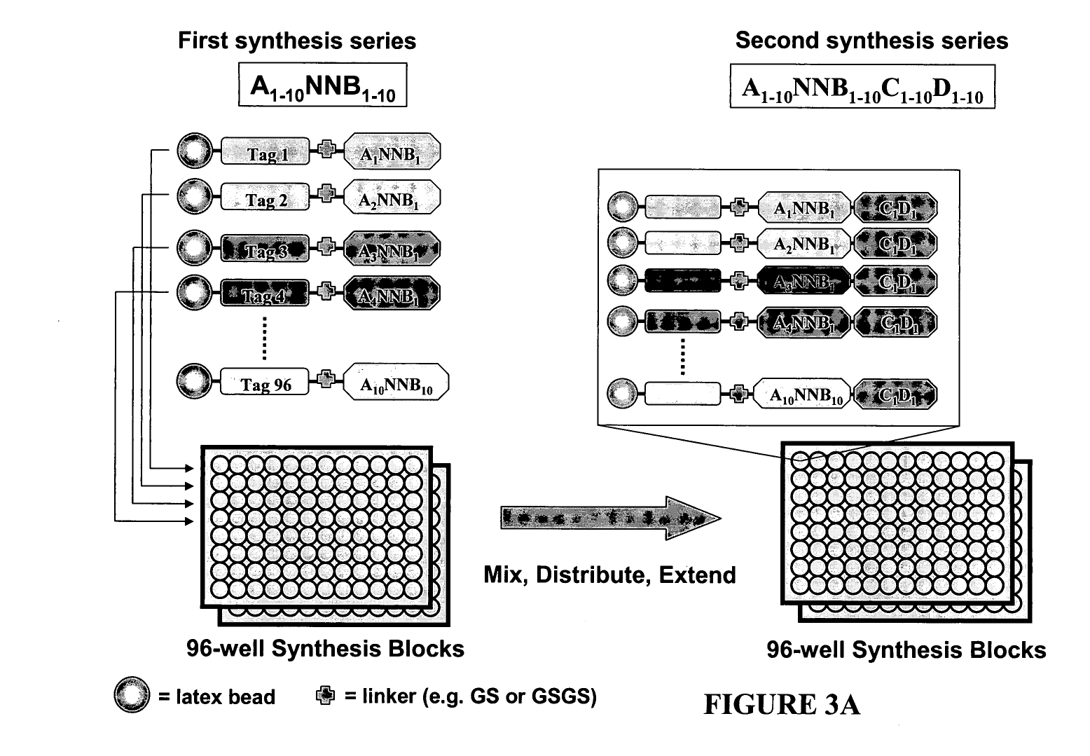

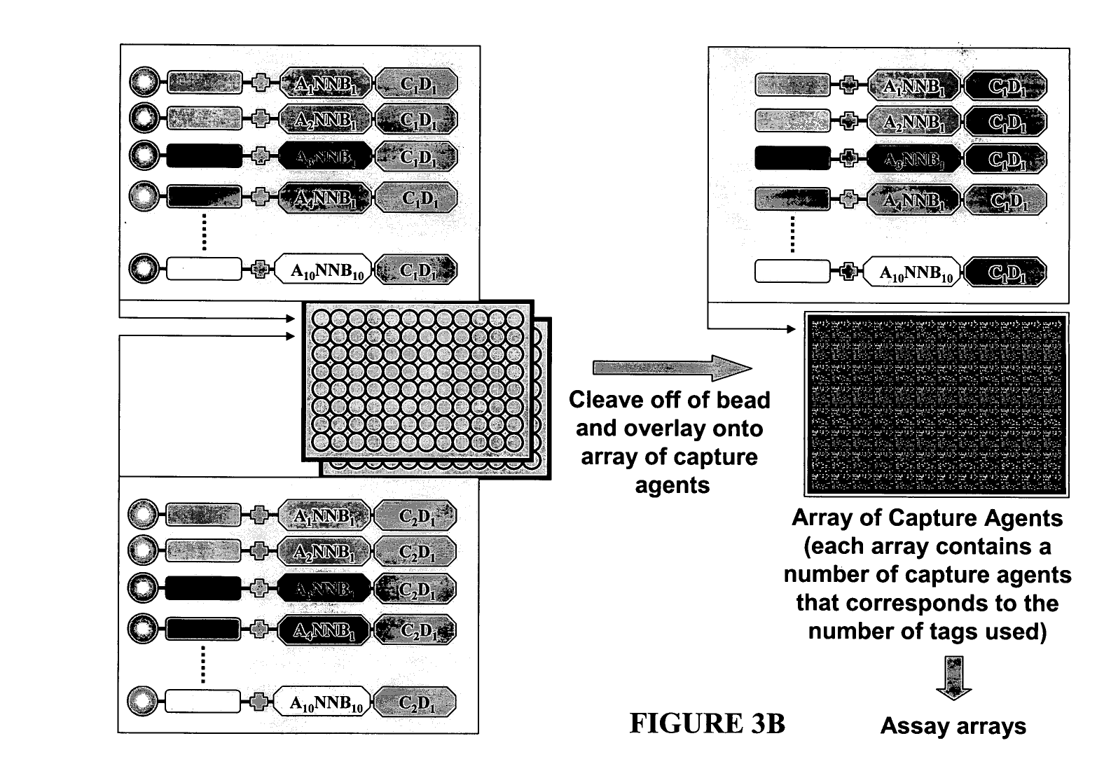

The mix is redistributed to a second synthesis block such essentially all of the combinations from the first block represented at each synthesis address (e.g. each well, tube etc) in the second block. The second block also is gridded out as a 10×10 or other suitable format such that 10 amino acids will be distributed at each of the third and fourth variable positions. Position C is synthesized by adding 10 amino acids to the synthesis grid as follows. Each row receives 1 amino acid, such that all positions in the row receive the same amino acid and each different row receives a different amino acid. (e.g. row 1 receives amino acid 1, row 2 receives amino acid 2 etc). Position D is synthesized by adding the 10 amino acids designated for position D to the grid where each column receives 1 amino acid, such that all positions in the column receive the same amino acid and each different column receives a different amino acid. (e.g. column 1 receives amino acid 1, column 2 receives amino acid 2 etc). The peptides synthesized are represented by the formula tag-linker-A-GS-B-C-D, where addresses on the grid can be represented as the series: tag-linker-A₁₋₁₀-GS-B₁₋₁₀-C₁-D₁, tag-linker- A₁₋₁₀-GS-B₁₋₁₀-C₁-D₂ . . . . A₁₋₁₀-GS-B₁₋₁₀-C₂-D₁ . . . A₁₋₁₀-GS-B₁₋₁₀-C₁₀-D₁₀ (sees FIGS. 3A and 3B).

The synthesized peptides are cleaved from the solid support and the collection of peptides in each well is transferred to an array of capture agents, where the number of capture agents in each array, b, is the same as the number of tags. Each capture agent array receives a collection of peptides of the formula A₁₋₁₀-GS-B₁₋₁₀-C_{y}-D_{z}, where y and z are independent integers between 1 and 10, representing the 10 possible amino acids at each of the C and D positions. The number of capture agent arrays is equal to the number of synthesized C-D combinations (10×10=100). The capture agent arrays bind specifically to the tags such that each unique tag binds to a unique capture agent in the array, thus peptides with different amino acids at the A and B positions are displayed by different capture agents. Thus, within each array each of the A-B combinations is addressed via the tag:capture agent interaction and within a single array all A-B combinations have the same amino acids at positions C and D.

The canvas of arrays of synthesized displayed HAHS polypeptides can be used for screening. For example, the canvas of HAHS polypeptides is incubated with a single chain antibody (ScFv). Specific binding of the antibody to HAHS polypeptides of the arrays is assessed, for example by staining, such as a stain that reacts with the constant chain of the ScFv. The staining indicates specific binding to one or more HAHS polypeptides. Screening of a collection of ScFvs with the canvas of arrays of synthesized displayed HAHS polypeptides can be used to isolate ScFvs which specifically bind to HAHS polypeptides and have little or no cross-reactivity with other HAHS polypeptides. The ScFvs and HAHS polypeptides which specifically bind can be further used as capture agents and binding partners as described herein. Methods can be used which generate collections of HAHS polypeptides, including large diverse collections of HAHS polypeptides. In one example of the method, collections of HAHS polypeptides are synthesized in an addressable format.

### 4. Assessment of Antigenicity

HAHS polypeptides can be assessed for antigenicity in vivo and/or in vitro. For example, HAHS polypeptides can be injected into a subject and then subsequently assessed for antibody response such as by assessing antibody titer and affinity of antibodies that recognize the injected HAHS polypeptide. HAHS polypeptides also can be assessed by their interactions with an antibody library such as a phage display antibody or antibody fragment library. The number of antibodies and the affinity of binding to HAHS polypeptides can be assessed. Additionally, antibodies can be obtained from subjects, such as a panel of antibodies and/or antisera collected from subjects. Such collections can be used to screen HAHS polypeptides for antigenicity.

In some cases, it can be desirable to identify HAHS polypeptides which are antigenic in one species but less antigenic in another. For example, it can be desirable to identify HAHS polypeptides which are antigenic in rodents but less antigenic in humans. Such assessments can be done empirically. For example, HAHS peptides can be assessed in vivo in a first subject, or in vitro with an antibody library from the first subject, as described above. The HAHS polypeptides also can be tested in a second subject either in vivo or using an in vitro screen. HAHS polypeptides are then identified which display a level of antigenicity in the first subject but a lower level in a second subject. Such comparisons can use assessments such as, but not limited to, assessments based on the titre of antibodies, raised, the number or type of antibodies binding the HAHS polypeptide, and the affinity of antibodies for the HAHS polypeptides or any other means known in the art for assessing antigenicity. Such assessments can be relative as compared to control peptides or other HAHS polypeptides.

### C. IDENTIFICATION OF CAPTURE AGENTS WHICH BIND HAHS POLYPEPTIDES

Capture agents are generated and/or selected that specifically bind the highly antigenic, highly specific polypeptides, thereby generating pairs of molecules. Each pair contains a capture agent which specifically and selectively binds to a highly antigenic highly specific polypeptide, designated as a binding partner for the pair. Pairs of capture agents and binding partners can then be used in applications such as addressable collections and capture systems. As noted, the polypeptide binding partners provided herein and the methods for generating such polypeptide binding partners provide polypeptides that are designed to be antigenic and thus antibodies or antibody fragments can be generated and/or selected as capture agents which specifically bind to the polypeptides.

Candidate capture agent—binding partner pairs can be identified by any method known to the art, including, but are not limited to, raising antibodies from exposure of a subject to one or more binding partner polypeptides, screening of an antibody or antibody fragment library with one or more polypeptides and any other method known to those of skill in the art for identifying pairs of molecules that bind with high affinity and specificity. The following discussion provides exemplary methods; others can be employed.

### 1. Raising Antibodies

Antibodies contemplated herein include polyclonal antibodies, monoclonal antibodies and binding fragments thereof. Polyclonal antibodies are employed where high affinity (avidity) is desired. Polyclonal antibodies are typically obtained by immunizing an animal and isolating the polyclonal antibodies produced by the animal.

For example, antibodies have traditionally been obtained by repeatedly injecting a suitable animal (e.g., rodents, rabbits and goats) with an antigen or antigen with adjuvant. If the animal's immune system has responded, specific antibodies are secreted into the serum. The antibody-rich serum (antiserum) that is collected contains a heterogeneous mixture of antibodies, each produced by a different B lymphocyte. The different antibodies recognize different parts of the antigen, and are thus a heterogeneous mixture of antibodies. A homogeneous preparation of antibodies can be prepared by propagating an immortal cell line wherein antibody producing B cells are fused with cells derived from an immortal B-cell tumor. Those hybrids (hybridoma cells) that are producing the desired antibody and have the ability to multiply indefinitely are selected. Such hybridomas are propagated as individual clones, each of which can provide a permanent and stable source of a single antibody (a monoclonal antibody) which is specific for the antigen of interest. The antibodies can be purified from the propagating hybridomas by any method known to those skilled in the art. Fragments of antibodies can be synthesized or produced and modified forms thereof produced.

In one exemplary embodiment, mice are immunized with a collection of polypeptide binding partners generated by the methods provided herein, for example as diphtheria toxin-6 mer polypeptide conjugates. The 6-mer has 2 non critical positions and 4 critical positions. The 2 non-critical positions of the 6-mer are adjacent to each other. The non-critical positions are not found at the ends of the polypeptide and thus are represented at two positions of positions 2, 3, 4 and 5. The 2 non-critical positions are chosen from S, G and Y. The remaining 4 critical residues are selected from the top 10 antigenic amino acids in table X: E, P, Q, N, F, H, T, K, L, and D.

Antibodies are raised against the collection of polypeptides. A library of hybridoma cells is then generated and clones are screened for their reactivity with individual polypeptides. Positive clones identify monoclonal antibodies which bind a selected polypeptide binding partner. Antibodies can be isolated by standard immunopurification techniques or by cloning methods such as by PCR with primers for conserved regions of the antibody structure.

Once an antibody is isolated, a corresponding binding partner (e.g., a HAHS polypeptide used in the generation of the antibody) can be conjugated to a molecule and/or biological particle, as described below, and screened against the antibodies isolated above to determine whether the antibodies retain the ability to specifically bind the polypeptide, thereby identifying a capture agent—binding partner pair.

### 2. Antibody Library Screening

Antibodies and antibody fragments also can be selected, for example, by screening a library, for antibodies which specifically bind to HAHS polypeptides. For example, a single chain antibody library can be constructed and screened against one more HAHS polypeptides to identify pairs of single chain antibodies and HAHS polypeptides. Phage display, protein expression library screening and antibody arrays as well as other screening methods well known in the art can be used to screen antibodies and antibody libraries for binding to HAHS polypeptides.

For example, to identify binding proteins using panning and phage display, hybridoma cells are first created either from non-immunized animals or animals (such as mice) immunized with a library of random epitopes or immunized with groups or libraries of HAHS polypeptides. The mice (or other immunized animals) are initially screened for high immunoglobulin (Ig) production and binding to one or more HAHS polypeptides. Ig production can be measured, for example, by ELISA assay of culture supernatants using an anti-IgG antibody (e.g. anti-mouse IgG to measure IgG produced in mice). Such assays can be performed in 96-well formats or any other suitable formats. Animals producing sufficient IgG with reactivity to HAHS polypeptides are then used to generate material for antibody libraries.

To produce a library, MRNA is isolated from spleenocytes or peripheral blood lymphocytes (PBLs). PCR and/or other amplification methods can be used to amplify conserved sequences in antibodies and antibody fragments. For example, functional antibody fragments can be created by genetic cloning and recombination of the variable heavy (V_{H}) chain and variable light (V_{L}) chain genes. The V_{H} and V_{L} chain genes are cloned by first reverse transcribing mRNA isolated from spleen cells or PBLs into cDNA. Specific amplification of the V_{H} and V_{L} chain genes is accomplished with sets of PCR primers that correspond to consensus sequences flanking these genes. The V_{H} and V_{L} chain genes are joined with a linker DNA sequence. A typical linker sequence for a single-chain antibody fragment (scFv) encodes the amino acid sequence (Gly₄Ser)₃. After the V_{H} -linker-V_{L} genes have been assembled and amplified by PCR, the products can be transcribed and translated directly or cloned into an expression plasmid. Cloned antibodies, such as in expression vectors suitable for phage display, are then expressed in vitro or in vivo and used for screening. Additional diversity can be introduced into phage display libraries by recombination and/or mutagenesis techniques such as error-prone PCR.

The phage library of antibodies and/or antibody fragments, is panned against one or more HAHS polypeptides and those which specifically bind are isolated. The bacteriophage that display antibodies and/or antibody fragments interacting with HAHS polypeptides can be isolated through washing and then enriched through multiple panning steps, resulting in a high population of phage displaying an antibody and/or antibody fragment that specifically bind an HAHS polypeptide. Such screening identifies pairs of antibodies and/or antibody fragments and HAHS polypeptides, for use as capture agents and binding partner pairs.

### 3. Engineered Capture Agents

Isolated and/or cloned antibodies and antibody fragment also can be used to design and construct additional capture agents. For example, variable regions from an antibody which binds an HAHS polypeptide can be used as a capture agent. Variable regions can be isolated by enzymatic or recombinant means. For example, immunoglobulin molecules can be cleaved with papain and/or pepsin, to produce Fab and F(ab′)₂ molecules containing 1 or 2 variable regions respectively. Similar molecules also can be constructed by recombinant means, such as by using PCR and primers to conserved regions within the light and heavy chains. The variable domains can be joined by a linker in a single chain to create single chain antibodies (ScFvs). Such domains can be joined covalently or non-covalently to other polypeptides and/or other domains from polypeptides, for example to add additional functionalities.

Capture agents also can be constructed from complementarity determining regions (CDRs). Recombinant means can be used to isolate the CDRs which are contained in the hyper variable loops of the antibody variable domain and are involved in antigen binding. Once isolated one or (up to all 6) of the CDRs can be cloned into a protein scaffold (see for example, Skerra (2000) J. Mol. Recognit. 13:167-187). Protein scaffolds include any polypeptide in which the CDRs can be placed and maintain binding to an antigen. Exemplary protein scaffolds include antibody and antibody fragments, fibronectin, protease inhibitors such as bovine pancreatic trypsin inhibitor, human pancreatic trypsin inhibitor, and tendmsitat, helix bundle proteins including natural and engineered structures such as the “Z” domain, lipocalins, knottins, and enzymes such as glutathione S-transferase, thioredoxin, and triose phosphate isomerase.

Capture agents also can be constructed as protein fusions with antibodies or fragments thereof that bind HAHS polypeptides. For example, a tag can be added for purification, identification or for localization. Such tags include His₆ and myc tags and GST fusions for purification, (nuclear, membrane, secretion), labels for detection such as fluorescent proteins and enzymes such as luciferase, β-galactosidase, and alkaline phosphatase, and localization sequences such as for membrane localization, secretion and organelle localization such as nuclear and chloroplast localization.

### D. PRODUCING MOLECULED TAGGED WITH HAHS POLYPEPTIDE BINDING PARTNERS

HAHS polypeptides for use as binding partners can be conjugated to molecules and/or biological particles for example for use in addressable collections and capture systems. HAHS polypeptides can be conjugated to molecules and/or biological particles by any means known in the art such as those described herein, including, but not limited to, recombinant means and chemical linkages, such that the binding partners still retain the ability to specifically bind capture agents. The conjugation can be direct or indirectly via a linker. Collections of binding partners can be associated with collections of molecules and/or particles to create binding partner-tagged libraries. For example, HAHS polypeptides can be encoded by nucleic acid molecules which can be joined with nucleic acid molecules encoding another polypeptide to create tagged-polypeptides such as described herein. A collection of nucleic acid molecules encoding HAHS polypeptides can be used to create a tagged library of molecules.

Molecules and/or particles can be tagged with binding partners using covalent or non-covalent interactions to conjugate the binding partner and the molecule and /or biological particle. The conjugation can be effected by any method known to those skilled in the art, such as chemically, by recombinant expression of a fusion protein, via a linker molecule and by any combination thereof. For example, the conjugates can be produced by chemical conjugation, such as via thiol linkages, to produce covalent bonds, ionic linkages or linkages via other chemical interactions, such as van der Waals interactions, hydrophobic interactions and other such interaction. The resulting conjugate, however, should be sufficiently stable for subsequent use of the tagged molecule and/or particle. For example, upon binding of a binding partner to a capture agent, the linked molecule and/or biological particle is retained.

For convenience and exemplification, the conjugates provided can be represented by the formula:
(BP)ₛ-(L)ₜ-(M)ₚ
L is an optional linker, BP is binding partner, M is molecule and/or biological particle and BP is linked either directly or indirectly via one or more linkers to M such that the resulting conjugate remains conjugated when bound to a capture agent. In the formula, t is 0 or an integer of 1 up to x; s and p, which are the same or different, are integers of 1 up to y; and x and y, which are the same or different, are generally 1 or 2, but can be 2, 3, 4, 5, 6 or more as long as the resulting conjugate binds to a capture agent. For example, where M is a biological particle such as a cell, each cell can have a plurality of receptors or other surface molecules to which a binding partner binds. In such instances, p can vary from conjugate to conjugate and also can not be readily ascertained. The stoichiometry of each conjugate is not critical to practice of the method. Stoichiometry can be selected and controlled by methods known to those of skill in the art, such as empirically or by selecting appropriate concentrations of the binding partner and moiety to be tagged.

### 1. Chemical Conjugates

Any chemical or biological reaction known to those of skill in the art that results in the formation of a linkage between a molecule and/or biological particle and a polypeptide binding partner can be used to form conjugates (see for example, U.S. application Ser. No. 10/699,113 and International application Serial No. PCT/US03/34747). Molecules and biological particles can be coupled to binding partners via direct or indirect linkages, including, but not limited to, covalent, ionic, hydrophobic and van der Waals interactions, as long as the linkage is stable enough to be maintained upon exposure of the conjugate to subsequent manipulations, such as binding to a binding protein or capture agent. Molecules, such as proteins, and biological particles contain several reactive groups, including, but not limited to, amino, hydroxyl, sulfhydryl, phenolic and carboxyl groups, that can be used as sites of chemical cross-linking to produce novel polymeric structures. Exemplary linkages that are suitable for the formation of chemically linked conjugates include disulfide bonds, thioether bonds, hindered disulfide bonds, and covalent bonds between free reactive groups, such as amine and thiol groups.

Any interaction between molecules and/or biological particles and polypeptide binding partners, including, but not limited to, polypeptide:polypeptide, polypeptide:nucleic acid, polypeptide:lipid, and polypeptide:small molecule interactions can be used for the formation of the conjugates. For example, a conjugate can be prepared from the reaction of a polypeptide binding partner, such as designed by the methods provided herein and an antibody or fragment thereof which recognizes the polypeptide binding partner.

Chemical conjugation also can be effected by any method known to those of skill in the art including, but not limited to alteration in environmental conditions, such as alteration in temperature, pH and buffer components, and/or the addition of a compound or molecules known to catalyze the formation of a chemical linkage, such as a cross-linking reagent. For example, cross-linking reagents including, but not limited to, heterobifunctional, homobifunctional and trifunctional reagents, can be used to introduce, produce or utilize reactive groups, such as thiols, amines, hydroxyls and carboxyls, on one or both of the molecules or biological particles or binding partners, which can then be contacted to a target molecule and/or biological particle or binding partner containing a second reactive group, such as a thiol, amine, hydroxyl and carboxyl, to form a chemical linkage between the molecule and/or biological particle or binding partner. These reagents can be used to directly or indirectly, such as through a linker, conjugate a molecule and/or biological particle to a binding partner. Generally, cross-linking reagents have two reactive groups connected by a flexible spacer arm. The reagents differ in their spacer arm length, cleavability, solubility and reactive groups, and can be selected to alter a characteristic of the conjugate complex, such as the solubility, stearic hinderance and permeability. Some cross-linking reagents (i.e., homobifunctional cross-linkers) have the same reactive groups at both ends, others (i.e., hetero-bifunctional cross-linkers) have different reactive groups at the ends and some cross-linkers contain additional functional groups to allow the cross-linker molecule to be labeled. Additionally, some cross-linking reagents (i.e., trifunctional cross-linkers) have three reactive groups to make trimeric complexes.

Cross-linking reactions involving molecules and binding partners, such as proteins, are generally reactive group reactions, such as side chain reactions, and are nucleophilic, resulting in a portion of the end of the cross-linker being displaced in the reaction (the leaving group). Nucleophilic attack is dependent on the pH, temperature and ionic strength of the cross-linking buffer. For example, when the buffer is one to two pH units below the pKₐ of the reactive group, such as a side chain, the species is highly protonated and is most reactive. One to two Ph units above the pKₐ, the species is not protonated and not reactive. The majority of molecules and binding partners, such as proteins, have reactive groups, such as primary amines and free sulfhydrals, available at the surface or terminus of the molecules or binding partner. These are the two most commonly used groups in molecular cross-linking strategies. Cross-linking strategies also can use carbohydrates, carboxyls or other reactive functional groups.

Many factors are considered to obtain optimal cross-linking for a particular application. Factors that affect molecular folding, such as protein folding, (e.g., pH, salt, additives and temperature) can alter conjugation results. Other factors such as molecule or binding partner concentration, cross-linker concentration, number of reactive functional groups available, cross-linker spacer arm length, and conjugation buffer composition should also be considered.

### 2. Fusion Proteins

Fusion proteins are exemplary of conjugates provided herein. A fusion protein can contain, for example, a polypeptide of interest and a binding partner. The binding partner can be designed and constructed using the methods provided herein. Exemplary polypeptides for use as binding partners in fusion proteins described herein can, for example, be short polypeptide molecules, such as molecules with at least 5, 6, 8, 10, 15, 20 or more amino acid residues. Exemplary HAHS polypeptides for use as binding partners in fusion proteins are given in SEQ ID NOs: 1-911.

Fusion proteins can be produced by recombinant expression of nucleic acids that encode the fusion protein. The formation of a fusion protein involves the placement of two separate coding sequences, such as genes or nucleotides sequences, one encoding the displayed molecule and the second encoding the binding partner, in sequential order in an appropriate cloning vector. Methods for creating an expression vector containing the displayed molecule and the binding partner are well known to those of skill in the art (see, e.g., Sambrook et al. (1989) Molecular Cloning: A Laboratory Manual, Clod Spring Harbor Laboratories, Cold Spring Harbor, N.Y.). Additional methods for the formation of a fusion protein conjugate include, but are not limited to ligation of sequences resulting in linear tagged cDNA molecules; primer extension and PCR for binding partner incorporation; insertion by gene shuffling; recombination strategies; incorporation by transposases; and incorporation by splicing.

### 3. Linkers

Any linker known to those of skill in the art for preparation of conjugates can be used herein. These linkers are typically used in the preparation of chemical conjugates. Peptide linkers can be incorporated into fusion proteins. Linkers can be any moiety suitable to associate a molecule and/or biological particle and a binding partner. Such linkers and linkages include, but are not limited to, peptidic linkages, amino acid and peptide linkages, typically containing between one and about 60 amino acids, more generally between about 10 and 30 amino acids, and chemical linkers, such as the heterobifunctional, homobifunctional and trifunctional cross-linkers described above. Other linkers include, but are not limited to, acid cleavable linkers, such as bismaleimideothoxy propane, acid labile-transferrin conjugates and adipic acid dihydrazide, that would be cleaved in more acidic intracellular compartments; cross linkers that are cleaved upon exposure to UV or visible light and linkers, such as the various domains, such as C_{H}1, C_{H}2, and C_{H}3, from the constant region of human IgG₁ (see, Batra et al. (1993) Molecular Immunol. 30:379-386).

Chemical linkers and peptide linkers can be inserted by covalently coupling the linker to the binding partner and displayed molecule. The heterobifunctional agents, described above, can be used to effect such covalent coupling. Peptide linkers also can be linked by expressing DNA encoding the linker and displayed molecule as a fusion protein as described above. Flexible linkers and linkers that alter the characteristics, including, but not limited to the solubility, stearic hinderance, overall charge, pH stability and cleavability, of the conjugated molecules are contemplated for use, either alone or with other linkers are contemplated herein.

Linkers also can include intermediate molecules such as any solid or semisolid or insoluble support to which a binding partner can be attached. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials to which binding partners and molecules and/or biological particles can be attached. A intermediate molecule can be of any geometry, such as particulate. When particulate, typically the particles have at least one dimension in the 5-10 mm range or smaller. Such particles, referred collectively herein as “beads,” are often, but not necessarily, spherical. Such reference, however, does not constrain the geometry of the matrix, which can be any shape, including random shapes, needles, fibers, and elongated. Roughly spherical “beads,” particularly microspheres that can be used in the liquid phase, are contemplated.

The intermediate molecules can include additional components, such as magnetic or paramagnetic particles (see, e.g., Dyna beads® (Dynal, Oslo, Norway)) for separation using magnets, as long as the additional components do not interfere with the methods and analyses herein. Such intermediate molecules also can contain identifiers such as electronic, chemical, optical or color-coded labels.

Binding partners can be bound or conjugated to beads by any method known in the art. For example, binding partners can be bound by adhesion to the intermediate molecule or by association of charged groups between them. Binding partners also can be covalently attached to the intermediate molecules by a cross-linker, chemical conjugation or by a chemical linkage such as described herein. Biological molecules and/or particles also can be attached to the intermediate molecules using non-covalent interactions including electrostatic and hydrogen bonds, covalent interactions or a combination thereof. Such attachments can include adhesion and charge association, as well as covalent binding, such as cross-linking, chemical conjugation or chemical linkage.

Single molecules of a binding partner or multiple molecules of a binding partner can be bound or conjugated to the intermediate molecule. Similarly, single biological molecules and/or particles or multiple biological molecules and/or particles can be bound or conjugated to the intermediate molecule.

### 4. Tagged Libraries

The methods and compositions provided herein can be used to generate a collection of HAHS polypeptide binding partners for use in constructing a tagged library. The methods can be used to design a collection of dissimilar binding partner tags such that there is a greater affinity between paired capture agents and binding partners than for other HAHS polypeptides or capture agents in a collection. Thus a library can be sub-divided into sets each tagged with a unique and specific HAHS polypeptide binding partner.

Libraries of binding partner-tagged molecules can include, but are not limited to, nucleic acid libraries, polypeptide libraries and chemical libraries. HAHS polypeptide tags can be used in place of, or in addition to other types of tags, such as optically encoded tags, RF-tags, mass tags and color tags for tagging libraries. HAHS polypeptides can be conjugated to the library members during or after synthesis of the library members.

In one embodiment, tagged libraries are produced by attaching, directly or indirectly, a nucleic acid molecule encoding a binding partner designed by methods provided herein, to members of the library, such that when the library is translated to produce a library of polypeptides, the binding partner (containing the HAHS polypeptide) is in frame with molecule to be tagged. Cloning of nucleic acids encoding binding partners and their attachment to a library such as a cDNA library can be accomplished using a variety of available methods including, but not limited to, ligation into plasmids containing nucleic acid sequences encoding binding partners, ligation of linear nucleic acids encoding binding partners, primer extension and PCR methods, gene shuffling, recombination, transposase methods, and splicing.

Collections of dissimilar binding partner tags generated by methods such as described herein can be used with methods for effecting even distribution. In many applications of library construction, even distribution of binding partner tags is advantageous. For example, an even distribution of the binding partners among tagged molecules allows for the control of the diversity of the tags among the loci of an addressable array. Methods for effecting even distribution sufficient for use of the capture systems have been described (see, e.g., published International PCT application No. WO 02/06834; published U.S. applications Serial Nos. US20020137053 and US20030143612; U.S. application Ser. No. 10/699,088 and International PCT application serial No. PCT/US03/34821).

In another embodiment, chemical libraries tagged with HAHS polypeptides are produced. Such libraries can include, but are not limited to, small molecule libraries, natural product libraries, oligonucleotide libraries, nucleic acid libraries and combinatorial chemistry libraries. HAHS polypeptide tags can be unique to each chemical structure within the library. Alternatively, families of related structures can be tagged with a unique HAHS polypeptide. In one embodiment, HAHS polypeptides are used to tag chemical libraries in a solid phase synthesis method. The HAHS polypeptides are conjugated to beads for use in chemical library synthesis. The beads can be cleaved from the synthesized chemicals at the completion of the synthesis protocol or they can remain associated with the synthesized molecules and used, for example, to further sort and display the library for screening. HAHS tags also can be used to sort libraries after synthesis, for example, to deconvolute mixtures of library members. HAHS tags also can be used for purification of library members, for example by contacting them with capture agents to which the HAHS polypeptides bind.

### E. USE OF BINDING PROTEINS IN CAPTURE SYSTEMS

The collections of highly antigenic highly specific polypeptides and the methods for generating such collections can be used to construct addressable collections and capture systems. Such collections and systems can be used to display biological molecules and particles. They also can be used to screen for and assay biological function and effect.

### 1. Preparation of Capture Systems

Capture systems are made up of capture agents and binding partners.

The binding partners specifically bind to capture agents to produce the capture systems. The capture systems can be constructed using polypeptide binding partners constructed by the methods provided herein to display biological molecules and particles for further assays.

Capture systems rely upon the use of the capture agents and binding partners that contain the sequence of amino acids to which the capture agent or a binding portion thereof specifically binds. The methods provided herein can be used to generate HAHS polypeptides, such as for example SEQ ID NOs: 1-911, for use as binding partners in capture systems.

The addressable capture agent collections, such as a positionally addressable array, contains a collection of different capture agents that bind to binding partners. Each locus or address contains a single type of capture agent that binds to a single specific binding partner. Tagged molecules, such as biological molecules and particles are contacted with the collection of capture agents in an array, under conditions suitable for complexation with the capture agent via the binding partner associated with the biological molecule or particle. As a result, molecules and particles are sorted according to the binding partner each possesses and displayed.

a. Preparation of Binding Partners

As described above, HAHS polypeptides cam be used as binding partners to tag biological molecules and particles. The methods provided herein can be used to generate collections of binding partners to which capture agents, such as antibodies and antibody fragments bind.

HAHS polypeptide binding partners can be encoded by a nucleic acid that is used to construct binding partner tagged molecules by recombinant means.

The nucleic acid construct permits expression of the encoded tagged polypeptide. Libraries of molecules can be tagged with peptide binding partner tags. The number of peptides chosen for tagging and the number of molecules to be tagged determines the diversity of the tags in the library.

In many applications even distribution of tags is advantageous. For example, an even distribution of the tags among tagged molecules allows for the control of the diversity of the tags among the loci of an addressable array. Ideally, the diversity of tags of a locus is about 1, but on the average can be more than 1, up to about 100, 50, 25, 10, 5, 1.5 or 1.1 .

An even distribution of tags permits a higher diversity of tagged molecules at each locus. The diversity of tagged molecules at each locus can be 10², 10³, 10⁴, 10⁵, 10⁶ or greater. If there is an even distribution of tags, then the diversity of molecules at each locus is substantially the same, generally within 1, 0.5, 0.1 order of magnitude. If the tags, however, are not evenly distributed, then the same tagged molecules will be at a plurality of loci in a capture system. Once the tags are evenly distributed, the diversity of tagged molecules at each locus can be selected or adjusted as desired and depends upon the application.

Nucleic acid encoding a HAHS polypeptide binding partner also can include sequences of nucleotides that can aid in unique or convenient priming, such as for PCR amplification, or can encode amino acids that confer desired properties, such as trafficking signals, detection, solubility alteration, facilitation of purification or conjugation or other functions or provide other functions. For example, in embodiments in which candidate components are subcloned into a panel of vectors each containing an HAHS binding partner, these additional sequences also can included in the vector.

For certain applications, HAHS polypeptide binding partners do not have to be fused to biological molecules or particles. It is possible to prepare binding partners that are encoded as separate peptides that are physically or otherwise associated or linked with the candidates. For example, chemical conjugation or molecular interactions such as dimerization, can be used to associate binding partners with the candidate molecules to be associated with the capture system.

The HAHS polypeptide binding partners also can be incorporated as part of a larger polypeptide, for example as an N-terminal or C-terminal sequence of the polypeptide, or within the polypeptide sequence. Such incorporation can be as a fusion protein, by fusing the nucleic acid sequence encoding the peptide binding partner with a nucleic acid molecule encoding a polypeptide of interest. The sequences are fused in-frame to generate a single polypeptide with the peptide binding partner sequence incorporated.

b. Capture Agents

A capture agent is a molecule that has an affinity for a defined sequence of amino acids or other site on another molecule, such as a ligand, or for purposes herein a binding partner. Capture agents include any agent that specifically binds with sufficient affinity to a binding partner, for further use in the resulting capture systems. As described herein, capture agents can be generated and/or selected which bind HAHS polypeptides for use as capture agent-binding partner pairs. Exemplary capture agents for use with HAHS polypeptide binding partners include, but are not limited to, antibodies and antibody fragments including Fab, Fab′, F(ab)₂, single chain antibodies (ScFvs), Fv, dsFv, diabodies, bispecific antibodies and binding regions of antibodies such as complementarity determining regions (CDRs) in protein scaffolds.

Capture agents, such as antibodies and antibody fragments, that bind to polypeptide binding partners designed by the methods herein can be generated by any methods known in the art. For example, antibodies and antibody fragments for use as capture agents can be generated by raising antibodies against polypeptide binding partners such as described herein. Capture agents that bind polypeptide binding partners also can be selected from a library such as an antibody library, for example, a library of single chain antibody fragments.

The methods rely upon the ability of capture agents to specifically bind to the binding partners. The specificity of each capture agent for a particular binding partner is known or can be readily ascertained, such as by arraying the capture agent so that all of the capture agents at a locus have the same specificity. Therefore, candidates binding to each locus based on their binding partner can be identified.

Capture agents can be positionally addressed. Alternatively, each can be addressed by associating them with unique identifiers, such as by linkage to optically encoded identifiers, including colored beads or bar coded beads or supports, or linked to electronic identifiers, such as by providing microreactors with electronic tags or bar coded supports or colored identifiers or other such addressing methods that can be used in place of physically addressable arrays. For example, each capture agent can be bound to a support matrix associated with a color-coded tag (i.e. a colored sortable bead) or with an electronic tag, such as a radio-frequency tag (RF), such as IRORI MICROKANS® and MICROTUBES® microreactor.

### 2. Preparation of Capture Agent Arrays

By reacting a collection of capture agents with polypeptide binding partner-labeled molecules, so that the binding partners bind to their cognate capture agent, capture systems are prepared. Such capture systems have been previously described (see, e.g., U.S. application Ser. No. 09/910,120, published as U.S. application Serial No. 20020137053; published International PCT application No. WO 02/06834; and U.S. application Ser. No. 10/341,226; published as U.S. application Ser. No. 20030143612; U.S. application Ser. Nos. 10/699,113, 10/699,114 and 10/699,088, and International Application Nos. PCT/US03/34821, PCT/US03/34747, and PCT/US03/34693.

Each locus of a collection of capture agents contains a multiplicity of capture agents, such as antibodies with a single specificity. In solid phase embodiments, in which the capture agents are displayed as loci, each locus is of a size suitable for detection. Loci can be on the order of 1 to 300 microns, typically 1 to 100, 1 to 50, and 1 to 10 microns, depending upon the size of the array, target molecules and other parameters. Generally the loci are 50 to 300 microns. In preparing the arrays, a sufficient amount is delivered to the surface to functionally cover it for detection of proteins having the desired properties. Generally the volume of antibody-containing mixture delivered for preparation of the arrays is a nanoliter volume (1 up to about 99 nanoliters) and is generally about a nanoliter or less, typically between about 50 and about 200 picoliters. This is very roughly about 10 million to 100,000 molecules per locus, where each locus has capture agents that recognize a single bp-tag. The size of the array and each locus is such that positive reactions in the screening step can be imaged, generally by imaging the entire array or a plurality thereof, such as 24, 96, or more arrays, at the same time.

A support, such as KODAK paper plus gelatin, plastic or other suitable matrix can be used, and then ink jet and stamping technology or other suitable dispensing methods and apparatus, are used to reproducibly print the arrays. The arrays are printed with, for example, a piezo or inkjet printer or other such nanoliter or smaller volume dispensing device. For example, arrays with 1000 loci can be printed. A plurality of replicate arrays, such as 24 or 48, 96 or more can be placed on a sheet the size of a conventional 96 well plate.

Capture agents also can be linked to beads or other particulate supports that are associated with an identifier. For example, the capture agents are linked to optically encoded microspheres, such as those available from Luminex, Austin Tex., that contain fluorescent dyes encapsulated therein. The microsphere, which encapsulate dyes, are prepared from any suitable material (see, e.g., International PCT application Nos. WO 01/13119 and WO 99/19515; see description below), including styrene-ethylene-butylene-styrene block copolymers, homopolymers, gelatin, polystyrene, polycarbonate, polyethylene, polypropylene, resins, glass, and any other suitable support (matrix material), and are of a size of about a nanometer to about 10 millimeters in diameter. By virtue of the combination of, for example two different dyes at ten different concentrations, a plurality microspheres (100 in this instance), each identifiable by a unique fluorescence, are produced.

a. Immobilization and Activation

Numerous methods have been developed for the immobilization of proteins and other biomolecules onto solid or liquid supports. Among the most commonly used methods are absorption and adsorption or covalent binding to the support, either directly or via a linker, such as the numerous disulfide linkages, thioether bonds, hindered disulfide bonds, and covalent bonds between free reactive groups, such as amine and thiol groups, known to those of skill in art.

To effect immobilization, a solution of the protein or other biomolecule is contacted with a support material such as alumina, carbon, an ion-exchange resin, cellulose, glass or a ceramic. Fluorocarbon polymers have been used as supports to which biomolecules have been attached by adsorption. Methods for attaching biological molecules, including proteins and nucleic acids, to solid supports include but are not limited to, methods introducing free amino or carboxyl groups onto a silica support, modification of a polymer surface through the successive application of multiple layers of biotin, avidin and extenders, photoactivation methods, covalent binding to chemically activated solid matrix supports, directly linked to the matrix support or linked via a linker. The activation and use of supports are well known and can be effected by any such known methods (see, e.g., Hermanson et al. (1 992) Immobilized Affinity Ligand Techniques, Academic Press, Inc., San Diego). Exemplary linkages also include direct linkages effected by adsorbing the molecule or biological particle to the surface of the support.

b. Stabilization of Capture Agents and Polypeptide Binding Partners

As noted, the interactions between the capture agents and bp-tags are designed or selected to be of relatively high affinity and specificity. Any interaction, including, but are not limited to, hydrophobic, ionic, covalent and van der waals and combinations thereof is contemplated, as long as it meets the criteria of affinity and specificity.

Generally the interaction between the capture agent and binding partner is reversible, such as the interaction between an antibody and an epitope, and has an association constant sufficient for detection of subsequent binding events between the resulting capture system and other moieties.

Capture agents can be modified following the specific affinity interaction, such as by crosslinking between the bp-tag and the capture agent. For example, covalent cross-linking reagent (through chemical, electrical, or photoactivatable means) can be used to fix or stabilize interactions between proteins.

### 3. Screening

Collections of molecules and/or biological particles can be screened using HAHS polypeptides in capture systems, such as described herein, or in any other screening means know in the art. In preparation for screening, collections of molecules and/or biological particles can be generated and tagged with HAHS polypeptides. Such tagged molecules and/or particles can be displayed for example on a solid support, for example, through interactions with capture agents. The collections can then be screened for functions or effects of interest.

For example, interactions of HAHS binding partners and capture agents can be used to display and analyze biological particles, including, but not limited to, whole cells, eukaryotic and prokaryotic cells and fragments or organelles thereof or protein complexes; viruses, such as a viral vector or viral capsids with or without packaged nucleic acid; phage, including a phage vector or phage capsid, with or without encapsulated nucleotide acid; liposomes, other micellar agents or other packaging particles; and other such biological materials. Functions and effects on displayed biological molecules and particles can be assessed and can be used for a variety of purposes including, but not limited to, drug screening and interaction assessment. For example, in drug screening, a displayed interaction is known and perturbations are screened to identify candidate compounds and/or conditions that modulate the interaction among components of the target interaction. Alternatively, capture systems can be used to assess unknown molecular and/or biological particle interactions where an effect of a perturbation on a specific interaction or specific events is predetermined or preidentified, and any effect of the perturbation on unknown interactions or events can be used to identify the interaction or events in question. Examples of functions and effects that can be assessed with capture systems employing HAHS polypeptide binding partners include, but are not limited to, gene expression; DNA transcription; RNA translation; DNA and RNA synthesis products and intermediates; nucleic acid sequencing; protein sequencing; transfection; protein and peptide synthesis products and intermediates; enzyme activity analysis; antibody-antigen interactions; antibody specificity; protein or nucleic acid mutagenesis; DNA and RNA purification; nucleic acid hybridization; recombination processes; binding affinity assays; drug screening; protein interaction; cell morphology; signal transduction; complexation; membrane translocation; electron transfer; conversion of a reactant to a product via a catalytic mechanism; chaperoning of compounds inter- and intracellularly; fusion of liposomes to membranes; infection of a foreign pathogen into a host cell or organism, such as a virus (HIV, influenza virus, polio virus, adenovirus, etc.) or bacteria (Escherichia coli, Pseudomonas aeruginosa, Salmonella enteritidis, etc.); initiation of a regulatory cascade; detoxification of cells and organisms; and cell replication and division.

### 4. Combinatorial Synthesis of Tagged Libraries

Provided herein are methods for synthesizing collections of molecules in an addressable format. The methods are flexible for collection size and include preparation of small and large collections, including large diverse collections of molecules, addressably formatted and displayed suitable for screening and other assays.

As described herein, such methods can be used to synthesize collections of HAHS polypeptides. The methods provided herein can also be used to generate collections or libraries, in particular tagged libraries of molecules. For example, the methods can be used to generate tagged combinatorial libraries of small molecules, nucleic acids, polymers, including biopolymers and other types of combinatorial collections of molecules.

The methods utilize an addressable format provided by a collection of pairs of tags and capture agents. A collection contains pairs of molecules such that each tag binds a unique capture agent in the collection and each capture agent binds a unique tag in the collection. The total number of tag:capture agent pairs in a collection is designated “b.” In one embodiment, the collection of tags and capture agents for use with the methods include HAHS polypeptide tags and capture agents which bind the HAHS polypeptide tags.

The tags are used as a starting material for synthesis. One terminus of each tag is linked to a solid support, for example a latex bead. Such linkages can include optionally a first linker between the solid support and the tag and optionally, a second linker between the tag and the synthesis product. The other terminal group of the tag or second linker is used as the starting point for synthesis

The tags are gridded out or otherwise addressed for synthesis such that each tag occupies a unique address. For example, a microtiter plate is used as a synthesis block with unique tags conjugated to beads in each well. The tag-bead conjugates can be distributed to the wells or for example, beads can be distributed to all wells and each tag added to a different well and then conjugated. In another example, tags can be physically linked to a solid support and arranged in a grid. Any method known in the art can be used for addressing tag-solid support conjugates so long as the address for each tag is known or can be determined.

Molecules are synthesized on the tags using the tag or second linker as a starting material or alternatively, conjugating a starting molecule. For example, for small molecule synthesis a pharmacophore, as a starting molecule, is conjugated to the tag or second linker. For polymer synthesis, a starting monomer can be conjugated to the tag or second linker.

Molecules to be synthesized contain variable positions and optionally, additional fixed positions. Fixed positions refers to positions where all of the molecules to be synthesized have the same substituent at a given position; variable positions refers to positions where each molecule to be synthesized will not receive the same substituent but will receive one of a set of substituents designated for that position.

The first round of synthesis generates molecules with two variable positions and optionally any number of fixed positions.

For each variable position, a set of substituents is chosen, each set represents all of the possible substituents to be added at that position. The number of substituents chosen for use in synthesizing the two positions is set by the total number of available tag:capture agent pairs, b, such that such that b=X_{A}×X_{B} where X_{A} and X_{B} are the number of substituents in the set of substituents for each of the variable positions to be synthesized in the first round. The first round of synthesis generates collections of molecules where each unique tag now has a unique contains a unique combination of substituents at the two variable positions.

A second round of synthesis is initiated by mixing all the tagged synthesized molecules of the first round together and distributing them to a grid or otherwise divided synthesis container of b positions. The second round of synthesis adds an additional two variable positions of substituents, and optionally an additional number of fixed positions. The number of substituents chosen for use in synthesizing the two positions is set by the total number of available tag:capture agent pairs, b, such that such that b=X_{C}×X_{D} where X_{C} and X_{D} are the number of substituents in the set of substituents for each of the variable positions to be synthesized in the first round.

The second round of synthesis results in tagged molecules with a unique combination at two additional positions, such that each unique combination from the first round of synthesis has been extended with each unique combination of substituents in the second round. The variable positions synthesized in the first round are identifiable by the tags, since each unique combinations of substituents added in the first round is linked to a unique tag. The variable positions added in the second round are identifiable by their position in the second round synthesis; each address represents a unique combination of substituents added in the second round.

At the completion of synthesis, tagged molecules can be cleaved from the solid support if necessary. The tagged molecules are sorted by incubating them with the corresponding b number of capture agents, each binding a unique tag. Capture agents can be addressable by positional array or by virtue of a second tag such as an electronic, chemical, optically or color-coded bead, attached to each capture agent.

Molecules synthesized at each address in the second round synthesis are incubated with separate collection of addressed capture agents, such that there are b collections of addressed capture agents, each containing the same capture agents. For example, a canvas of b capture agent arrays is used where molecules from each address at the second round are incubated with a separate array on the canvas. Such distributions generate collections of capture agents, each collection displaying a subset of the synthesized molecules and together displaying the full set of synthesized molecules. Each collection of capture agents displays molecules with a unique combination of substituents added in the second synthesis round and the full assortment of possibilities of substituents added in the first synthesis round. The displayed collections of synthesized molecules can be used for screening and other functional assays.

### F. KITS

HAHS polypeptides described herein can be used in combinations of chemical and/or biological reagent(s), including, but not limited to collections of binding partners, collections of binding partners and capture agents, including binding partners and/or capture agents linked to solid supports, and conjugated with reagents such as plasmids and resins. Kits containing such reagents in packaged form, optionally including instructions for use thereof, also are provided. The instructional information typically can be in printed form, but also can be in an electronic or computer readable format on a computer readable medium or on the internet, such as, but not limited to, CD-ROM disks (CD-R, CD-RW), DVD-RAM disks, DVD-RW disks, floppy disks and magnetic tape.

For example, HAHS polypeptides can be supplied as a kit for tagging libraries. Such kits can include oligonucleotides which encode a collection of HAHS polypeptides, and/or the sequences of such oligonucleotides. The kits also can include plasmids or other DNA molecules to which the oligonucleotides encoding HAHS polypeptides are linked.

HAHS polypeptides also can be supplied as a kit for capture systems, for example, self-assembled arrays such as are described in U.S. application Ser. No. 10/699,113 and International Application Serial No. PCT/US03/34747. Such kits can include oligonucleotides which encode a collection of HAHS polypeptides, and/or the sequences of such oligonucleotides. Such kits also can include crosslinking reagents, such as described herein. Such kits also can include collections of beads or other particulate supports to which one or more HAHS polypeptides are linked. Such kits also can optionally include capture agents which bind HAHS polypeptides.

Kits can be used for purification, such as by tagging molecules and/or particles with one or more HAHS polypeptides and using one or more capture agents which bind the HAHS polypeptides linked to a solid support such as alumina, carbon, an ion-exchange resin, cellulose, glass, ceramic and fluorocarbon polymers.

Kits also can include instructional information for methods described herein for generating HAHS polypeptides. Such instructional information can be in an electronic or computer readable format on a computer readable medium or on the internet, such as, but not limited to, CD-ROM disks (CD-R, CD-RW), DVD-RAM disks, DVD-RW disks, floppy disks and magnetic tape. Instructional information also can include printed form instructions for generating HAHS polypeptide sequences.

### G. SOFTWARE

The operations described above to generate collections of polypeptide sequences can be performed with the assistance of one or more computer programs (software) executing on a computer. The following description of a suitable computer system and software is an exemplary, for purposes of illustration only. Other suitable computer systems and software can be used by one of skill in the art to perform the methods.

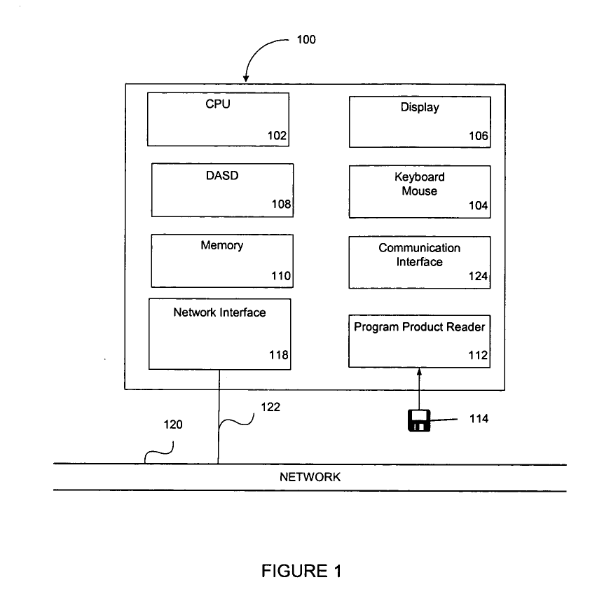

FIG. 1 is an example of a suitable computer system 100 that can implement the functionality described herein. FIG. 1 shows an exemplary computer 100 such as might comprise a conventional desktop computer or workstation. Each computer 100 operates under control of a central processor unit (CPU) 102, such as a “Pentium 4” microprocessor and associated integrated circuit chips, available from Intel Corporation of Santa Clara, Calif., USA. A computer user can input commands and data from a keyboard and computer mouse 104, and can view inputs and computer output at a display 106. The display is typically a video monitor or flat panel display. The computer 100 also includes a direct access storage device (DASD) 108, such as a hard disk drive. The memory 110 typically comprises volatile semiconductor random access memory (RAM). Each computer preferably includes a program product reader 112 that accepts a program product storage device 114, from which the program product reader can read data (and to which it can optionally write data). The program product reader can comprise, for example, a disk drive, and the program product storage device can comprise corresponding removable storage media such as a magnetic floppy disk, a CD-R disc, a CD-RW disc, or DVD-format disc.

Each computer 100 can communicate with other computers over a computer network 120 (such as the Internet or an intranet) through a network interface 118 that enables communication over a connection 122 between the network 120 and the computer. The network interface 118 typically comprises, for example, a Network Interface Card (NIC) and a modem that permits communications over a variety of networks. The computer 100 also can communicate with other devices or computers through a communication interface 124. The communication interface can comprise, for example, a USB connector or a “FireWire” (IEEE 1394) connector.

The CPU 102 operates under control of programming steps that are temporarily stored in the memory 110 of the computer 100. When the programming steps are executed, the computer performs its functions. Thus, the programming steps implement the functionality of the computer. The programming steps can be received from the DASD 108, through the program product storage device 114, or through the network connection 122. The program product storage drive 112 can receive a program product 114, read programming steps recorded thereon, and transfer the programming steps into the memory 110 for execution by the CPU 102. As noted above, the program product storage device can comprise any one of multiple removable media having recorded computer-readable instructions, including magnetic floppy disks and CD-ROM storage discs. Other suitable program product storage devices can include magnetic tape and semiconductor memory chips. In this way, the processing steps necessary for operation in accordance with the invention can be embodied on a program product.

Alternatively, the program steps can be received into the operating memory 110 over the network 120. In the network method, the computer receives data including program steps into the memory 110 through the network interface 118 after network communication has been established over the network connection 122 by well-known methods that will be understood by those skilled in the art without further explanation. The program steps are then executed by the CPU 102 thereby comprising a computer process.

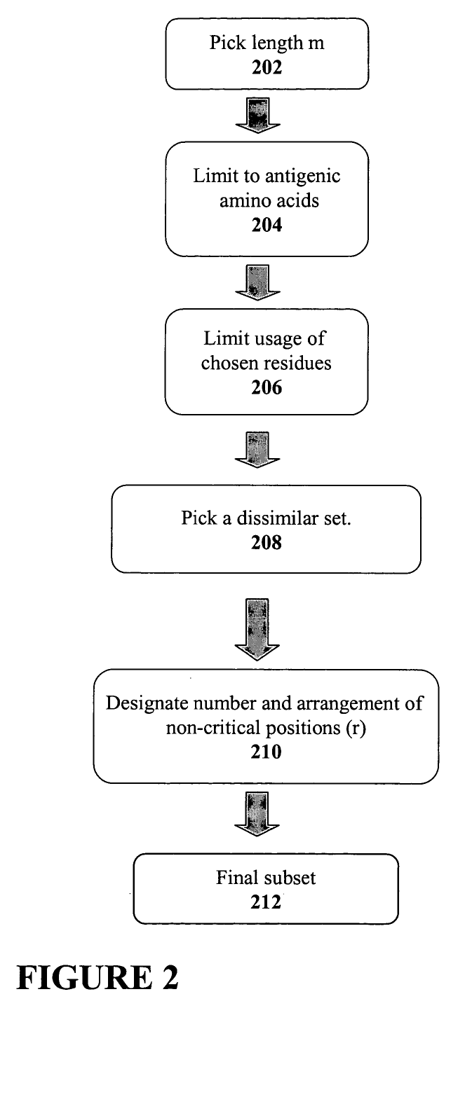

FIG. 2 is a flow diagram of one embodiment of operations that are performed with the computer system of FIG. 1 to generate collections of polypeptide sequences as described above. The operations can be performed as a result of executing one or more computer programs, referred to as software, whose functionality implements the features described above. In the first operation, indicated by the flow diagram box numbered 202 in FIG. 2, a length “m” is selected for a set of polypeptides. The length “m” can be selected as described herein and can be provided as input to the computer system by an operator.

In the next operation, the list of possible amino acids from which the polypeptides will be generated is limited using a ranking of amino acids where n amino acids are ranked. In one example, amino acids are ranked according to their antigenicity such as described herein. The list (subset B) can be generated by a computer program operation that generates an initial list of polypeptides (limited to length “m” by step 202) using a list of ranked amino acids such as a list of antigenically ranked amino acids, such as can be maintained in a computer database or can be located in a data library accessed by the software. This operation is represented by the flow diagram box numbered 204. The Subset B generated by the operation 204 can be significantly smaller than the starting list of possible polypeptides generated if all possible amino acids were used or the top “x”. For example, a 4-mer can have as many as 160,000 possible polypeptides for evaluation if all 20 naturally-occurring amino acids are used, but after the operation of box 202 and 204, the Subset B for a 4-mer can have about 10,000 polypeptides, for example, if 10 amino acids from a ranked list are used.

The next operation is to optionally limit the usage of the chosen residues, represented by the flow diagram box numbered 206. For example usage of the amino acids within the polypeptides can be limited such that there are no multiples of any given amino acid and each amino acid within a given polypeptide sequence is unique. This operation results in a subset C of the set of possible polypeptides. For example, in a 4-mer created with 10 amino acids from a ranked list, no multiples of an amino acid are permitted within a given polypeptide, 5040 polypeptide sequences can be generated in subset C. In one embodiment, operations represented by the boxes numbered 204 and 206 are combined into a single operation.

The next operation, represented by the box numbered 208, selects a subset C of polypeptides from subset C which have similarity values below a selected value. A similarity matrix, such as described herein is used to generate similarity values for all the polypeptides within subset C. Similarity matrices such as described herein, can be maintained for example, in a computer database or can be located in a data library accessed by the software. To generate similarity values for each polypeptide in subset C, a single polypeptide must be chosen from subset C to use as a reference. Such reference polypeptide can be chosen at random or by designating a particular position within the list of subset C polypeptides as the reference.

The next operation, represented by the box numbered 210, selects a number of non-critical amino acids, r, and selects the positions within the subset D polypeptides at which the non-critical r positions will be inserted, such that a pattern of m and r residues is selected and the non-critical amino acids are inserted into the polypeptides of subset D in the selected pattern. Optionally, more than one pattern of m and r residues can be selected, such that for each polypeptide in subset C, a number of polypeptides containing non-critical residues are generated, differing in the arrangement of the critical and non-critical amino acid positions. A list of amino acids designated for non-critical amino acid positions can be used to select amino acids at r positions. The final operation, represented by the box numbered 212 generates a list of polypeptides representing the final subset E.

The operational process illustrated by the flow diagram of FIG. 2 can be performed on the computer system illustrated in FIG. 1 by using one or more computer program to run different software routines. It should be understood that all routines can be integrated into a single computer program or can be performed by multiple programs with and arrangement of program steps. Programs to be employed rely on a suitable database of amino acid data, such as antigenicity and similarity rankings, from which amino acids are selected and from which amino acids and polypeptide sequences are compared. Such databases are readily available and those skilled in the art will be knowledgeable with regard to extracting the appropriate data (see for example, Geysen et al., (1988). J. Molecular Recognition 1:32-41).

### H. DIAGNOSTICS

The methods provided herein generate collections of HAHS polypeptides which can be utilized as a diverse collection of epitopes for diagnostic assays, such as diagnostics for diseases and conditions. For example, a collection of HAHS polypeptides is generated and used to assess the antibodies present in a sample, such as from an animal, subject or patient. Collection of HAHS polypeptides are generated in an addressable format, such as arrayed such on a solid support or associated with color-coded or tagged beads. The addressable collection of HAHS polypeptides is then contacted with samples containing antibodies. Samples can include any fluids, tissues and/or cells which contain antibodies and/or fragments of antibodies, such as but not limited to, blood, sera, spleen, lymph tissue, bone marrow, lymphocytes, plasma cells and B cells. Diagnostic assays can include assessing the number or pattern of HAHS polypeptides bounds and/or the amount of each HAHS polypeptide bound. Results can be compared between samples, or between a sample and a control. For example, a sample from a diseased subject can be compared with a control non-diseased sample. Subjects can be treated with an agent, such as a small molecule, a pathogen and or one or more antigens, samples collected and tested against the collection of HAHS polypeptides. Such samples can be compared with untreated controls to assess differences in antibody levels or types between treated and untreated samples.

Diagnostic assays also can include the use of capture agents with HAHS polypeptides. For example, competitive and displacement assays can be designed using pairs of HAHS polypeptides and capture agents which bind to them. Such pairs can be displayed in an addressable format and a sample then added. In some cases, labeled or otherwise detectable capture agents can be used. Antibodies in the sample compete or displace the capture agents and the amount and/or pattern of competition/displacement can be assessed between samples or between a sample and a control.

### I. EXAMPLES

The following examples are included for illustrative purposes only and are not intended to limit the scope of the invention.

### Example 1

### Generation of a Set of Polypeptide Binding Partner Sequences

The methods provided herein start with a set of amino acids, which typically includes some or all of the naturally-occurring amino acids and also can include selected non-naturally occurring amino acids. For exemplification, the naturally occurring 20 amino acids were included. The polypeptide that is to be designed can be any length, typically is short, at least two amino acids up to 50, but generally is 4, 5, 6, 7, 8, 9, 10, 12, 16, 20 or more. Two amino acids can be sufficient for antigenicity (Geysen et al. (1985) Immunology Today 6(12): 364-369). For exemplification, a length “q” of 6 amino acids was chosen, containing 4 critical residues (m=4). The exemplary initial analysis was performed for 4-mers that contain any of the 20 naturally-occurring amino acids. Accordingly, the total possible set was 20⁴ combinations (mⁿ combinations where m is the number of critical residues and n is the number of amino acid possibilities at these positions).

Further selections were made to generate subsets of polypeptides; members of the subsets were selected by imposing criteria based upon empirical data regarding antigenicity in a particular host and also upon properties of particular amino acids. The targeted host for antigenicity chosen for exemplification was mice.

Step 1: A length of polypeptide q and critical residue number m were chosen. For exemplification a length of 6 was selected with 4 critical residues.

Step 2: A subset was generated with all combinations of 4 residues using 10 amino acids such that there were no duplications of amino acids in any polypeptide(where y=10, the number of chosen amino acids for use in critical positions). The ten amino acids were selected based upon antigenicity ranking (see Table 2). The ranking of amino acids was empirically determined and based on the occurrence of the amino acids in antigenic polypeptides (Geysen et al., (1988). J. Molecular Recognition 1:32-41). The resulting subset contained 5040 members (members total=y!/(y−m)!=10×9×8×7).

Step 3: A further subset was selected containing a dissimilar collection of polypeptide. To start, one polypeptide was chosen from the subset of step 2 (this choice was made by choosing one polypeptide of the preceding subset at random). Using the selected polypeptide and a similarity table (for example, Table 3 was used), a subset of predetermined number of members was chosen. These polypeptide members were selected to contain a sequence of amino acids that is as dissimilar as possible from the other members in the final selected set. This selection was done using the similarity table to create an indexing number, a similarity score, representative of the dissimilarity. A similarity score was obtained by combining the numbers from the table for each amino acid in a particular polypeptide compared to the reference polypeptide to create a score for each of the polypeptides and then selecting a predetermined number by setting a threshold similarity index.

Step 4: Since 4 residues ere selected from the total selected length of 6 (step 3), the remaining 2 residues, designated “non-critical” were then assigned. For exemplary purposes, the 2 non-critical residues were assigned adjacent positions and only critical residues were chosen to occupy the N-terminal and C-terminal positions, thereby generating the possible 6-mers into which non-critical residues were placed. For exemplification, two possible combinations of non-critical residues were selected. These were Tyr-Gly, and Ser-Gly. These were chosen because they confer improved solubility and permit hairpin folding which can be advantageous for generating capture agents/binding partners for the methods and products herein.

The final exemplary set chosen is provided herein (see SEQ ID NOs; 1-911). As shown in Example 2, tested polypeptides resulted in antibodies useful as capture agents specific for the 6-mer polypeptides. Thus, this method permits design of polypeptides that predictably induce production of specific antibodies upon administration, thereby providing highly specific capture agent/binding partner pairs for use in the methods and products provided herein.

### Example 2

### Generation of Binding Partner-Capture Agent Pairs

A. Generation of 6-mer Polypeptide Epitope Tags

A collection of 6 amino acid polypeptides (6-mers) were designed using the method described herein for designing highly antigenic, highly specific peptides. The polypeptides were designed for screening suitability and use as binding partners paired with capture agents.

Peptides (6-mers) were synthesized with a C-terminal cysteine residue as: cysteine-(amino acid)₆-NH₂. Diphtheria toxoid was activated using MCS to add maleimido groups to lysine side chains (Lee et al. (1985) Mol. Immunol. 17:749-756). A 1.5 molar excess of the activated carrier protein was incubated with the polypeptides. The ratio ensures the lack of free unconjugated polypeptides such that unconjugated polypeptides or carrier proteins are not separated from the conjugated sample. The 6-mer polypeptides also are synthesized with biotin at the C-terminal end with a 4-mer linker polypeptide for use in screening assays: Biotin-SGSG-(amino acid)6-NH₂.

B. Immunization of Mice with DT-Peptide Conjugates

The DT-peptide conjugates were dissolved in PBS. To formulate the mixture of conjugates, 0.5 mg of each of four peptides is added into one tube and the volume made to 2 ml with sterile PBS. The conjugates are mixed well before dispensing so that any particulate is well suspended. Each group of four polypeptide conjugates is designated by a group name, for example, as Grp1, Grp2, Grp3, and so on.

Three mice were immunized with each group of polypeptide conjugates. Mice were immunized with 200 μg protein/mouse for initial immunization (day 0) and boosts of 100 μg protein/mouse at days 21, 35, 49 and 63. Tail bleeds were taken at day 42 and day 70 and analyzed by ELISA assays. Samples of serum were taken from tail bleeds of the mice before day 0 immunizations to serve as pre-immune control serum.

Mice were analyzed by ELISA as follows. Biotinylated polypeptides were dissolved in DMSO at final concentrations of 5 mg/ml. NUNC Maxisorp plates are coated with 5 μg/ml Neutravidin in PBS and incubated at 4° C. until use (up to 30 days). The NeutrAvidin is aspirated off and the plates incubated with biotinylated polypeptides at 5μg/ml in PBS for 60 min at 37° C. as indicated in the table below.

| | Plate 1 | Plate 2 | Plate 3 | Plate 4 | Plate 5 | Plate 6 |
|---|---|---|---|---|---|---|
| A | Peptide 1 | Peptide 9 | Peptide 17 | Peptide 25 | Peptide 33 | Peptide 41 |
| B | Peptide 2 | Peptide 10 | Peptide 18 | Peptide 26 | Peptide 34 | Peptide 42 |
| C | Peptide 3 | Peptide 11 | Peptide 19 | Peptide 27 | Peptide 35 | Peptide 43 |
| D | Peptide 4 | Peptide 12 | Peptide 20 | Peptide 28 | Peptide 36 | Peptide 44 |
| E | Peptide 5 | Peptide 13 | Peptide 21 | Peptide 29 | Peptide 37 | Peptide 45 |
| F | Peptide 6 | Peptide 14 | Peptide 22 | Peptide 30 | Peptide 38 | Peptide 46 |
| G | Peptide 7 | Peptide 15 | Peptide 23 | Peptide 31 | Peptide 39 | Peptide 47 |
| H | Peptide 8 | Peptide 16 | Peptide 24 | Peptide 32 | Peptide 40 | Peptide 48 |

The plates were blocked with 1X Blocker BSA in PBS-T for 60 min at 37° C. One hundred microliters of each tail-bleed sample is added to Row A at a 1:100 dilution (2.5 μl of a 1:10 diluted tail-bleed and 22.5μl Blocker BSA). To each plate, tail bleeds were added as follows (group refers to the groups of polypeptide-conjugates used for immunization, Mu1-Mu9 refer to the individual mice that were immunized with each group of peptides, described above).

| 1 | 2 | 3 | 4 | 5 | 6 | 7 | 8 | 9 |
|---|---|---|---|---|---|---|---|---|
| Tail | Tail | Tail | Tail | Tail | Tail | Tail | Tail | Tail |
| bleed | bleed | bleed | bleed | bleed | bleed | bleed | bleed | bleed |
| Grp1 | Grp1 | Grp1 | Grp2 | Grp2 | Grp2 | Grp3 | Grp3 | Grp3 |
| Mu1 | Mu2 | Mu3 | Mu4 | Mu5 | Mu6 | Mu7 | Mu8 | Mu9 |

The plates were incubated for 60 min at 37° C. and then washed 3X with 1X TBS-T. They then were incubated with 100 μl of a 1:2000 dilution of goat anti-mouse IgG-HRP conjugate for 60 min at 37° C., washed again 3 times with TBS-T and developed with OPD. The absorbance measured at 492 nm.

C. Generation of a Library of Hybridoma Cells

An additional 1.2 mg of conjugate-peptide mixtures (0.3 mg of each) was prepared for injection into mice prior to fusion. The mice were boosted with injections of polypeptides for three days prior to fusion. Fusion of spleen cells with mouse myeloma cells was performed on Day 84 and the hybridoma cells were grown in selection medium for 4 weeks. The medium was removed 3 weeks after fusion and fresh medium was added. The medium was harvested on Week 4 after fusion and tested for presence of anti-peptide antibodies by ELISA as described above. The assay was performed only for determination of antibodies to the immunized polypeptides and not for cross-reactivity. The cells were harvested, aliquoted and stored (Fusion library) until the results from analysis of supernatants were obtained.

D. Cloning of Hybridomas to Generate Monoclonal Antibodies

A vial of the fusion library was thawed and the cells grown in medium for 2 weeks. Cells then were sorted using a FACS into ten 96-well plates such that each well received a single cell. The cells were grown for 2 weeks and the supernatant from each clone analyzed for presence of anti-peptide antibody as for the fusion library supernatant.

Positive clones were identified and ranked in order of ELISA signal intensities. Twelve clones with the highest signal intensities were scaled-up and assayed for polypeptide-specific antibody after 2 weeks. The supernatants then were assayed for antibody titre determination and two clones showing the highest anti-peptide antibody titre were selected for scale-up and storage. The clones were grown to obtain 100 ml of medium and the cells then were frozen at −80° C.

E. Purification and Isotyping of IgG from Hybridoma Lines

The selected clones were grown for 2 weeks and the medium was used for analysis of antibody class and for specificity of binding to polypeptides by performing the assay described above. IgG was isotyped using Isotype mouse isotyping kits (Roche). The antibody from the supernatant was purified using Protein G affinity chromatography and stored in liquid nitrogen.

F. Results

Peptides used for the immunizations were as follows:

| | | | |
|---|---|---|---|
| SEQ ID NO: | Peptide | SEQ ID NO: | Peptide |

| | | | |
|---|---|---|---|
| 1 | EPNGYF | 287 | QGKEYF |
| 5 | EGYPNF | 344 | NSFEGP |
| 137 | PEQGYN | 346 | NFKSGH |
| 141 | PGYEQN | 350 | NSGFKH |
| 236 | QESGPD | 351 | NGFKYH |
| 251 | QPGYEH | 372 | NTSGHK |
| 329 | NQHGYD | 379 | NKGYHL |
| 341 | NGYFEP | 428 | FPSGNE |
| 8 | ESPNGF | 450 | FNPSGE |
| 10 | EPHSGK | 454 | FSGNPE |
| 14 | ESGPHK | 455 | FGNPYE |
| 15 | EGPHYK | 481 | FTLGYQ |
| 19 | EQGYPN | 485 | FGYTLQ |
| 28 | EQSGFH | 488 | FSTLGQ |
| 144 | PSEQGN | 566 | HSGQEL |
| 146 | PEFSGQ | 570 | HQTSGN |
| 150 | PSGEFQ | 585 | HNDGYT |
| 151 | PGEFYQ | 595 | HFGYTK |
| 155 | PEGYKD | 636 | HDSGTL |
| 172 | PNSGEF | 691 | TLGYNF |
| 261 | OGYNHE | 735 | KGQNYT |
| 264 | QSNHGE | 747 | KNGYDQ |
| 265 | QEEGYK | 773 | KGYHPD |
| 282 | QKESGF | 776 | KSHPGD |

Peptides were injected singly or in groups of 2-4 polypeptides/animal as described above. Antisera were analyzed as described. The injected polypeptides raised antisera with high specificity and affinity.

Since modifications will be apparent to those of skill in this art, it is intended that this invention be limited only by the scope of the appended claims.

1. A collection of antigenic polypeptides, comprising:
at least three antigenic polypeptides that comprise 5 to 8 unique residues and include at least 4 residues, designated critical residues, selected from E, P, Q, N, F, H, T, K, L, D, wherein:
critical residues occupy the N and C terminal positions in each polypeptide; and no more than three polypeptides in the collection contain the same four critical residues.

2. The collection of claim 1, wherein at least two polypeptides in the collection comprise the same four critical residues but each of the two polypeptides has non-critical residues at different positions; and wherein the polypeptides comprise at least 6 unique residues and at least 2 non-critical residues that are adjacent to each other.

3. The collection of claim 2, wherein the non-critical residues are selected from among Y, S and G.

4. The collection of claim 1, wherein the polypeptides comprise 6 unique residues.

5. The collection of claim 1, wherein the polypeptides comprise 7 unique residues.

6. The collection of claim 1 comprising at least 4, 5, 6, 7, 8, 9, 10, 20, 25, 30, 40, 50, 60, 70, 80, 90 or 100 members.

7. The collection of claim 1 comprising at least 200, 300, 400, 500, 750 or 1000 members.

8. The collection of claim 1 comprising at least 5000 or 10,000 members.

9. The collection of claim 1, wherein the polypeptides are at least 6, 7, 8, 9, 10, 11, 12, 15, 20, 25, 30, 35, 40, or 45 amino acids in length.

10. The collection of claim 1, wherein the polypeptides are between 6 and 8 or 6 and 10 or 6 and 12 or 6 and 15 or 6 and 20 amino acids in length.

11. The collection of claim 1, wherein the polypeptides are antigenic in a non-human subject.

12. The collection of claim 11, wherein the polypeptides exhibit higher antigenicity in a non-human subject than in a human subject.

13. The collection of claim 12, wherein the non-human subject is a rodent or a bird.

14. The collection of claim 1 that is addressable.

15. The collection of claim 1, wherein the polypeptides of the collection are positionally addressable.

16. The collection of claim 1, wherein the polypeptides of the collection are immobilized on a solid support.

17. The collection of claim 1, wherein the polypeptides are conjugated to members of a library.

18. The collection of claim 17, wherein the library is selected from a nucleic acid library, a polypeptide library, a natural products library, and a combinatorial chemistry library.

19. The collection of claim 1, wherein the polypeptides of the collection are conjugated to molecules selected from the group consisting of polypeptides, nucleic acids and small organic molecules.

20. A collection of capture agent—binding partner polypeptide pairs comprising:
a collection of binding partners comprising a collection of antigenic polypeptides according to claim 1; and
a collection of capture agents, wherein the capture agents each bind to a unique binding partner within the collection of antigenic polypeptides.

21. The collection of claim 20, wherein the capture agents comprise antibodies or antibody fragments.

22. The collection of claim 20, wherein the collection of binding partners include 2, 3, 4, 5, 6, 7, 8, 9, 10, 15, 20, 50, 100 or more polypeptides of any of SEQ ID Nos. 1-911.

23. A collection of binding partner polypeptides, comprising 2, 3, 4, 5, 6, 7, 8, 9, 10, 15, 20, 50, 100 or more polypeptides of any of SEQ ID Nos. 1-911.

24. A fusion protein, comprising a first polypeptide or one or more amino acids conjugated to any of the polypeptides set forth as SEQ ID Nos. 1-911.

25. A fusion protein of claim 24 that is at least 6, 7, 8, 9, 10, 11, 12, 15, 20, 25, 30, 35, 40 or 45 amino acids in length.

26. A method of generating highly antigenic highly specific binding polypeptides, comprising:
a) ranking amino acids based upon pre-determined criteria for antigenicity, wherein n amino acids are ranked;
b) based upon the ranking using the top m to n-1 ranked amino acids, generating all combinations of the amino acids in a polypeptide of pre-selected length m residues to produce a set S1 of polypeptides of length m residues, wherein:
n is the number of amino acids in a preselected set of possible amino acids; and
m is a length of amino acids preselected to have a minimum length to result in sufficient affinity to bind to a selected capture agent up to a length that retains specific binding to a selected capture agent; and
c) based upon pre-determined criteria for dissimilarity, selecting a subset of set of dissimilar polypeptides from set S1.

27. The method of claim 26, wherein the pre-determined criteria for antigenicity is based upon frequency of the amino acids in a pre-selected set of antigenic polypeptides.

28. The method of claim 26, wherein n is equal to 5, 6, 7, 8, 9, 10, 11, 12, 13, 14, 15, 16, 17, 18 or 19.

29. The method of claim 26, wherein m is equal to 4, 5, 6, 7, 8, 9, 10, 11, 12, 13, 14, 15, 16, 17, 18, 19, 20, 21, 22, 23, 24, 25, 26, 27, 28, 29 or 30.

30. The method of claim 26, wherein m is an integer between and including 4 and 6 or 4 and 7 or 4 and 8 or 4 and 12 or 5 and 7 or 5 and 8 or 5 and 12 or 6 and 8 or 6 and 10 or 6 and 12 or 8 and 12.

31. The method of claim 26, wherein the amino acids are selected from among E, P, Q, N, F, H, T, K, L, D, S, G and Y.

32. The method of claim 26, wherein the amino acids are naturally-occurring amino acids.

33. The method of claim 26, wherein the amino acids include non-naturally occurring amino acids.

34. The method of claim 33, wherein the amino acids include non-naturally occurring and naturally-occurring amino acids.

35. The method of claim 34, wherein n is between 20 and 10,000.

36. The method of claim 26, further comprising generating a subset of polypeptides of length q residues, wherein q=m+r; r is the number of non-critical amino acids; r is an integer equal to or greater than 1; and q is an integer greater than 4.

37. The method of claim 36, wherein the N and C terminal amino acids of the polypeptides of length q residues are critical amino acids.

38. The method of claim 37, wherein at least 2 of the non-critical amino acids are adjacent.

39. The method of claim 36, wherein r is 1, 2, 3, 4, 5, 6, 7, 8, 9 or 10.

40. The method of claim 39, wherein r is 2, 3 or 4.

41. The method of claim 36, wherein q is an integer between 5 and 100 or 5 and 50 or 5 and 30 or 5 and 20 or 5 and 10.

42. The method of claim 26, wherein:
dissimilarity is assessed by comparing each polypeptide in the set S1 to an arbitrarily selected reference polypeptide from the set S1 by comparing corresponding critical residues based upon position in the polypeptides; and wherein polypeptides from set S1 are selected that contain corresponding critical residues most dissimilar from the reference polypeptide; and dissimilarity refers to functional and structural dissimilarity based upon predetermined criteria.

43. The method of claim 42, wherein dissimilarity is determined by calculating a similarity score from a similarity matrix by comparing values for the corresponding critical residues in the reference polypeptide to the corresponding critical residues in polypeptides of set S1; combining the scores for the residues in each polypeptide to generate a score for each polypeptide; and selecting those below a predetermined score.

44. A collection of binding partner polypeptides, comprising 2, 3, 4, 5, 6, 7, 8, 9, 10, 15, 20, 50, 100 or more polypeptides generated by the method of claim 26.

45. A collection of capture agent—binding partner polypeptide pairs, comprising the collection of binding partner polypeptides of claim 44 and a collection of capture agents, wherein the capture agents each bind to a binding partner polypeptide within the collection of binding partner polypeptides.

46. A kit, comprising:
a collection of claim 44; and optionally including instructions preparing capture agents that specifically bind to members of the collection.

47. A method for synthesizing an addressable collection of polypeptides comprising:
a) providing a collection of b tags and a collection of b addressable capture agents, wherein each capture agent binds a unique tag and b is the number of tag-capture agent pairs;
b) presenting the tags in an addressable format suitable for peptide synthesis;
c) synthesizing a collection of polypeptides on the collection of tags, wherein each tag is conjugated directly or indirectly via a linker to a synthesized polypeptide, wherein each synthesized polypeptide comprises a number of variable amino acid positions v and optionally a number n of fixed amino acid positions each designated N;
each N can be the same or different; and
the method of synthesis comprises:
i) synthesizing a subset of v positions in a first round of synthesis to generate a collection of tag-v1 polypeptides, whereby each unique tag is directly or indirectly linked to an amino acid, wherein each tag has a unique combination of amino acids at the synthesized variable positions;
ii) mixing the collection of synthesized tag-v1 polypeptides and splitting the collection of tag-v1 polypeptides into b addressable first-round subsets, whereby each first-round subset contains a collection of tag-v1 polypeptides representing on average every possible combination of amino acids at the synthesized variable positions;
iii) synthesizing a further subset of variable positions v2 in a further round of synthesis, such that each tag-v1 polypeptide is conjugated to a unique combination of amino acids at v2 positions to generate b subsets of tag-v1v2 polypeptides;
iv) contacting the resulting subsets of tag-v1v2 polypeptides with the addressable collection of b capture agents to produce an addressable collection of synthesized polypeptides.

48. The method of claim 47, wherein v is equal to 4 and the subset of variable positions synthesized in the first round is equal to 2.

49. The method of claim 47, further comprising:
d) incubating the addressable collection of synthesized polypeptides or a subset thereof with one or more collections of molecules under conditions where one or more molecules specifically binds to the synthesized polypeptides.

50. The method of claim 49, wherein the collections of molecules comprise molecules selected from the group consisting of antibodies, fragments of antibodies and polypeptides.

51. The method of claim 47, wherein the collection of b capture agents are conjugated to a solid support.

52. The method of claim 51, wherein b collections of b capture agents are conjugated to a solid support.

53. The method of claim 47, wherein the capture agents comprise antibodies or antibody fragments.

54. The method of claim 47, wherein the synthesized polypeptides are of length d, wherein d=n+v and n is an integer equal to or greater than 1.

55. The method of claim 54, wherein n is 2, 3, 4, 5, 6, 7, 8, 9, 10, 11, or 12.

56. The method of claim 47, wherein v is 4, 5, 6, 7 or 8.

57. The method of claim 47, wherein the synthesized polypeptides are highly antigenichighly specific polypeptides.

58. A method for synthesizing an addressable collection of molecules comprising:
a) providing a collection of b tags and a collection of b addressable capture agents, wherein each capture agent binds a unique tag and b is the number of tag-capture agent pairs;
b) presenting the tags in an addressable format suitable for chemical synthesis;
c) synthesizing a collection of molecules, wherein the molecules are synthesized on starting molecules;
each tag is conjugated directly or indirectly via a linker to a starting molecule;
each synthesized molecule comprises a number of variable constituent positions X conjugated to the starting molecule;
and the method of synthesis comprises:
i) synthesizing a subset of X positions X1 in a first round of synthesis to generate a collection of tag-X1 molecules, whereby each unique tag is conjugated to a unique combination of constituents at the synthesized X1 positions;
ii) mixing the collection of synthesized tag-X1 molecules and splitting the collection of tag-X1 molecules into b addressable first-round subsets, whereby each first-round subset contains a collection of tag-X1 molecules representing on average every possible combination of constituents at the synthesized X1 positions;
iii) synthesizing a further subset of constituent positions X2 in a further round of synthesis, such that each first-round subset is conjugated to a unique combination of constituents at X2 positions to generate b second-round subsets; and
iv) contacting the resulting tag-X1X2 with an addressable collection of b capture agents to produce an addressable collection of synthesized molecules.

59. The method of claim 58, wherein the synthesized molecules are selected from the group consisting of nucleic acid molecules, polymers, biopolymers, polypeptides, and small organic molecules.

60. The method of claim 58, wherein the starting molecule is a pharmacophore.

61. The method of claim 58, wherein the starting molecule is a monomer and the synthesized molecules are polymers.

62. The method of claim 58, wherein the tags are highly antigenic, highly specific polypeptides.

63. The method of claim 58, wherein the capture agents comprise antibodies or antibody fragments.

64. The method of claim 58, wherein the tags comprise any of the polypeptides set forth in SEQ ID Nos. 1-911.


SEQUENCE LISTING (911 sequences)

SEQ ID NO: 1 - protein, 6 aa, Artificial Sequence
  synthetic peptide
EPNGYF

SEQ ID NO: 2 - protein, 6 aa, Artificial Sequence
  synthetic peptide
EPNSGF

SEQ ID NO: 3 - protein, 6 aa, Artificial Sequence
  synthetic peptide
EPGYNF

SEQ ID NO: 4 - protein, 6 aa, Artificial Sequence
  synthetic peptide
EPSGNF

SEQ ID NO: 5 - protein, 6 aa, Artificial Sequence
  synthetic peptide
EGYPNF

SEQ ID NO: 6 - protein, 6 aa, Artificial Sequence
  synthetic peptide
ESGPNF

SEQ ID NO: 7 - protein, 6 aa, Artificial Sequence
  synthetic peptide
EGPNYF

SEQ ID NO: 8 - protein, 6 aa, Artificial Sequence
  synthetic peptide
ESPNGF

SEQ ID NO: 9 - protein, 6 aa, Artificial Sequence
  synthetic peptide
EPHGYK

SEQ ID NO: 10 - protein, 6 aa, Artificial Sequence
  synthetic peptide
EPHSGK

SEQ ID NO: 11 - protein, 6 aa, Artificial Sequence
  synthetic peptide
EPGYHK

SEQ ID NO: 12 - protein, 6 aa, Artificial Sequence
  synthetic peptide
EPSGHK

SEQ ID NO: 13 - protein, 6 aa, Artificial Sequence
  synthetic peptide
EGYPHK

SEQ ID NO: 14 - protein, 6 aa, Artificial Sequence
  synthetic peptide
ESGPHK

SEQ ID NO: 15 - protein, 6 aa, Artificial Sequence
  synthetic peptide
EGPHYK

SEQ ID NO: 16 - protein, 6 aa, Artificial Sequence
  synthetic peptide
ESPHGK

SEQ ID NO: 17 - protein, 6 aa, Artificial Sequence
  synthetic peptide
EQPGYN

SEQ ID NO: 18 - protein, 6 aa, Artificial Sequence
  synthetic peptide
EQPSGN

SEQ ID NO: 19 - protein, 6 aa, Artificial Sequence
  synthetic peptide
EQGYPN

SEQ ID NO: 20 - protein, 6 aa, Artificial Sequence
  synthetic peptide
EQSGPN

SEQ ID NO: 21 - protein, 6 aa, Artificial Sequence
  synthetic peptide
EGYQPN

SEQ ID NO: 22 - protein, 6 aa, Artificial Sequence
  synthetic peptide
ESGQPN

SEQ ID NO: 23 - protein, 6 aa, Artificial Sequence
  synthetic peptide
EGQPYN

SEQ ID NO: 24 - protein, 6 aa, Artificial Sequence
  synthetic peptide
ESQPGN

SEQ ID NO: 25 - protein, 6 aa, Artificial Sequence
  synthetic peptide
EQFGYH

SEQ ID NO: 26 - protein, 6 aa, Artificial Sequence
  synthetic peptide
EQFSGH

SEQ ID NO: 27 - protein, 6 aa, Artificial Sequence
  synthetic peptide
EQGYFH

SEQ ID NO: 28 - protein, 6 aa, Artificial Sequence
  synthetic peptide
EQSGFH

SEQ ID NO: 29 - protein, 6 aa, Artificial Sequence
  synthetic peptide
EGYQFH

SEQ ID NO: 30 - protein, 6 aa, Artificial Sequence
  synthetic peptide
ESGQFH

SEQ ID NO: 31 - protein, 6 aa, Artificial Sequence
  synthetic peptide
EGQFYH

SEQ ID NO: 32 - protein, 6 aa, Artificial Sequence
  synthetic peptide
ESQFGH

SEQ ID NO: 33 - protein, 6 aa, Artificial Sequence
  synthetic peptide
ENPGYT

SEQ ID NO: 34 - protein, 6 aa, Artificial Sequence
  synthetic peptide
ENPSGT

SEQ ID NO: 35 - protein, 6 aa, Artificial Sequence
  synthetic peptide
ENGYPT

SEQ ID NO: 36 - protein, 6 aa, Artificial Sequence
  synthetic peptide
ENSGPT

SEQ ID NO: 37 - protein, 6 aa, Artificial Sequence
  synthetic peptide
EGYNPT

SEQ ID NO: 38 - protein, 6 aa, Artificial Sequence
  synthetic peptide
ESGNPT

SEQ ID NO: 39 - protein, 6 aa, Artificial Sequence
  synthetic peptide
EGNPYT

SEQ ID NO: 40 - protein, 6 aa, Artificial Sequence
  synthetic peptide
ESNPGT

SEQ ID NO: 41 - protein, 6 aa, Artificial Sequence
  synthetic peptide
ENFGYD

SEQ ID NO: 42 - protein, 6 aa, Artificial Sequence
  synthetic peptide
ENFSGD

SEQ ID NO: 43 - protein, 6 aa, Artificial Sequence
  synthetic peptide
ENGYFD

SEQ ID NO: 44 - protein, 6 aa, Artificial Sequence
  synthetic peptide
ENSGFD

SEQ ID NO: 45 - protein, 6 aa, Artificial Sequence
  synthetic peptide
EGYNFD

SEQ ID NO: 46 - protein, 6 aa, Artificial Sequence
  synthetic peptide
ESGNFD

SEQ ID NO: 47 - protein, 6 aa, Artificial Sequence
  synthetic peptide
EGNFYD

SEQ ID NO: 48 - protein, 6 aa, Artificial Sequence
  synthetic peptide
ESNFGD

SEQ ID NO: 49 - protein, 6 aa, Artificial Sequence
  synthetic peptide
ENDGYP

SEQ ID NO: 50 - protein, 6 aa, Artificial Sequence
  synthetic peptide
ENDSGP

SEQ ID NO: 51 - protein, 6 aa, Artificial Sequence
  synthetic peptide
ENGYDP

SEQ ID NO: 52 - protein, 6 aa, Artificial Sequence
  synthetic peptide
ENSGDP

SEQ ID NO: 53 - protein, 6 aa, Artificial Sequence
  synthetic peptide
EGYNDP

SEQ ID NO: 54 - protein, 6 aa, Artificial Sequence
  synthetic peptide
ESGNDP

SEQ ID NO: 55 - protein, 6 aa, Artificial Sequence
  synthetic peptide
EGNDYP

SEQ ID NO: 56 - protein, 6 aa, Artificial Sequence
  synthetic peptide
ESNDGP

SEQ ID NO: 57 - protein, 6 aa, Artificial Sequence
  synthetic peptide
EFQGYP

SEQ ID NO: 58 - protein, 6 aa, Artificial Sequence
  synthetic peptide
EFQSGP

SEQ ID NO: 59 - protein, 6 aa, Artificial Sequence
  synthetic peptide
EFGYQP

SEQ ID NO: 60 - protein, 6 aa, Artificial Sequence
  synthetic peptide
EFSGQP

SEQ ID NO: 61 - protein, 6 aa, Artificial Sequence
  synthetic peptide
EGYFQP

SEQ ID NO: 62 - protein, 6 aa, Artificial Sequence
  synthetic peptide
ESGFQP

SEQ ID NO: 63 - protein, 6 aa, Artificial Sequence
  synthetic peptide
EGFQYP

SEQ ID NO: 64 - protein, 6 aa, Artificial Sequence
  synthetic peptide
ESFQGP

SEQ ID NO: 65 - protein, 6 aa, Artificial Sequence
  synthetic peptide
EFKGYT

SEQ ID NO: 66 - protein, 6 aa, Artificial Sequence
  synthetic peptide
EFKSGT

SEQ ID NO: 67 - protein, 6 aa, Artificial Sequence
  synthetic peptide
EFGYKT

SEQ ID NO: 68 - protein, 6 aa, Artificial Sequence
  synthetic peptide
EFSGKT

SEQ ID NO: 69 - protein, 6 aa, Artificial Sequence
  synthetic peptide
EGYFKT

SEQ ID NO: 70 - protein, 6 aa, Artificial Sequence
  synthetic peptide
ESGFKT

SEQ ID NO: 71 - protein, 6 aa, Artificial Sequence
  synthetic peptide
EGFKYT

SEQ ID NO: 72 - protein, 6 aa, Artificial Sequence
  synthetic peptide
ESFKGT

SEQ ID NO: 73 - protein, 6 aa, Artificial Sequence
  synthetic peptide
EFDGYH

SEQ ID NO: 74 - protein, 6 aa, Artificial Sequence
  synthetic peptide
EFDSGH

SEQ ID NO: 75 - protein, 6 aa, Artificial Sequence
  synthetic peptide
EFGYDH

SEQ ID NO: 76 - protein, 6 aa, Artificial Sequence
  synthetic peptide
EFSGDH

SEQ ID NO: 77 - protein, 6 aa, Artificial Sequence
  synthetic peptide
EGYFDH

SEQ ID NO: 78 - protein, 6 aa, Artificial Sequence
  synthetic peptide
ESGFDH

SEQ ID NO: 79 - protein, 6 aa, Artificial Sequence
  synthetic peptide
EGFDYH

SEQ ID NO: 80 - protein, 6 aa, Artificial Sequence
  synthetic peptide
ESFDGH

SEQ ID NO: 81 - protein, 6 aa, Artificial Sequence
  synthetic peptide
EHNGYQ

SEQ ID NO: 82 - protein, 6 aa, Artificial Sequence
  synthetic peptide
EHNSGQ

SEQ ID NO: 83 - protein, 6 aa, Artificial Sequence
  synthetic peptide
EHGYNQ

SEQ ID NO: 84 - protein, 6 aa, Artificial Sequence
  synthetic peptide
EHSGNQ

SEQ ID NO: 85 - protein, 6 aa, Artificial Sequence
  synthetic peptide
EGYHNQ

SEQ ID NO: 86 - protein, 6 aa, Artificial Sequence
  synthetic peptide
ESGHNQ

SEQ ID NO: 87 - protein, 6 aa, Artificial Sequence
  synthetic peptide
EGHNYQ

SEQ ID NO: 88 - protein, 6 aa, Artificial Sequence
  synthetic peptide
ESHNGQ

SEQ ID NO: 89 - protein, 6 aa, Artificial Sequence
  synthetic peptide
EHKGYP

SEQ ID NO: 90 - protein, 6 aa, Artificial Sequence
  synthetic peptide
EHKSGP

SEQ ID NO: 91 - protein, 6 aa, Artificial Sequence
  synthetic peptide
EHGYKP

SEQ ID NO: 92 - protein, 6 aa, Artificial Sequence
  synthetic peptide
EHSGKP

SEQ ID NO: 93 - protein, 6 aa, Artificial Sequence
  synthetic peptide
EGYHKP

SEQ ID NO: 94 - protein, 6 aa, Artificial Sequence
  synthetic peptide
ESGHKP

SEQ ID NO: 95 - protein, 6 aa, Artificial Sequence
  synthetic peptide
EGHKYP

SEQ ID NO: 96 - protein, 6 aa, Artificial Sequence
  synthetic peptide
ESHKGP

SEQ ID NO: 97 - protein, 6 aa, Artificial Sequence
  synthetic peptide
ETNGYK

SEQ ID NO: 98 - protein, 6 aa, Artificial Sequence
  synthetic peptide
ETNSGK

SEQ ID NO: 99 - protein, 6 aa, Artificial Sequence
  synthetic peptide
ETGYNK

SEQ ID NO: 100 - protein, 6 aa, Artificial Sequence
  synthetic peptide
ETSGNK

SEQ ID NO: 101 - protein, 6 aa, Artificial Sequence
  synthetic peptide
EGYTNK

SEQ ID NO: 102 - protein, 6 aa, Artificial Sequence
  synthetic peptide
ESGTNK

SEQ ID NO: 103 - protein, 6 aa, Artificial Sequence
  synthetic peptide
EGTNYK

SEQ ID NO: 104 - protein, 6 aa, Artificial Sequence
  synthetic peptide
ESTNGK

SEQ ID NO: 105 - protein, 6 aa, Artificial Sequence
  synthetic peptide
EKPGYH

SEQ ID NO: 106 - protein, 6 aa, Artificial Sequence
  synthetic peptide
EKPSGH

SEQ ID NO: 107 - protein, 6 aa, Artificial Sequence
  synthetic peptide
EKGYPH

SEQ ID NO: 108 - protein, 6 aa, Artificial Sequence
  synthetic peptide
EKSGPH

SEQ ID NO: 109 - protein, 6 aa, Artificial Sequence
  synthetic peptide
EGYKPH

SEQ ID NO: 110 - protein, 6 aa, Artificial Sequence
  synthetic peptide
ESGKPH

SEQ ID NO: 111 - protein, 6 aa, Artificial Sequence
  synthetic peptide
EGKPYH

SEQ ID NO: 112 - protein, 6 aa, Artificial Sequence
  synthetic peptide
ESKPGH

SEQ ID NO: 113 - protein, 6 aa, Artificial Sequence
  synthetic peptide
ELNGYD

SEQ ID NO: 114 - protein, 6 aa, Artificial Sequence
  synthetic peptide
ELNSGD

SEQ ID NO: 115 - protein, 6 aa, Artificial Sequence
  synthetic peptide
ELGYND

SEQ ID NO: 116 - protein, 6 aa, Artificial Sequence
  synthetic peptide
ELSGND

SEQ ID NO: 117 - protein, 6 aa, Artificial Sequence
  synthetic peptide
EGYLND

SEQ ID NO: 118 - protein, 6 aa, Artificial Sequence
  synthetic peptide
ESGLND

SEQ ID NO: 119 - protein, 6 aa, Artificial Sequence
  synthetic peptide
EGLNYD

SEQ ID NO: 120 - protein, 6 aa, Artificial Sequence
  synthetic peptide
ESLNGD

SEQ ID NO: 121 - protein, 6 aa, Artificial Sequence
  synthetic peptide
EDPGYF

SEQ ID NO: 122 - protein, 6 aa, Artificial Sequence
  synthetic peptide
EDPSGF

SEQ ID NO: 123 - protein, 6 aa, Artificial Sequence
  synthetic peptide
EDGYPF

SEQ ID NO: 124 - protein, 6 aa, Artificial Sequence
  synthetic peptide
EDSGPF

SEQ ID NO: 125 - protein, 6 aa, Artificial Sequence
  synthetic peptide
EGYDPF

SEQ ID NO: 126 - protein, 6 aa, Artificial Sequence
  synthetic peptide
ESGDPF

SEQ ID NO: 127 - protein, 6 aa, Artificial Sequence
  synthetic peptide
EGDPYF

SEQ ID NO: 128 - protein, 6 aa, Artificial Sequence
  synthetic peptide
ESDPGF

SEQ ID NO: 129 - protein, 6 aa, Artificial Sequence
  synthetic peptide
EDFGYP

SEQ ID NO: 130 - protein, 6 aa, Artificial Sequence
  synthetic peptide
EDFSGP

SEQ ID NO: 131 - protein, 6 aa, Artificial Sequence
  synthetic peptide
EDGYFP

SEQ ID NO: 132 - protein, 6 aa, Artificial Sequence
  synthetic peptide
EDSGFP

SEQ ID NO: 133 - protein, 6 aa, Artificial Sequence
  synthetic peptide
EGYDFP

SEQ ID NO: 134 - protein, 6 aa, Artificial Sequence
  synthetic peptide
ESGDFP

SEQ ID NO: 135 - protein, 6 aa, Artificial Sequence
  synthetic peptide
EGDFYP

SEQ ID NO: 136 - protein, 6 aa, Artificial Sequence
  synthetic peptide
ESDFGP

SEQ ID NO: 137 - protein, 6 aa, Artificial Sequence
  synthetic peptide
PEQGYN

SEQ ID NO: 138 - protein, 6 aa, Artificial Sequence
  synthetic peptide
PEQSGN

SEQ ID NO: 139 - protein, 6 aa, Artificial Sequence
  synthetic peptide
PEGYQN

SEQ ID NO: 140 - protein, 6 aa, Artificial Sequence
  synthetic peptide
PESGQN

SEQ ID NO: 141 - protein, 6 aa, Artificial Sequence
  synthetic peptide
PGYEQN

SEQ ID NO: 142 - protein, 6 aa, Artificial Sequence
  synthetic peptide
PSGEQN

SEQ ID NO: 143 - protein, 6 aa, Artificial Sequence
  synthetic peptide
PGEQYN

SEQ ID NO: 144 - protein, 6 aa, Artificial Sequence
  synthetic peptide
PSEQGN

SEQ ID NO: 145 - protein, 6 aa, Artificial Sequence
  synthetic peptide
PEFGYQ

SEQ ID NO: 146 - protein, 6 aa, Artificial Sequence
  synthetic peptide
PEFSGQ

SEQ ID NO: 147 - protein, 6 aa, Artificial Sequence
  synthetic peptide
PEGYFQ

SEQ ID NO: 148 - protein, 6 aa, Artificial Sequence
  synthetic peptide
PESGFQ

SEQ ID NO: 149 - protein, 6 aa, Artificial Sequence
  synthetic peptide
PGYEFQ

SEQ ID NO: 150 - protein, 6 aa, Artificial Sequence
  synthetic peptide
PSGEFQ

SEQ ID NO: 151 - protein, 6 aa, Artificial Sequence
  synthetic peptide
PGEFYQ

SEQ ID NO: 152 - protein, 6 aa, Artificial Sequence
  synthetic peptide
PSEFGQ

SEQ ID NO: 153 - protein, 6 aa, Artificial Sequence
  synthetic peptide
PEKGYD

SEQ ID NO: 154 - protein, 6 aa, Artificial Sequence
  synthetic peptide
PEKSGD

SEQ ID NO: 155 - protein, 6 aa, Artificial Sequence
  synthetic peptide
PEGYKD

SEQ ID NO: 156 - protein, 6 aa, Artificial Sequence
  synthetic peptide
PESGKD

SEQ ID NO: 157 - protein, 6 aa, Artificial Sequence
  synthetic peptide
PGYEKD

SEQ ID NO: 158 - protein, 6 aa, Artificial Sequence
  synthetic peptide
PSGEKD

SEQ ID NO: 159 - protein, 6 aa, Artificial Sequence
  synthetic peptide
PGEKYD

SEQ ID NO: 160 - protein, 6 aa, Artificial Sequence
  synthetic peptide
PSEKGD

SEQ ID NO: 161 - protein, 6 aa, Artificial Sequence
  synthetic peptide
PQTGYE

SEQ ID NO: 162 - protein, 6 aa, Artificial Sequence
  synthetic peptide
PQTSGE

SEQ ID NO: 163 - protein, 6 aa, Artificial Sequence
  synthetic peptide
PQGYTE

SEQ ID NO: 164 - protein, 6 aa, Artificial Sequence
  synthetic peptide
PQSGTE

SEQ ID NO: 165 - protein, 6 aa, Artificial Sequence
  synthetic peptide
PGYQTE

SEQ ID NO: 166 - protein, 6 aa, Artificial Sequence
  synthetic peptide
PSGQTE

SEQ ID NO: 167 - protein, 6 aa, Artificial Sequence
  synthetic peptide
PGQTYE

SEQ ID NO: 168 - protein, 6 aa, Artificial Sequence
  synthetic peptide
PSQTGE

SEQ ID NO: 169 - protein, 6 aa, Artificial Sequence
  synthetic peptide
PNEGYF

SEQ ID NO: 170 - protein, 6 aa, Artificial Sequence
  synthetic peptide
PNESGF

SEQ ID NO: 171 - protein, 6 aa, Artificial Sequence
  synthetic peptide
PNGYEF

SEQ ID NO: 172 - protein, 6 aa, Artificial Sequence
  synthetic peptide
PNSGEF

SEQ ID NO: 173 - protein, 6 aa, Artificial Sequence
  synthetic peptide
PGYNEF

SEQ ID NO: 174 - protein, 6 aa, Artificial Sequence
  synthetic peptide
PSGNEF

SEQ ID NO: 175 - protein, 6 aa, Artificial Sequence
  synthetic peptide
PGNEYF

SEQ ID NO: 176 - protein, 6 aa, Artificial Sequence
  synthetic peptide
PSNEGF

SEQ ID NO: 177 - protein, 6 aa, Artificial Sequence
  synthetic peptide
PFEGYQ

SEQ ID NO: 178 - protein, 6 aa, Artificial Sequence
  synthetic peptide
PFESGQ

SEQ ID NO: 179 - protein, 6 aa, Artificial Sequence
  synthetic peptide
PFGYEQ

SEQ ID NO: 180 - protein, 6 aa, Artificial Sequence
  synthetic peptide
PFSGEQ

SEQ ID NO: 181 - protein, 6 aa, Artificial Sequence
  synthetic peptide
PGYFEQ

SEQ ID NO: 182 - protein, 6 aa, Artificial Sequence
  synthetic peptide
PSGFEQ

SEQ ID NO: 183 - protein, 6 aa, Artificial Sequence
  synthetic peptide
PGFEYQ

SEQ ID NO: 184 - protein, 6 aa, Artificial Sequence
  synthetic peptide
PSFEGQ

SEQ ID NO: 185 - protein, 6 aa, Artificial Sequence
  synthetic peptide
PFHGYL

SEQ ID NO: 186 - protein, 6 aa, Artificial Sequence
  synthetic peptide
PFHSGL

SEQ ID NO: 187 - protein, 6 aa, Artificial Sequence
  synthetic peptide
PFGYHL

SEQ ID NO: 188 - protein, 6 aa, Artificial Sequence
  synthetic peptide
PFSGHL

SEQ ID NO: 189 - protein, 6 aa, Artificial Sequence
  synthetic peptide
PGYFHL

SEQ ID NO: 190 - protein, 6 aa, Artificial Sequence
  synthetic peptide
PSGFHL

SEQ ID NO: 191 - protein, 6 aa, Artificial Sequence
  synthetic peptide
PGFHYL

SEQ ID NO: 192 - protein, 6 aa, Artificial Sequence
  synthetic peptide
PSFHGL

SEQ ID NO: 193 - protein, 6 aa, Artificial Sequence
  synthetic peptide
PHEGYK

SEQ ID NO: 194 - protein, 6 aa, Artificial Sequence
  synthetic peptide
PHESGK

SEQ ID NO: 195 - protein, 6 aa, Artificial Sequence
  synthetic peptide
PHGYEK

SEQ ID NO: 196 - protein, 6 aa, Artificial Sequence
  synthetic peptide
PHSGEK

SEQ ID NO: 197 - protein, 6 aa, Artificial Sequence
  synthetic peptide
PGYHEK

SEQ ID NO: 198 - protein, 6 aa, Artificial Sequence
  synthetic peptide
PSGHEK

SEQ ID NO: 199 - protein, 6 aa, Artificial Sequence
  synthetic peptide
PGHEYK

SEQ ID NO: 200 - protein, 6 aa, Artificial Sequence
  synthetic peptide
PSHEGK

SEQ ID NO: 201 - protein, 6 aa, Artificial Sequence
  synthetic peptide
PHTGYF

SEQ ID NO: 202 - protein, 6 aa, Artificial Sequence
  synthetic peptide
PHTSGF

SEQ ID NO: 203 - protein, 6 aa, Artificial Sequence
  synthetic peptide
PHGYTF

SEQ ID NO: 204 - protein, 6 aa, Artificial Sequence
  synthetic peptide
PHSGTF

SEQ ID NO: 205 - protein, 6 aa, Artificial Sequence
  synthetic peptide
PGYHTF

SEQ ID NO: 206 - protein, 6 aa, Artificial Sequence
  synthetic peptide
PSGHTF

SEQ ID NO: 207 - protein, 6 aa, Artificial Sequence
  synthetic peptide
PGHTYF

SEQ ID NO: 208 - protein, 6 aa, Artificial Sequence
  synthetic peptide
PSHTGF

SEQ ID NO: 209 - protein, 6 aa, Artificial Sequence
  synthetic peptide
PTLGYD

SEQ ID NO: 210 - protein, 6 aa, Artificial Sequence
  synthetic peptide
PTLSGD

SEQ ID NO: 211 - protein, 6 aa, Artificial Sequence
  synthetic peptide
PTGYLD

SEQ ID NO: 212 - protein, 6 aa, Artificial Sequence
  synthetic peptide
PTSGLD

SEQ ID NO: 213 - protein, 6 aa, Artificial Sequence
  synthetic peptide
PGYTLD

SEQ ID NO: 214 - protein, 6 aa, Artificial Sequence
  synthetic peptide
PSGTLD

SEQ ID NO: 215 - protein, 6 aa, Artificial Sequence
  synthetic peptide
PGTLYD

SEQ ID NO: 216 - protein, 6 aa, Artificial Sequence
  synthetic peptide
PSTLGD

SEQ ID NO: 217 - protein, 6 aa, Artificial Sequence
  synthetic peptide
PKHGYT

SEQ ID NO: 218 - protein, 6 aa, Artificial Sequence
  synthetic peptide
PKHSGT

SEQ ID NO: 219 - protein, 6 aa, Artificial Sequence
  synthetic peptide
PKGYHT

SEQ ID NO: 220 - protein, 6 aa, Artificial Sequence
  synthetic peptide
PKSGHT

SEQ ID NO: 221 - protein, 6 aa, Artificial Sequence
  synthetic peptide
PGYKHT

SEQ ID NO: 222 - protein, 6 aa, Artificial Sequence
  synthetic peptide
PSGKHT

SEQ ID NO: 223 - protein, 6 aa, Artificial Sequence
  synthetic peptide
PGKHYT

SEQ ID NO: 224 - protein, 6 aa, Artificial Sequence
  synthetic peptide
PSKHGT

SEQ ID NO: 225 - protein, 6 aa, Artificial Sequence
  synthetic peptide
PLDGYN

SEQ ID NO: 226 - protein, 6 aa, Artificial Sequence
  synthetic peptide
PLDSGN

SEQ ID NO: 227 - protein, 6 aa, Artificial Sequence
  synthetic peptide
PLGYDN

SEQ ID NO: 228 - protein, 6 aa, Artificial Sequence
  synthetic peptide
PLSGDN

SEQ ID NO: 229 - protein, 6 aa, Artificial Sequence
  synthetic peptide
PGYLDN

SEQ ID NO: 230 - protein, 6 aa, Artificial Sequence
  synthetic peptide
PSGLDN

SEQ ID NO: 231 - protein, 6 aa, Artificial Sequence
  synthetic peptide
PGLDYN

SEQ ID NO: 232 - protein, 6 aa, Artificial Sequence
  synthetic peptide
PSLDGN

SEQ ID NO: 233 - protein, 6 aa, Artificial Sequence
  synthetic peptide
QEPGYD

SEQ ID NO: 234 - protein, 6 aa, Artificial Sequence
  synthetic peptide
QEPSGD

SEQ ID NO: 235 - protein, 6 aa, Artificial Sequence
  synthetic peptide
QEGYPD

SEQ ID NO: 236 - protein, 6 aa, Artificial Sequence
  synthetic peptide
QESGPD

SEQ ID NO: 237 - protein, 6 aa, Artificial Sequence
  synthetic peptide
QGYEPD

SEQ ID NO: 238 - protein, 6 aa, Artificial Sequence
  synthetic peptide
QSGEPD

SEQ ID NO: 239 - protein, 6 aa, Artificial Sequence
  synthetic peptide
QGEPYD

SEQ ID NO: 240 - protein, 6 aa, Artificial Sequence
  synthetic peptide
QSEPGD

SEQ ID NO: 241 - protein, 6 aa, Artificial Sequence
  synthetic peptide
QETGYF

SEQ ID NO: 242 - protein, 6 aa, Artificial Sequence
  synthetic peptide
QETSGF

SEQ ID NO: 243 - protein, 6 aa, Artificial Sequence
  synthetic peptide
QEGYTF

SEQ ID NO: 244 - protein, 6 aa, Artificial Sequence
  synthetic peptide
QESGTF

SEQ ID NO: 245 - protein, 6 aa, Artificial Sequence
  synthetic peptide
QGYETF

SEQ ID NO: 246 - protein, 6 aa, Artificial Sequence
  synthetic peptide
QSGETF

SEQ ID NO: 247 - protein, 6 aa, Artificial Sequence
  synthetic peptide
QGETYF

SEQ ID NO: 248 - protein, 6 aa, Artificial Sequence
  synthetic peptide
QSETGF

SEQ ID NO: 249 - protein, 6 aa, Artificial Sequence
  synthetic peptide
QPEGYH

SEQ ID NO: 250 - protein, 6 aa, Artificial Sequence
  synthetic peptide
QPESGH

SEQ ID NO: 251 - protein, 6 aa, Artificial Sequence
  synthetic peptide
QPGYEH

SEQ ID NO: 252 - protein, 6 aa, Artificial Sequence
  synthetic peptide
QPSGEH

SEQ ID NO: 253 - protein, 6 aa, Artificial Sequence
  synthetic peptide
QGYPEH

SEQ ID NO: 254 - protein, 6 aa, Artificial Sequence
  synthetic peptide
QSGPEH

SEQ ID NO: 255 - protein, 6 aa, Artificial Sequence
  synthetic peptide
QGPEYH

SEQ ID NO: 256 - protein, 6 aa, Artificial Sequence
  synthetic peptide
QSPEGH

SEQ ID NO: 257 - protein, 6 aa, Artificial Sequence
  synthetic peptide
QNHGYE

SEQ ID NO: 258 - protein, 6 aa, Artificial Sequence
  synthetic peptide
QNHSGE

SEQ ID NO: 259 - protein, 6 aa, Artificial Sequence
  synthetic peptide
QNGYHE

SEQ ID NO: 260 - protein, 6 aa, Artificial Sequence
  synthetic peptide
QNSGHE

SEQ ID NO: 261 - protein, 6 aa, Artificial Sequence
  synthetic peptide
QGYNHE

SEQ ID NO: 262 - protein, 6 aa, Artificial Sequence
  synthetic peptide
QGYNHE

SEQ ID NO: 263 - protein, 6 aa, Artificial Sequence
  synthetic peptide
QGNHYE

SEQ ID NO: 264 - protein, 6 aa, Artificial Sequence
  synthetic peptide
QSNHGE

SEQ ID NO: 265 - protein, 6 aa, Artificial Sequence
  synthetic peptide
QFEGYK

SEQ ID NO: 266 - protein, 6 aa, Artificial Sequence
  synthetic peptide
QFESGK

SEQ ID NO: 267 - protein, 6 aa, Artificial Sequence
  synthetic peptide
QFGYEK

SEQ ID NO: 268 - protein, 6 aa, Artificial Sequence
  synthetic peptide
QFSGEK

SEQ ID NO: 269 - protein, 6 aa, Artificial Sequence
  synthetic peptide
QGYFEK

SEQ ID NO: 270 - protein, 6 aa, Artificial Sequence
  synthetic peptide
QSGFEK

SEQ ID NO: 271 - protein, 6 aa, Artificial Sequence
  synthetic peptide
QGFEYK

SEQ ID NO: 272 - protein, 6 aa, Artificial Sequence
  synthetic peptide
QSFEGK

SEQ ID NO: 273 - protein, 6 aa, Artificial Sequence
  synthetic peptide
QTFGYN

SEQ ID NO: 274 - protein, 6 aa, Artificial Sequence
  synthetic peptide
QTFSGN

SEQ ID NO: 275 - protein, 6 aa, Artificial Sequence
  synthetic peptide
QTGYFN

SEQ ID NO: 276 - protein, 6 aa, Artificial Sequence
  synthetic peptide
QTSGFN

SEQ ID NO: 277 - protein, 6 aa, Artificial Sequence
  synthetic peptide
QGYTFN

SEQ ID NO: 278 - protein, 6 aa, Artificial Sequence
  synthetic peptide
QSGTFN

SEQ ID NO: 279 - protein, 6 aa, Artificial Sequence
  synthetic peptide
QGTFYN

SEQ ID NO: 280 - protein, 6 aa, Artificial Sequence
  synthetic peptide
QSTFGN

SEQ ID NO: 281 - protein, 6 aa, Artificial Sequence
  synthetic peptide
QKEGYF

SEQ ID NO: 282 - protein, 6 aa, Artificial Sequence
  synthetic peptide
QKESGF

SEQ ID NO: 283 - protein, 6 aa, Artificial Sequence
  synthetic peptide
QKGYEF

SEQ ID NO: 284 - protein, 6 aa, Artificial Sequence
  synthetic peptide
QKSGEF

SEQ ID NO: 285 - protein, 6 aa, Artificial Sequence
  synthetic peptide
QGYKEF

SEQ ID NO: 286 - protein, 6 aa, Artificial Sequence
  synthetic peptide
QSGKEF

SEQ ID NO: 287 - protein, 6 aa, Artificial Sequence
  synthetic peptide
QGKEYF

SEQ ID NO: 288 - protein, 6 aa, Artificial Sequence
  synthetic peptide
QSKEGF

SEQ ID NO: 289 - protein, 6 aa, Artificial Sequence
  synthetic peptide
QLHGYT

SEQ ID NO: 290 - protein, 6 aa, Artificial Sequence
  synthetic peptide
QLHSGT

SEQ ID NO: 291 - protein, 6 aa, Artificial Sequence
  synthetic peptide
QLGYHT

SEQ ID NO: 292 - protein, 6 aa, Artificial Sequence
  synthetic peptide
QLSGHT

SEQ ID NO: 293 - protein, 6 aa, Artificial Sequence
  synthetic peptide
QGYLHT

SEQ ID NO: 294 - protein, 6 aa, Artificial Sequence
  synthetic peptide
QSGLHT

SEQ ID NO: 295 - protein, 6 aa, Artificial Sequence
  synthetic peptide
QGLHYT

SEQ ID NO: 296 - protein, 6 aa, Artificial Sequence
  synthetic peptide
QSLHGT

SEQ ID NO: 297 - protein, 6 aa, Artificial Sequence
  synthetic peptide
QLDGYE

SEQ ID NO: 298 - protein, 6 aa, Artificial Sequence
  synthetic peptide
QLDSGE

SEQ ID NO: 299 - protein, 6 aa, Artificial Sequence
  synthetic peptide
QLGYDE

SEQ ID NO: 300 - protein, 6 aa, Artificial Sequence
  synthetic peptide
QLSGDE

SEQ ID NO: 301 - protein, 6 aa, Artificial Sequence
  synthetic peptide
QGYLDE

SEQ ID NO: 302 - protein, 6 aa, Artificial Sequence
  synthetic peptide
QSGLDE

SEQ ID NO: 303 - protein, 6 aa, Artificial Sequence
  synthetic peptide
QGLDYE

SEQ ID NO: 304 - protein, 6 aa, Artificial Sequence
  synthetic peptide
QSLDGE

SEQ ID NO: 305 - protein, 6 aa, Artificial Sequence
  synthetic peptide
NEPGYL

SEQ ID NO: 306 - protein, 6 aa, Artificial Sequence
  synthetic peptide
NEPSGL

SEQ ID NO: 307 - protein, 6 aa, Artificial Sequence
  synthetic peptide
NEGYPL

SEQ ID NO: 308 - protein, 6 aa, Artificial Sequence
  synthetic peptide
NESGPL

SEQ ID NO: 309 - protein, 6 aa, Artificial Sequence
  synthetic peptide
NGYEPL

SEQ ID NO: 310 - protein, 6 aa, Artificial Sequence
  synthetic peptide
NSGEPL

SEQ ID NO: 311 - protein, 6 aa, Artificial Sequence
  synthetic peptide
NGEPYL

SEQ ID NO: 312 - protein, 6 aa, Artificial Sequence
  synthetic peptide
NSEPGL

SEQ ID NO: 313 - protein, 6 aa, Artificial Sequence
  synthetic peptide
NEFGYH

SEQ ID NO: 314 - protein, 6 aa, Artificial Sequence
  synthetic peptide
NEFSGH

SEQ ID NO: 315 - protein, 6 aa, Artificial Sequence
  synthetic peptide
NEGYFH

SEQ ID NO: 316 - protein, 6 aa, Artificial Sequence
  synthetic peptide
NESGFH

SEQ ID NO: 317 - protein, 6 aa, Artificial Sequence
  synthetic peptide
NGYEFH

SEQ ID NO: 318 - protein, 6 aa, Artificial Sequence
  synthetic peptide
NSGEFH

SEQ ID NO: 319 - protein, 6 aa, Artificial Sequence
  synthetic peptide
NGEFYH

SEQ ID NO: 320 - protein, 6 aa, Artificial Sequence
  synthetic peptide
NSEFGH

SEQ ID NO: 321 - protein, 6 aa, Artificial Sequence
  synthetic peptide
NPEGYF

SEQ ID NO: 322 - protein, 6 aa, Artificial Sequence
  synthetic peptide
NPESGF

SEQ ID NO: 323 - protein, 6 aa, Artificial Sequence
  synthetic peptide
NPGYEF

SEQ ID NO: 324 - protein, 6 aa, Artificial Sequence
  synthetic peptide
NPSGEF

SEQ ID NO: 325 - protein, 6 aa, Artificial Sequence
  synthetic peptide
NGYPEF

SEQ ID NO: 326 - protein, 6 aa, Artificial Sequence
  synthetic peptide
NSGPEF

SEQ ID NO: 327 - protein, 6 aa, Artificial Sequence
  synthetic peptide
NGPEYF

SEQ ID NO: 328 - protein, 6 aa, Artificial Sequence
  synthetic peptide
NSPEGF

SEQ ID NO: 329 - protein, 6 aa, Artificial Sequence
  synthetic peptide
NQHGYD

SEQ ID NO: 330 - protein, 6 aa, Artificial Sequence
  synthetic peptide
NQHSGD

SEQ ID NO: 331 - protein, 6 aa, Artificial Sequence
  synthetic peptide
NQGYHD

SEQ ID NO: 332 - protein, 6 aa, Artificial Sequence
  synthetic peptide
NQSGHD

SEQ ID NO: 333 - protein, 6 aa, Artificial Sequence
  synthetic peptide
NGYQHD

SEQ ID NO: 334 - protein, 6 aa, Artificial Sequence
  synthetic peptide
NSGQHD

SEQ ID NO: 335 - protein, 6 aa, Artificial Sequence
  synthetic peptide
NGQHYD

SEQ ID NO: 336 - protein, 6 aa, Artificial Sequence
  synthetic peptide
NSQHGD

SEQ ID NO: 337 - protein, 6 aa, Artificial Sequence
  synthetic peptide
NFEGYP

SEQ ID NO: 338 - protein, 6 aa, Artificial Sequence
  synthetic peptide
NFESGP

SEQ ID NO: 339 - protein, 6 aa, Artificial Sequence
  synthetic peptide
NFGYEP

SEQ ID NO: 340 - protein, 6 aa, Artificial Sequence
  synthetic peptide
NFSGEP

SEQ ID NO: 341 - protein, 6 aa, Artificial Sequence
  synthetic peptide
NGYFEP

SEQ ID NO: 342 - protein, 6 aa, Artificial Sequence
  synthetic peptide
NSGFEP

SEQ ID NO: 343 - protein, 6 aa, Artificial Sequence
  synthetic peptide
NGFEYP

SEQ ID NO: 344 - protein, 6 aa, Artificial Sequence
  synthetic peptide
NSFEGP

SEQ ID NO: 345 - protein, 6 aa, Artificial Sequence
  synthetic peptide
NFKGYH

SEQ ID NO: 346 - protein, 6 aa, Artificial Sequence
  synthetic peptide
NFKSGH

SEQ ID NO: 347 - protein, 6 aa, Artificial Sequence
  synthetic peptide
NFGYKH

SEQ ID NO: 348 - protein, 6 aa, Artificial Sequence
  synthetic peptide
NFSGKH

SEQ ID NO: 349 - protein, 6 aa, Artificial Sequence
  synthetic peptide
NGYFKH

SEQ ID NO: 350 - protein, 6 aa, Artificial Sequence
  synthetic peptide
NSGFKH

SEQ ID NO: 351 - protein, 6 aa, Artificial Sequence
  synthetic peptide
NGFKYH

SEQ ID NO: 352 - protein, 6 aa, Artificial Sequence
  synthetic peptide
NSFKGH

SEQ ID NO: 353 - protein, 6 aa, Artificial Sequence
  synthetic peptide
NHPGYT

SEQ ID NO: 354 - protein, 6 aa, Artificial Sequence
  synthetic peptide
NHPSGT

SEQ ID NO: 355 - protein, 6 aa, Artificial Sequence
  synthetic peptide
NHGYPT

SEQ ID NO: 356 - protein, 6 aa, Artificial Sequence
  synthetic peptide
NHSGPT

SEQ ID NO: 357 - protein, 6 aa, Artificial Sequence
  synthetic peptide
NGYHPT

SEQ ID NO: 358 - protein, 6 aa, Artificial Sequence
  synthetic peptide
NSGHPT

SEQ ID NO: 359 - protein, 6 aa, Artificial Sequence
  synthetic peptide
NGHPYT

SEQ ID NO: 360 - protein, 6 aa, Artificial Sequence
  synthetic peptide
NSHPGT

SEQ ID NO: 361 - protein, 6 aa, Artificial Sequence
  synthetic peptide
NHTGYD

SEQ ID NO: 362 - protein, 6 aa, Artificial Sequence
  synthetic peptide
NHTSGD

SEQ ID NO: 363 - protein, 6 aa, Artificial Sequence
  synthetic peptide
NHGYTD

SEQ ID NO: 364 - protein, 6 aa, Artificial Sequence
  synthetic peptide
NHSGTD

SEQ ID NO: 365 - protein, 6 aa, Artificial Sequence
  synthetic peptide
NGYHTD

SEQ ID NO: 366 - protein, 6 aa, Artificial Sequence
  synthetic peptide
NSGHTD

SEQ ID NO: 367 - protein, 6 aa, Artificial Sequence
  synthetic peptide
NGHTYD

SEQ ID NO: 368 - protein, 6 aa, Artificial Sequence
  synthetic peptide
NSHTGD

SEQ ID NO: 369 - protein, 6 aa, Artificial Sequence
  synthetic peptide
NTHGYK

SEQ ID NO: 370 - protein, 6 aa, Artificial Sequence
  synthetic peptide
NTHSGK

SEQ ID NO: 371 - protein, 6 aa, Artificial Sequence
  synthetic peptide
NTGYHK

SEQ ID NO: 372 - protein, 6 aa, Artificial Sequence
  synthetic peptide
NTSGHK

SEQ ID NO: 373 - protein, 6 aa, Artificial Sequence
  synthetic peptide
NGYTHK

SEQ ID NO: 374 - protein, 6 aa, Artificial Sequence
  synthetic peptide
NSGTHK

SEQ ID NO: 375 - protein, 6 aa, Artificial Sequence
  synthetic peptide
NGTHYK

SEQ ID NO: 376 - protein, 6 aa, Artificial Sequence
  synthetic peptide
NSTHGK

SEQ ID NO: 377 - protein, 6 aa, Artificial Sequence
  synthetic peptide
NKHGYL

SEQ ID NO: 378 - protein, 6 aa, Artificial Sequence
  synthetic peptide
NKHSGL

SEQ ID NO: 379 - protein, 6 aa, Artificial Sequence
  synthetic peptide
NKGYHL

SEQ ID NO: 380 - protein, 6 aa, Artificial Sequence
  synthetic peptide
NKSGHL

SEQ ID NO: 381 - protein, 6 aa, Artificial Sequence
  synthetic peptide
NGYKHL

SEQ ID NO: 382 - protein, 6 aa, Artificial Sequence
  synthetic peptide
NSGKHL

SEQ ID NO: 383 - protein, 6 aa, Artificial Sequence
  synthetic peptide
NGKHYL

SEQ ID NO: 384 - protein, 6 aa, Artificial Sequence
  synthetic peptide
NSKHGL

SEQ ID NO: 385 - protein, 6 aa, Artificial Sequence
  synthetic peptide
NLFGYD

SEQ ID NO: 386 - protein, 6 aa, Artificial Sequence
  synthetic peptide
NLFSGD

SEQ ID NO: 387 - protein, 6 aa, Artificial Sequence
  synthetic peptide
NLGYFD

SEQ ID NO: 388 - protein, 6 aa, Artificial Sequence
  synthetic peptide
NLSGFD

SEQ ID NO: 389 - protein, 6 aa, Artificial Sequence
  synthetic peptide
NGYLFD

SEQ ID NO: 390 - protein, 6 aa, Artificial Sequence
  synthetic peptide
NSGLFD

SEQ ID NO: 391 - protein, 6 aa, Artificial Sequence
  synthetic peptide
NGLFYD

SEQ ID NO: 392 - protein, 6 aa, Artificial Sequence
  synthetic peptide
NSLFGD

SEQ ID NO: 393 - protein, 6 aa, Artificial Sequence
  synthetic peptide
NDLGYF

SEQ ID NO: 394 - protein, 6 aa, Artificial Sequence
  synthetic peptide
NDLSGF

SEQ ID NO: 395 - protein, 6 aa, Artificial Sequence
  synthetic peptide
NDGYLF

SEQ ID NO: 396 - protein, 6 aa, Artificial Sequence
  synthetic peptide
NDSGLF

SEQ ID NO: 397 - protein, 6 aa, Artificial Sequence
  synthetic peptide
NGYDLF

SEQ ID NO: 398 - protein, 6 aa, Artificial Sequence
  synthetic peptide
NSGDLF

SEQ ID NO: 399 - protein, 6 aa, Artificial Sequence
  synthetic peptide
NGDLYF

SEQ ID NO: 400 - protein, 6 aa, Artificial Sequence
  synthetic peptide
NSDLGF

SEQ ID NO: 401 - protein, 6 aa, Artificial Sequence
  synthetic peptide
FEQGYP

SEQ ID NO: 402 - protein, 6 aa, Artificial Sequence
  synthetic peptide
FEQSGP

SEQ ID NO: 403 - protein, 6 aa, Artificial Sequence
  synthetic peptide
FEGYQP

SEQ ID NO: 404 - protein, 6 aa, Artificial Sequence
  synthetic peptide
FESGQP

SEQ ID NO: 405 - protein, 6 aa, Artificial Sequence
  synthetic peptide
FGYEQP

SEQ ID NO: 406 - protein, 6 aa, Artificial Sequence
  synthetic peptide
FSGEQP

SEQ ID NO: 407 - protein, 6 aa, Artificial Sequence
  synthetic peptide
FGEQYP

SEQ ID NO: 408 - protein, 6 aa, Artificial Sequence
  synthetic peptide
FSEQGP

SEQ ID NO: 409 - protein, 6 aa, Artificial Sequence
  synthetic peptide
FEKGYT

SEQ ID NO: 410 - protein, 6 aa, Artificial Sequence
  synthetic peptide
FEKSGT

SEQ ID NO: 411 - protein, 6 aa, Artificial Sequence
  synthetic peptide
FEGYKT

SEQ ID NO: 412 - protein, 6 aa, Artificial Sequence
  synthetic peptide
FESGKT

SEQ ID NO: 413 - protein, 6 aa, Artificial Sequence
  synthetic peptide
FGYEKT

SEQ ID NO: 414 - protein, 6 aa, Artificial Sequence
  synthetic peptide
FSGEKT

SEQ ID NO: 415 - protein, 6 aa, Artificial Sequence
  synthetic peptide
FGEKYT

SEQ ID NO: 416 - protein, 6 aa, Artificial Sequence
  synthetic peptide
FSEKGT

SEQ ID NO: 417 - protein, 6 aa, Artificial Sequence
  synthetic peptide
FEDGYH

SEQ ID NO: 418 - protein, 6 aa, Artificial Sequence
  synthetic peptide
FEDSGH

SEQ ID NO: 419 - protein, 6 aa, Artificial Sequence
  synthetic peptide
FEGYDH

SEQ ID NO: 420 - protein, 6 aa, Artificial Sequence
  synthetic peptide
FESGDH

SEQ ID NO: 421 - protein, 6 aa, Artificial Sequence
  synthetic peptide
FGYEDH

SEQ ID NO: 422 - protein, 6 aa, Artificial Sequence
  synthetic peptide
FSGEDH

SEQ ID NO: 423 - protein, 6 aa, Artificial Sequence
  synthetic peptide
FGEDYH

SEQ ID NO: 424 - protein, 6 aa, Artificial Sequence
  synthetic peptide
FSEDGH

SEQ ID NO: 425 - protein, 6 aa, Artificial Sequence
  synthetic peptide
FPNGYE

SEQ ID NO: 426 - protein, 6 aa, Artificial Sequence
  synthetic peptide
FPNSGE

SEQ ID NO: 427 - protein, 6 aa, Artificial Sequence
  synthetic peptide
FPGYNE

SEQ ID NO: 428 - protein, 6 aa, Artificial Sequence
  synthetic peptide
FPSGNE

SEQ ID NO: 429 - protein, 6 aa, Artificial Sequence
  synthetic peptide
FGYPNE

SEQ ID NO: 430 - protein, 6 aa, Artificial Sequence
  synthetic peptide
FSGPNE

SEQ ID NO: 431 - protein, 6 aa, Artificial Sequence
  synthetic peptide
FGPNYE

SEQ ID NO: 432 - protein, 6 aa, Artificial Sequence
  synthetic peptide
FSPNGE

SEQ ID NO: 433 - protein, 6 aa, Artificial Sequence
  synthetic peptide
FPKGYL

SEQ ID NO: 434 - protein, 6 aa, Artificial Sequence
  synthetic peptide
FPKSGL

SEQ ID NO: 435 - protein, 6 aa, Artificial Sequence
  synthetic peptide
FPGYKL

SEQ ID NO: 436 - protein, 6 aa, Artificial Sequence
  synthetic peptide
FPSGKL

SEQ ID NO: 437 - protein, 6 aa, Artificial Sequence
  synthetic peptide
FGYPKL

SEQ ID NO: 438 - protein, 6 aa, Artificial Sequence
  synthetic peptide
FSGPKL

SEQ ID NO: 439 - protein, 6 aa, Artificial Sequence
  synthetic peptide
FGPKYL

SEQ ID NO: 440 - protein, 6 aa, Artificial Sequence
  synthetic peptide
FSPKGL

SEQ ID NO: 441 - protein, 6 aa, Artificial Sequence
  synthetic peptide
FQNGYK

SEQ ID NO: 442 - protein, 6 aa, Artificial Sequence
  synthetic peptide
FQNSGK

SEQ ID NO: 443 - protein, 6 aa, Artificial Sequence
  synthetic peptide
FQGYNK

SEQ ID NO: 444 - protein, 6 aa, Artificial Sequence
  synthetic peptide
FQSGNK

SEQ ID NO: 445 - protein, 6 aa, Artificial Sequence
  synthetic peptide
FGYQNK

SEQ ID NO: 446 - protein, 6 aa, Artificial Sequence
  synthetic peptide
FSGQNK

SEQ ID NO: 447 - protein, 6 aa, Artificial Sequence
  synthetic peptide
FGQNYK

SEQ ID NO: 448 - protein, 6 aa, Artificial Sequence
  synthetic peptide
FSQNGK

SEQ ID NO: 449 - protein, 6 aa, Artificial Sequence
  synthetic peptide
FNPGYE

SEQ ID NO: 450 - protein, 6 aa, Artificial Sequence
  synthetic peptide
FNPSGE

SEQ ID NO: 451 - protein, 6 aa, Artificial Sequence
  synthetic peptide
FNGYPE

SEQ ID NO: 452 - protein, 6 aa, Artificial Sequence
  synthetic peptide
FNSGPE

SEQ ID NO: 453 - protein, 6 aa, Artificial Sequence
  synthetic peptide
FGYNPE

SEQ ID NO: 454 - protein, 6 aa, Artificial Sequence
  synthetic peptide
FSGNPE

SEQ ID NO: 455 - protein, 6 aa, Artificial Sequence
  synthetic peptide
FGNPYE

SEQ ID NO: 456 - protein, 6 aa, Artificial Sequence
  synthetic peptide
FSNPGE

SEQ ID NO: 457 - protein, 6 aa, Artificial Sequence
  synthetic peptide
FHEGYP

SEQ ID NO: 458 - protein, 6 aa, Artificial Sequence
  synthetic peptide
FHESGP

SEQ ID NO: 459 - protein, 6 aa, Artificial Sequence
  synthetic peptide
FHGYEP

SEQ ID NO: 460 - protein, 6 aa, Artificial Sequence
  synthetic peptide
FHSGEP

SEQ ID NO: 461 - protein, 6 aa, Artificial Sequence
  synthetic peptide
FGYHEP

SEQ ID NO: 462 - protein, 6 aa, Artificial Sequence
  synthetic peptide
FSGHEP

SEQ ID NO: 463 - protein, 6 aa, Artificial Sequence
  synthetic peptide
FGHEYP

SEQ ID NO: 464 - protein, 6 aa, Artificial Sequence
  synthetic peptide
FSHEGP

SEQ ID NO: 465 - protein, 6 aa, Artificial Sequence
  synthetic peptide
FHKGYE

SEQ ID NO: 466 - protein, 6 aa, Artificial Sequence
  synthetic peptide
FHKSGE

SEQ ID NO: 467 - protein, 6 aa, Artificial Sequence
  synthetic peptide
FHGYKE

SEQ ID NO: 468 - protein, 6 aa, Artificial Sequence
  synthetic peptide
FHSGKE

SEQ ID NO: 469 - protein, 6 aa, Artificial Sequence
  synthetic peptide
FGYHKE

SEQ ID NO: 470 - protein, 6 aa, Artificial Sequence
  synthetic peptide
FSGHKE

SEQ ID NO: 471 - protein, 6 aa, Artificial Sequence
  synthetic peptide
FGHKYE

SEQ ID NO: 472 - protein, 6 aa, Artificial Sequence
  synthetic peptide
FSHKGE

SEQ ID NO: 473 - protein, 6 aa, Artificial Sequence
  synthetic peptide
FTHGYN

SEQ ID NO: 474 - protein, 6 aa, Artificial Sequence
  synthetic peptide
FTHSGN

SEQ ID NO: 475 - protein, 6 aa, Artificial Sequence
  synthetic peptide
FTGYHN

SEQ ID NO: 476 - protein, 6 aa, Artificial Sequence
  synthetic peptide
FTSGHN

SEQ ID NO: 477 - protein, 6 aa, Artificial Sequence
  synthetic peptide
FGYTHN

SEQ ID NO: 478 - protein, 6 aa, Artificial Sequence
  synthetic peptide
FSGTHN

SEQ ID NO: 479 - protein, 6 aa, Artificial Sequence
  synthetic peptide
FGTHYN

SEQ ID NO: 480 - protein, 6 aa, Artificial Sequence
  synthetic peptide
FSTHGN

SEQ ID NO: 481 - protein, 6 aa, Artificial Sequence
  synthetic peptide
FTLGYQ

SEQ ID NO: 482 - protein, 6 aa, Artificial Sequence
  synthetic peptide
FTLSGQ

SEQ ID NO: 483 - protein, 6 aa, Artificial Sequence
  synthetic peptide
FTGYLQ

SEQ ID NO: 484 - protein, 6 aa, Artificial Sequence
  synthetic peptide
FTSGLQ

SEQ ID NO: 485 - protein, 6 aa, Artificial Sequence
  synthetic peptide
FGYTLQ

SEQ ID NO: 486 - protein, 6 aa, Artificial Sequence
  synthetic peptide
FSGTLQ

SEQ ID NO: 487 - protein, 6 aa, Artificial Sequence
  synthetic peptide
FGTLYQ

SEQ ID NO: 488 - protein, 6 aa, Artificial Sequence
  synthetic peptide
FSTLGQ

SEQ ID NO: 489 - protein, 6 aa, Artificial Sequence
  synthetic peptide
FKQGYH

SEQ ID NO: 490 - protein, 6 aa, Artificial Sequence
  synthetic peptide
FKQSGH

SEQ ID NO: 491 - protein, 6 aa, Artificial Sequence
  synthetic peptide
FKGYQH

SEQ ID NO: 492 - protein, 6 aa, Artificial Sequence
  synthetic peptide
FKSGQH

SEQ ID NO: 493 - protein, 6 aa, Artificial Sequence
  synthetic peptide
FGYKQH

SEQ ID NO: 494 - protein, 6 aa, Artificial Sequence
  synthetic peptide
FSGKQH

SEQ ID NO: 495 - protein, 6 aa, Artificial Sequence
  synthetic peptide
FGKQYH

SEQ ID NO: 496 - protein, 6 aa, Artificial Sequence
  synthetic peptide
FSKQGH

SEQ ID NO: 497 - protein, 6 aa, Artificial Sequence
  synthetic peptide
FKLGYP

SEQ ID NO: 498 - protein, 6 aa, Artificial Sequence
  synthetic peptide
FKLSGP

SEQ ID NO: 499 - protein, 6 aa, Artificial Sequence
  synthetic peptide
FKGYLP

SEQ ID NO: 500 - protein, 6 aa, Artificial Sequence
  synthetic peptide
FKSGLP

SEQ ID NO: 501 - protein, 6 aa, Artificial Sequence
  synthetic peptide
FGYKLP

SEQ ID NO: 502 - protein, 6 aa, Artificial Sequence
  synthetic peptide
FSGKLP

SEQ ID NO: 503 - protein, 6 aa, Artificial Sequence
  synthetic peptide
FGKLYP

SEQ ID NO: 504 - protein, 6 aa, Artificial Sequence
  synthetic peptide
FSKLGP

SEQ ID NO: 505 - protein, 6 aa, Artificial Sequence
  synthetic peptide
FLEGYD

SEQ ID NO: 506 - protein, 6 aa, Artificial Sequence
  synthetic peptide
FLESGD

SEQ ID NO: 507 - protein, 6 aa, Artificial Sequence
  synthetic peptide
FLGYED

SEQ ID NO: 508 - protein, 6 aa, Artificial Sequence
  synthetic peptide
FLSGED

SEQ ID NO: 509 - protein, 6 aa, Artificial Sequence
  synthetic peptide
FGYLED

SEQ ID NO: 510 - protein, 6 aa, Artificial Sequence
  synthetic peptide
FSGLED

SEQ ID NO: 511 - protein, 6 aa, Artificial Sequence
  synthetic peptide
FGLEYD

SEQ ID NO: 512 - protein, 6 aa, Artificial Sequence
  synthetic peptide
FSLEGD

SEQ ID NO: 513 - protein, 6 aa, Artificial Sequence
  synthetic peptide
FLHGYQ

SEQ ID NO: 514 - protein, 6 aa, Artificial Sequence
  synthetic peptide
FLHSGQ

SEQ ID NO: 515 - protein, 6 aa, Artificial Sequence
  synthetic peptide
FLGYHQ

SEQ ID NO: 516 - protein, 6 aa, Artificial Sequence
  synthetic peptide
FLSGHQ

SEQ ID NO: 517 - protein, 6 aa, Artificial Sequence
  synthetic peptide
FGYLHQ

SEQ ID NO: 518 - protein, 6 aa, Artificial Sequence
  synthetic peptide
FSGLHQ

SEQ ID NO: 519 - protein, 6 aa, Artificial Sequence
  synthetic peptide
FGLHYQ

SEQ ID NO: 520 - protein, 6 aa, Artificial Sequence
  synthetic peptide
FSLHGQ

SEQ ID NO: 521 - protein, 6 aa, Artificial Sequence
  synthetic peptide
FDTGYE

SEQ ID NO: 522 - protein, 6 aa, Artificial Sequence
  synthetic peptide
FDTSGE

SEQ ID NO: 523 - protein, 6 aa, Artificial Sequence
  synthetic peptide
FDGYTE

SEQ ID NO: 524 - protein, 6 aa, Artificial Sequence
  synthetic peptide
FDSGTE

SEQ ID NO: 525 - protein, 6 aa, Artificial Sequence
  synthetic peptide
FGYDTE

SEQ ID NO: 526 - protein, 6 aa, Artificial Sequence
  synthetic peptide
FSGDTE

SEQ ID NO: 527 - protein, 6 aa, Artificial Sequence
  synthetic peptide
FGDTYE

SEQ ID NO: 528 - protein, 6 aa, Artificial Sequence
  synthetic peptide
FSDTGE

SEQ ID NO: 529 - protein, 6 aa, Artificial Sequence
  synthetic peptide
HEQGYF

SEQ ID NO: 530 - protein, 6 aa, Artificial Sequence
  synthetic peptide
HEQSGF

SEQ ID NO: 531 - protein, 6 aa, Artificial Sequence
  synthetic peptide
HEGYQF

SEQ ID NO: 532 - protein, 6 aa, Artificial Sequence
  synthetic peptide
HESGQF

SEQ ID NO: 533 - protein, 6 aa, Artificial Sequence
  synthetic peptide
HGYEQF

SEQ ID NO: 534 - protein, 6 aa, Artificial Sequence
  synthetic peptide
HSGEQF

SEQ ID NO: 535 - protein, 6 aa, Artificial Sequence
  synthetic peptide
HGEQYF

SEQ ID NO: 536 - protein, 6 aa, Artificial Sequence
  synthetic peptide
HSEQGF

SEQ ID NO: 537 - protein, 6 aa, Artificial Sequence
  synthetic peptide
HEKGYP

SEQ ID NO: 538 - protein, 6 aa, Artificial Sequence
  synthetic peptide
HEKSGP

SEQ ID NO: 539 - protein, 6 aa, Artificial Sequence
  synthetic peptide
HEGYKP

SEQ ID NO: 540 - protein, 6 aa, Artificial Sequence
  synthetic peptide
HESGKP

SEQ ID NO: 541 - protein, 6 aa, Artificial Sequence
  synthetic peptide
HGYEKP

SEQ ID NO: 542 - protein, 6 aa, Artificial Sequence
  synthetic peptide
HSGEKP

SEQ ID NO: 543 - protein, 6 aa, Artificial Sequence
  synthetic peptide
HGEKYP

SEQ ID NO: 544 - protein, 6 aa, Artificial Sequence
  synthetic peptide
HSEKGP

SEQ ID NO: 545 - protein, 6 aa, Artificial Sequence
  synthetic peptide
HPEGYD

SEQ ID NO: 546 - protein, 6 aa, Artificial Sequence
  synthetic peptide
HPESGD

SEQ ID NO: 547 - protein, 6 aa, Artificial Sequence
  synthetic peptide
HPGYED

SEQ ID NO: 548 - protein, 6 aa, Artificial Sequence
  synthetic peptide
HPSGED

SEQ ID NO: 549 - protein, 6 aa, Artificial Sequence
  synthetic peptide
HGYPED

SEQ ID NO: 550 - protein, 6 aa, Artificial Sequence
  synthetic peptide
HSGPED

SEQ ID NO: 551 - protein, 6 aa, Artificial Sequence
  synthetic peptide
HGPEYD

SEQ ID NO: 552 - protein, 6 aa, Artificial Sequence
  synthetic peptide
HSPEGD

SEQ ID NO: 553 - protein, 6 aa, Artificial Sequence
  synthetic peptide
HPFGYL

SEQ ID NO: 554 - protein, 6 aa, Artificial Sequence
  synthetic peptide
HPFSGL

SEQ ID NO: 555 - protein, 6 aa, Artificial Sequence
  synthetic peptide
HPGYFL

SEQ ID NO: 556 - protein, 6 aa, Artificial Sequence
  synthetic peptide
HPSGFL

SEQ ID NO: 557 - protein, 6 aa, Artificial Sequence
  synthetic peptide
HGYPFL

SEQ ID NO: 558 - protein, 6 aa, Artificial Sequence
  synthetic peptide
HSGPFL

SEQ ID NO: 559 - protein, 6 aa, Artificial Sequence
  synthetic peptide
HGPFYL

SEQ ID NO: 560 - protein, 6 aa, Artificial Sequence
  synthetic peptide
HSPFGL

SEQ ID NO: 561 - protein, 6 aa, Artificial Sequence
  synthetic peptide
HQEGYL

SEQ ID NO: 562 - protein, 6 aa, Artificial Sequence
  synthetic peptide
HQESGL

SEQ ID NO: 563 - protein, 6 aa, Artificial Sequence
  synthetic peptide
HQGYEL

SEQ ID NO: 564 - protein, 6 aa, Artificial Sequence
  synthetic peptide
HQSGEL

SEQ ID NO: 565 - protein, 6 aa, Artificial Sequence
  synthetic peptide
HGYQEL

SEQ ID NO: 566 - protein, 6 aa, Artificial Sequence
  synthetic peptide
HSGQEL

SEQ ID NO: 567 - protein, 6 aa, Artificial Sequence
  synthetic peptide
HGQEYL

SEQ ID NO: 568 - protein, 6 aa, Artificial Sequence
  synthetic peptide
HSQEGL

SEQ ID NO: 569 - protein, 6 aa, Artificial Sequence
  synthetic peptide
HQTGYN

SEQ ID NO: 570 - protein, 6 aa, Artificial Sequence
  synthetic peptide
HQTSGN

SEQ ID NO: 571 - protein, 6 aa, Artificial Sequence
  synthetic peptide
HQGYTN

SEQ ID NO: 572 - protein, 6 aa, Artificial Sequence
  synthetic peptide
HQSGTN

SEQ ID NO: 573 - protein, 6 aa, Artificial Sequence
  synthetic peptide
HGYQTN

SEQ ID NO: 574 - protein, 6 aa, Artificial Sequence
  synthetic peptide
HSGQTN

SEQ ID NO: 575 - protein, 6 aa, Artificial Sequence
  synthetic peptide
HGQTYN

SEQ ID NO: 576 - protein, 6 aa, Artificial Sequence
  synthetic peptide
HSQTGN

SEQ ID NO: 577 - protein, 6 aa, Artificial Sequence
  synthetic peptide
HNKGYD

SEQ ID NO: 578 - protein, 6 aa, Artificial Sequence
  synthetic peptide
HNKSGD

SEQ ID NO: 579 - protein, 6 aa, Artificial Sequence
  synthetic peptide
HNGYKD

SEQ ID NO: 580 - protein, 6 aa, Artificial Sequence
  synthetic peptide
HNSGKD

SEQ ID NO: 581 - protein, 6 aa, Artificial Sequence
  synthetic peptide
HGYNKD

SEQ ID NO: 582 - protein, 6 aa, Artificial Sequence
  synthetic peptide
HSGNKD

SEQ ID NO: 583 - protein, 6 aa, Artificial Sequence
  synthetic peptide
HGNKYD

SEQ ID NO: 584 - protein, 6 aa, Artificial Sequence
  synthetic peptide
HSNKGD

SEQ ID NO: 585 - protein, 6 aa, Artificial Sequence
  synthetic peptide
HNDGYT

SEQ ID NO: 586 - protein, 6 aa, Artificial Sequence
  synthetic peptide
HNDSGT

SEQ ID NO: 587 - protein, 6 aa, Artificial Sequence
  synthetic peptide
HNGYDT

SEQ ID NO: 588 - protein, 6 aa, Artificial Sequence
  synthetic peptide
HNSGDT

SEQ ID NO: 589 - protein, 6 aa, Artificial Sequence
  synthetic peptide
HGYNDT

SEQ ID NO: 590 - protein, 6 aa, Artificial Sequence
  synthetic peptide
HSGNDT

SEQ ID NO: 591 - protein, 6 aa, Artificial Sequence
  synthetic peptide
HGNDYT

SEQ ID NO: 592 - protein, 6 aa, Artificial Sequence
  synthetic peptide
HSNDGT

SEQ ID NO: 593 - protein, 6 aa, Artificial Sequence
  synthetic peptide
HFTGYK

SEQ ID NO: 594 - protein, 6 aa, Artificial Sequence
  synthetic peptide
HFTSGK

SEQ ID NO: 595 - protein, 6 aa, Artificial Sequence
  synthetic peptide
HFGYTK

SEQ ID NO: 596 - protein, 6 aa, Artificial Sequence
  synthetic peptide
HFSGTK

SEQ ID NO: 597 - protein, 6 aa, Artificial Sequence
  synthetic peptide
HGYFTK

SEQ ID NO: 598 - protein, 6 aa, Artificial Sequence
  synthetic peptide
HSGFTK

SEQ ID NO: 599 - protein, 6 aa, Artificial Sequence
  synthetic peptide
HGFTYK

SEQ ID NO: 600 - protein, 6 aa, Artificial Sequence
  synthetic peptide
HSFTGK

SEQ ID NO: 601 - protein, 6 aa, Artificial Sequence
  synthetic peptide
HTPGYN

SEQ ID NO: 602 - protein, 6 aa, Artificial Sequence
  synthetic peptide
HTPSGN

SEQ ID NO: 603 - protein, 6 aa, Artificial Sequence
  synthetic peptide
HTGYPN

SEQ ID NO: 604 - protein, 6 aa, Artificial Sequence
  synthetic peptide
HTSGPN

SEQ ID NO: 605 - protein, 6 aa, Artificial Sequence
  synthetic peptide
HGYTPN

SEQ ID NO: 606 - protein, 6 aa, Artificial Sequence
  synthetic peptide
HSGTPN

SEQ ID NO: 607 - protein, 6 aa, Artificial Sequence
  synthetic peptide
HGTPYN

SEQ ID NO: 608 - protein, 6 aa, Artificial Sequence
  synthetic peptide
HSTPGN

SEQ ID NO: 609 - protein, 6 aa, Artificial Sequence
  synthetic peptide
HTFGYQ

SEQ ID NO: 610 - protein, 6 aa, Artificial Sequence
  synthetic peptide
HTFSGQ

SEQ ID NO: 611 - protein, 6 aa, Artificial Sequence
  synthetic peptide
HTGYFQ

SEQ ID NO: 612 - protein, 6 aa, Artificial Sequence
  synthetic peptide
HTSGFQ

SEQ ID NO: 613 - protein, 6 aa, Artificial Sequence
  synthetic peptide
HGYTFQ

SEQ ID NO: 614 - protein, 6 aa, Artificial Sequence
  synthetic peptide
HSGTFQ

SEQ ID NO: 615 - protein, 6 aa, Artificial Sequence
  synthetic peptide
HGTFYQ

SEQ ID NO: 616 - protein, 6 aa, Artificial Sequence
  synthetic peptide
HSTFGQ

SEQ ID NO: 617 - protein, 6 aa, Artificial Sequence
  synthetic peptide
HKPGYE

SEQ ID NO: 618 - protein, 6 aa, Artificial Sequence
  synthetic peptide
HKPSGE

SEQ ID NO: 619 - protein, 6 aa, Artificial Sequence
  synthetic peptide
HKGYPE

SEQ ID NO: 620 - protein, 6 aa, Artificial Sequence
  synthetic peptide
HKSGPE

SEQ ID NO: 621 - protein, 6 aa, Artificial Sequence
  synthetic peptide
HGYKPE

SEQ ID NO: 622 - protein, 6 aa, Artificial Sequence
  synthetic peptide
HSGKPE

SEQ ID NO: 623 - protein, 6 aa, Artificial Sequence
  synthetic peptide
HGKPYE

SEQ ID NO: 624 - protein, 6 aa, Artificial Sequence
  synthetic peptide
HSKPGE

SEQ ID NO: 625 - protein, 6 aa, Artificial Sequence
  synthetic peptide
HLEGYF

SEQ ID NO: 626 - protein, 6 aa, Artificial Sequence
  synthetic peptide
HLESGF

SEQ ID NO: 627 - protein, 6 aa, Artificial Sequence
  synthetic peptide
HLGYEF

SEQ ID NO: 628 - protein, 6 aa, Artificial Sequence
  synthetic peptide
HLSGEF

SEQ ID NO: 629 - protein, 6 aa, Artificial Sequence
  synthetic peptide
HGYLEF

SEQ ID NO: 630 - protein, 6 aa, Artificial Sequence
  synthetic peptide
HSGLEF

SEQ ID NO: 631 - protein, 6 aa, Artificial Sequence
  synthetic peptide
HGLEYF

SEQ ID NO: 632 - protein, 6 aa, Artificial Sequence
  synthetic peptide
HSLEGF

SEQ ID NO: 633 - protein, 6 aa, Artificial Sequence
  synthetic peptide
HDTGYL

SEQ ID NO: 634 - protein, 6 aa, Artificial Sequence
  synthetic peptide
HDTSGL

SEQ ID NO: 635 - protein, 6 aa, Artificial Sequence
  synthetic peptide
HDGYTL

SEQ ID NO: 636 - protein, 6 aa, Artificial Sequence
  synthetic peptide
HDSGTL

SEQ ID NO: 637 - protein, 6 aa, Artificial Sequence
  synthetic peptide
HGYDTL

SEQ ID NO: 638 - protein, 6 aa, Artificial Sequence
  synthetic peptide
HSGDTL

SEQ ID NO: 639 - protein, 6 aa, Artificial Sequence
  synthetic peptide
HGDTYL

SEQ ID NO: 640 - protein, 6 aa, Artificial Sequence
  synthetic peptide
HSDTGL

SEQ ID NO: 641 - protein, 6 aa, Artificial Sequence
  synthetic peptide
TEFGYL

SEQ ID NO: 642 - protein, 6 aa, Artificial Sequence
  synthetic peptide
TEFSGL

SEQ ID NO: 643 - protein, 6 aa, Artificial Sequence
  synthetic peptide
TEGYFL

SEQ ID NO: 644 - protein, 6 aa, Artificial Sequence
  synthetic peptide
TESGFL

SEQ ID NO: 645 - protein, 6 aa, Artificial Sequence
  synthetic peptide
TGYEFL

SEQ ID NO: 646 - protein, 6 aa, Artificial Sequence
  synthetic peptide
TSGEFL

SEQ ID NO: 647 - protein, 6 aa, Artificial Sequence
  synthetic peptide
TGEFYL

SEQ ID NO: 648 - protein, 6 aa, Artificial Sequence
  synthetic peptide
TSEFGL

SEQ ID NO: 649 - protein, 6 aa, Artificial Sequence
  synthetic peptide
TPDGYK

SEQ ID NO: 650 - protein, 6 aa, Artificial Sequence
  synthetic peptide
TPDSGK

SEQ ID NO: 651 - protein, 6 aa, Artificial Sequence
  synthetic peptide
TPGYDK

SEQ ID NO: 652 - protein, 6 aa, Artificial Sequence
  synthetic peptide
TPSGDK

SEQ ID NO: 653 - protein, 6 aa, Artificial Sequence
  synthetic peptide
TGYPDK

SEQ ID NO: 654 - protein, 6 aa, Artificial Sequence
  synthetic peptide
TSGPDK

SEQ ID NO: 655 - protein, 6 aa, Artificial Sequence
  synthetic peptide
TGPDYK

SEQ ID NO: 656 - protein, 6 aa, Artificial Sequence
  synthetic peptide
TSPDGK

SEQ ID NO: 657 - protein, 6 aa, Artificial Sequence
  synthetic peptide
TQLGYE

SEQ ID NO: 658 - protein, 6 aa, Artificial Sequence
  synthetic peptide
TQLSGE

SEQ ID NO: 659 - protein, 6 aa, Artificial Sequence
  synthetic peptide
TQGYLE

SEQ ID NO: 660 - protein, 6 aa, Artificial Sequence
  synthetic peptide
TQSGLE

SEQ ID NO: 661 - protein, 6 aa, Artificial Sequence
  synthetic peptide
TGYQLE

SEQ ID NO: 662 - protein, 6 aa, Artificial Sequence
  synthetic peptide
TSGQLE

SEQ ID NO: 663 - protein, 6 aa, Artificial Sequence
  synthetic peptide
TGQLYE

SEQ ID NO: 664 - protein, 6 aa, Artificial Sequence
  synthetic peptide
TSQLGE

SEQ ID NO: 665 - protein, 6 aa, Artificial Sequence
  synthetic peptide
TNDGYL

SEQ ID NO: 666 - protein, 6 aa, Artificial Sequence
  synthetic peptide
TNDSGL

SEQ ID NO: 667 - protein, 5 aa, Artificial Sequence
  synthetic peptide
TNGYD

SEQ ID NO: 668 - protein, 6 aa, Artificial Sequence
  synthetic peptide
TNSGDL

SEQ ID NO: 669 - protein, 6 aa, Artificial Sequence
  synthetic peptide
TGYNDL

SEQ ID NO: 670 - protein, 6 aa, Artificial Sequence
  synthetic peptide
TSGNDL

SEQ ID NO: 671 - protein, 6 aa, Artificial Sequence
  synthetic peptide
TGNDYL

SEQ ID NO: 672 - protein, 6 aa, Artificial Sequence
  synthetic peptide
TSNDGL

SEQ ID NO: 673 - protein, 6 aa, Artificial Sequence
  synthetic peptide
TFHGYE

SEQ ID NO: 674 - protein, 6 aa, Artificial Sequence
  synthetic peptide
TFHSGE

SEQ ID NO: 675 - protein, 6 aa, Artificial Sequence
  synthetic peptide
TFGYHE

SEQ ID NO: 676 - protein, 6 aa, Artificial Sequence
  synthetic peptide
TFSGHE

SEQ ID NO: 677 - protein, 6 aa, Artificial Sequence
  synthetic peptide
TGYFHE

SEQ ID NO: 678 - protein, 6 aa, Artificial Sequence
  synthetic peptide
TSGFHE

SEQ ID NO: 679 - protein, 6 aa, Artificial Sequence
  synthetic peptide
TGFHYE

SEQ ID NO: 680 - protein, 6 aa, Artificial Sequence
  synthetic peptide
TSFHGE

SEQ ID NO: 681 - protein, 6 aa, Artificial Sequence
  synthetic peptide
THLGYK

SEQ ID NO: 682 - protein, 6 aa, Artificial Sequence
  synthetic peptide
THLSGK

SEQ ID NO: 683 - protein, 6 aa, Artificial Sequence
  synthetic peptide
THGYLK

SEQ ID NO: 684 - protein, 6 aa, Artificial Sequence
  synthetic peptide
THSGLK

SEQ ID NO: 685 - protein, 6 aa, Artificial Sequence
  synthetic peptide
TGYHLK

SEQ ID NO: 686 - protein, 6 aa, Artificial Sequence
  synthetic peptide
TSGHLK

SEQ ID NO: 687 - protein, 6 aa, Artificial Sequence
  synthetic peptide
TGHLYK

SEQ ID NO: 688 - protein, 6 aa, Artificial Sequence
  synthetic peptide
TSHLGK

SEQ ID NO: 689 - protein, 6 aa, Artificial Sequence
  synthetic peptide
TLNGYF

SEQ ID NO: 690 - protein, 6 aa, Artificial Sequence
  synthetic peptide
TLNSGF

SEQ ID NO: 691 - protein, 6 aa, Artificial Sequence
  synthetic peptide
TLGYNF

SEQ ID NO: 692 - protein, 6 aa, Artificial Sequence
  synthetic peptide
TLSGNF

SEQ ID NO: 693 - protein, 6 aa, Artificial Sequence
  synthetic peptide
TGYLNF

SEQ ID NO: 694 - protein, 6 aa, Artificial Sequence
  synthetic peptide
TSGLNF

SEQ ID NO: 695 - protein, 6 aa, Artificial Sequence
  synthetic peptide
TGLNYF

SEQ ID NO: 696 - protein, 6 aa, Artificial Sequence
  synthetic peptide
TSLNGF

SEQ ID NO: 697 - protein, 6 aa, Artificial Sequence
  synthetic peptide
TDEGYQ

SEQ ID NO: 698 - protein, 6 aa, Artificial Sequence
  synthetic peptide
TDESGQ

SEQ ID NO: 699 - protein, 6 aa, Artificial Sequence
  synthetic peptide
TDGYEQ

SEQ ID NO: 700 - protein, 6 aa, Artificial Sequence
  synthetic peptide
TDSGEQ

SEQ ID NO: 701 - protein, 6 aa, Artificial Sequence
  synthetic peptide
TGYDEQ

SEQ ID NO: 702 - protein, 6 aa, Artificial Sequence
  synthetic peptide
TSGDEQ

SEQ ID NO: 703 - protein, 6 aa, Artificial Sequence
  synthetic peptide
TGDEYQ

SEQ ID NO: 704 - protein, 6 aa, Artificial Sequence
  synthetic peptide
TSDEGQ

SEQ ID NO: 705 - protein, 6 aa, Artificial Sequence
  synthetic peptide
KEPGYH

SEQ ID NO: 706 - protein, 6 aa, Artificial Sequence
  synthetic peptide
KEPSGH

SEQ ID NO: 707 - protein, 6 aa, Artificial Sequence
  synthetic peptide
KEGYPH

SEQ ID NO: 708 - protein, 6 aa, Artificial Sequence
  synthetic peptide
KESGPH

SEQ ID NO: 709 - protein, 6 aa, Artificial Sequence
  synthetic peptide
KGYEPH

SEQ ID NO: 710 - protein, 6 aa, Artificial Sequence
  synthetic peptide
KSGEPH

SEQ ID NO: 711 - protein, 6 aa, Artificial Sequence
  synthetic peptide
KGEPYH

SEQ ID NO: 712 - protein, 6 aa, Artificial Sequence
  synthetic peptide
KSEPGH

SEQ ID NO: 713 - protein, 6 aa, Artificial Sequence
  synthetic peptide
KEDGYF

SEQ ID NO: 714 - protein, 6 aa, Artificial Sequence
  synthetic peptide
KEDSGF

SEQ ID NO: 715 - protein, 6 aa, Artificial Sequence
  synthetic peptide
KEGYDF

SEQ ID NO: 716 - protein, 6 aa, Artificial Sequence
  synthetic peptide
KESGDF

SEQ ID NO: 717 - protein, 6 aa, Artificial Sequence
  synthetic peptide
KGYEDF

SEQ ID NO: 718 - protein, 6 aa, Artificial Sequence
  synthetic peptide
KSGEDF

SEQ ID NO: 719 - protein, 6 aa, Artificial Sequence
  synthetic peptide
KGEDYF

SEQ ID NO: 720 - protein, 6 aa, Artificial Sequence
  synthetic peptide
KSEDGF

SEQ ID NO: 721 - protein, 6 aa, Artificial Sequence
  synthetic peptide
KPHGYN

SEQ ID NO: 722 - protein, 6 aa, Artificial Sequence
  synthetic peptide
KPHSGN

SEQ ID NO: 723 - protein, 6 aa, Artificial Sequence
  synthetic peptide
KPGYHN

SEQ ID NO: 724 - protein, 6 aa, Artificial Sequence
  synthetic peptide
KPSGHN

SEQ ID NO: 725 - protein, 6 aa, Artificial Sequence
  synthetic peptide
KGYPHN

SEQ ID NO: 726 - protein, 6 aa, Artificial Sequence
  synthetic peptide
KSGPHN

SEQ ID NO: 727 - protein, 6 aa, Artificial Sequence
  synthetic peptide
KGPHYN

SEQ ID NO: 728 - protein, 6 aa, Artificial Sequence
  synthetic peptide
KSPHGN

SEQ ID NO: 729 - protein, 6 aa, Artificial Sequence
  synthetic peptide
KQNGYT

SEQ ID NO: 730 - protein, 6 aa, Artificial Sequence
  synthetic peptide
KQNSGT

SEQ ID NO: 731 - protein, 6 aa, Artificial Sequence
  synthetic peptide
KQGYNT

SEQ ID NO: 732 - protein, 6 aa, Artificial Sequence
  synthetic peptide
KQSGNT

SEQ ID NO: 733 - protein, 6 aa, Artificial Sequence
  synthetic peptide
KGYQNT

SEQ ID NO: 734 - protein, 6 aa, Artificial Sequence
  synthetic peptide
KSGQNT

SEQ ID NO: 735 - protein, 6 aa, Artificial Sequence
  synthetic peptide
KGQNYT

SEQ ID NO: 736 - protein, 6 aa, Artificial Sequence
  synthetic peptide
KSQNGT

SEQ ID NO: 737 - protein, 6 aa, Artificial Sequence
  synthetic peptide
KNPGYL

SEQ ID NO: 738 - protein, 6 aa, Artificial Sequence
  synthetic peptide
KNPSGL

SEQ ID NO: 739 - protein, 6 aa, Artificial Sequence
  synthetic peptide
KNGYPL

SEQ ID NO: 740 - protein, 6 aa, Artificial Sequence
  synthetic peptide
KNSGPL

SEQ ID NO: 741 - protein, 6 aa, Artificial Sequence
  synthetic peptide
KGYNPL

SEQ ID NO: 742 - protein, 6 aa, Artificial Sequence
  synthetic peptide
KSGNPL

SEQ ID NO: 743 - protein, 6 aa, Artificial Sequence
  synthetic peptide
KGNPYL

SEQ ID NO: 744 - protein, 6 aa, Artificial Sequence
  synthetic peptide
KSNPGL

SEQ ID NO: 745 - protein, 6 aa, Artificial Sequence
  synthetic peptide
KNDGYQ

SEQ ID NO: 746 - protein, 6 aa, Artificial Sequence
  synthetic peptide
KNDSGQ

SEQ ID NO: 747 - protein, 6 aa, Artificial Sequence
  synthetic peptide
KNGYDQ

SEQ ID NO: 748 - protein, 6 aa, Artificial Sequence
  synthetic peptide
KNSGDQ

SEQ ID NO: 749 - protein, 6 aa, Artificial Sequence
  synthetic peptide
KGYNDQ

SEQ ID NO: 750 - protein, 6 aa, Artificial Sequence
  synthetic peptide
KSGNDQ

SEQ ID NO: 751 - protein, 6 aa, Artificial Sequence
  synthetic peptide
KGNDYQ

SEQ ID NO: 752 - protein, 6 aa, Artificial Sequence
  synthetic peptide
KSNDGQ

SEQ ID NO: 753 - protein, 6 aa, Artificial Sequence
  synthetic peptide
KFHGYP

SEQ ID NO: 754 - protein, 6 aa, Artificial Sequence
  synthetic peptide
KFHSGP

SEQ ID NO: 755 - protein, 6 aa, Artificial Sequence
  synthetic peptide
KFGYHP

SEQ ID NO: 756 - protein, 6 aa, Artificial Sequence
  synthetic peptide
KFSGHP

SEQ ID NO: 757 - protein, 6 aa, Artificial Sequence
  synthetic peptide
KGYFHP

SEQ ID NO: 758 - protein, 6 aa, Artificial Sequence
  synthetic peptide
KSGFHP

SEQ ID NO: 759 - protein, 6 aa, Artificial Sequence
  synthetic peptide
KGFHYP

SEQ ID NO: 760 - protein, 6 aa, Artificial Sequence
  synthetic peptide
KSFHGP

SEQ ID NO: 761 - protein, 6 aa, Artificial Sequence
  synthetic peptide
KFLGYH

SEQ ID NO: 762 - protein, 6 aa, Artificial Sequence
  synthetic peptide
KFLSGH

SEQ ID NO: 763 - protein, 6 aa, Artificial Sequence
  synthetic peptide
KFGYLH

SEQ ID NO: 764 - protein, 6 aa, Artificial Sequence
  synthetic peptide
KFSGLH

SEQ ID NO: 765 - protein, 6 aa, Artificial Sequence
  synthetic peptide
KGYFLH

SEQ ID NO: 766 - protein, 6 aa, Artificial Sequence
  synthetic peptide
KSGFLH

SEQ ID NO: 767 - protein, 6 aa, Artificial Sequence
  synthetic peptide
KGFLYH

SEQ ID NO: 768 - protein, 6 aa, Artificial Sequence
  synthetic peptide
KSFLGH

SEQ ID NO: 769 - protein, 6 aa, Artificial Sequence
  synthetic peptide
KHPGYD

SEQ ID NO: 770 - protein, 6 aa, Artificial Sequence
  synthetic peptide
KHPSGD

SEQ ID NO: 771 - protein, 6 aa, Artificial Sequence
  synthetic peptide
KHGYPD

SEQ ID NO: 772 - protein, 6 aa, Artificial Sequence
  synthetic peptide
KHSGPD

SEQ ID NO: 773 - protein, 6 aa, Artificial Sequence
  synthetic peptide
KGYHPD

SEQ ID NO: 774 - protein, 6 aa, Artificial Sequence
  synthetic peptide
KSGHPD

SEQ ID NO: 775 - protein, 6 aa, Artificial Sequence
  synthetic peptide
KGHPYD

SEQ ID NO: 776 - protein, 6 aa, Artificial Sequence
  synthetic peptide
KSHPGD

SEQ ID NO: 777 - protein, 6 aa, Artificial Sequence
  synthetic peptide
KTNGYD

SEQ ID NO: 778 - protein, 6 aa, Artificial Sequence
  synthetic peptide
KTNSGD

SEQ ID NO: 779 - protein, 6 aa, Artificial Sequence
  synthetic peptide
KTGYND

SEQ ID NO: 780 - protein, 6 aa, Artificial Sequence
  synthetic peptide
KTSGND

SEQ ID NO: 781 - protein, 6 aa, Artificial Sequence
  synthetic peptide
KGYTND

SEQ ID NO: 782 - protein, 6 aa, Artificial Sequence
  synthetic peptide
KSGTND

SEQ ID NO: 783 - protein, 6 aa, Artificial Sequence
  synthetic peptide
KGTNYD

SEQ ID NO: 784 - protein, 6 aa, Artificial Sequence
  synthetic peptide
KSTNGD

SEQ ID NO: 785 - protein, 6 aa, Artificial Sequence
  synthetic peptide
KDNGYL

SEQ ID NO: 786 - protein, 6 aa, Artificial Sequence
  synthetic peptide
KDNSGL

SEQ ID NO: 787 - protein, 6 aa, Artificial Sequence
  synthetic peptide
KDGYNL

SEQ ID NO: 788 - protein, 6 aa, Artificial Sequence
  synthetic peptide
KDSGNL

SEQ ID NO: 789 - protein, 6 aa, Artificial Sequence
  synthetic peptide
KGYDNL

SEQ ID NO: 790 - protein, 6 aa, Artificial Sequence
  synthetic peptide
KSGDNL

SEQ ID NO: 791 - protein, 6 aa, Artificial Sequence
  synthetic peptide
KGDNYL

SEQ ID NO: 792 - protein, 6 aa, Artificial Sequence
  synthetic peptide
KSDNGL

SEQ ID NO: 793 - protein, 6 aa, Artificial Sequence
  synthetic peptide
KDHGYE

SEQ ID NO: 794 - protein, 6 aa, Artificial Sequence
  synthetic peptide
KDHSGE

SEQ ID NO: 795 - protein, 6 aa, Artificial Sequence
  synthetic peptide
KDGYHE

SEQ ID NO: 796 - protein, 6 aa, Artificial Sequence
  synthetic peptide
KDSGHE

SEQ ID NO: 797 - protein, 6 aa, Artificial Sequence
  synthetic peptide
KGYDHE

SEQ ID NO: 798 - protein, 6 aa, Artificial Sequence
  synthetic peptide
KSGDHE

SEQ ID NO: 799 - protein, 6 aa, Artificial Sequence
  synthetic peptide
KGDHYE

SEQ ID NO: 800 - protein, 6 aa, Artificial Sequence
  synthetic peptide
KSDHGE

SEQ ID NO: 801 - protein, 6 aa, Artificial Sequence
  synthetic peptide
LEFGYK

SEQ ID NO: 802 - protein, 6 aa, Artificial Sequence
  synthetic peptide
LEFSGK

SEQ ID NO: 803 - protein, 6 aa, Artificial Sequence
  synthetic peptide
LEGYFK

SEQ ID NO: 804 - protein, 6 aa, Artificial Sequence
  synthetic peptide
LESGFK

SEQ ID NO: 805 - protein, 6 aa, Artificial Sequence
  synthetic peptide
LGYEFK

SEQ ID NO: 806 - protein, 6 aa, Artificial Sequence
  synthetic peptide
LSGEFK

SEQ ID NO: 807 - protein, 6 aa, Artificial Sequence
  synthetic peptide
LGEFYK

SEQ ID NO: 808 - protein, 6 aa, Artificial Sequence
  synthetic peptide
LSEFGK

SEQ ID NO: 809 - protein, 6 aa, Artificial Sequence
  synthetic peptide
LQEGYN

SEQ ID NO: 810 - protein, 6 aa, Artificial Sequence
  synthetic peptide
LQESGN

SEQ ID NO: 811 - protein, 6 aa, Artificial Sequence
  synthetic peptide
LQGYEN

SEQ ID NO: 812 - protein, 6 aa, Artificial Sequence
  synthetic peptide
LQSGEN

SEQ ID NO: 813 - protein, 6 aa, Artificial Sequence
  synthetic peptide
LGYQEN

SEQ ID NO: 814 - protein, 6 aa, Artificial Sequence
  synthetic peptide
LSGQEN

SEQ ID NO: 815 - protein, 6 aa, Artificial Sequence
  synthetic peptide
LGQEYN

SEQ ID NO: 816 - protein, 6 aa, Artificial Sequence
  synthetic peptide
LSQEGN

SEQ ID NO: 817 - protein, 6 aa, Artificial Sequence
  synthetic peptide
LNQGYT

SEQ ID NO: 818 - protein, 6 aa, Artificial Sequence
  synthetic peptide
LNQSGT

SEQ ID NO: 819 - protein, 6 aa, Artificial Sequence
  synthetic peptide
LNGYQT

SEQ ID NO: 820 - protein, 6 aa, Artificial Sequence
  synthetic peptide
LNSGQT

SEQ ID NO: 821 - protein, 6 aa, Artificial Sequence
  synthetic peptide
LGYNQT

SEQ ID NO: 822 - protein, 6 aa, Artificial Sequence
  synthetic peptide
LSGNQT

SEQ ID NO: 823 - protein, 6 aa, Artificial Sequence
  synthetic peptide
LGNQYT

SEQ ID NO: 824 - protein, 6 aa, Artificial Sequence
  synthetic peptide
LSNQGT

SEQ ID NO: 825 - protein, 6 aa, Artificial Sequence
  synthetic peptide
LFHGYK

SEQ ID NO: 826 - protein, 6 aa, Artificial Sequence
  synthetic peptide
LFHSGK

SEQ ID NO: 827 - protein, 6 aa, Artificial Sequence
  synthetic peptide
LFGYHK

SEQ ID NO: 828 - protein, 6 aa, Artificial Sequence
  synthetic peptide
LFSGHK

SEQ ID NO: 829 - protein, 6 aa, Artificial Sequence
  synthetic peptide
LGYFHK

SEQ ID NO: 830 - protein, 6 aa, Artificial Sequence
  synthetic peptide
LSGFHK

SEQ ID NO: 831 - protein, 6 aa, Artificial Sequence
  synthetic peptide
LGFHYK

SEQ ID NO: 832 - protein, 6 aa, Artificial Sequence
  synthetic peptide
LSFHGK

SEQ ID NO: 833 - protein, 6 aa, Artificial Sequence
  synthetic peptide
LFKGYD

SEQ ID NO: 834 - protein, 6 aa, Artificial Sequence
  synthetic peptide
LFKSGD

SEQ ID NO: 835 - protein, 6 aa, Artificial Sequence
  synthetic peptide
LFGYKD

SEQ ID NO: 836 - protein, 6 aa, Artificial Sequence
  synthetic peptide
LFSGKD

SEQ ID NO: 837 - protein, 6 aa, Artificial Sequence
  synthetic peptide
LGYFKD

SEQ ID NO: 838 - protein, 6 aa, Artificial Sequence
  synthetic peptide
LSGFKD

SEQ ID NO: 839 - protein, 6 aa, Artificial Sequence
  synthetic peptide
LGFKYD

SEQ ID NO: 840 - protein, 6 aa, Artificial Sequence
  synthetic peptide
LSFKGD

SEQ ID NO: 841 - protein, 6 aa, Artificial Sequence
  synthetic peptide
LHDGYF

SEQ ID NO: 842 - protein, 6 aa, Artificial Sequence
  synthetic peptide
LHDSGF

SEQ ID NO: 843 - protein, 6 aa, Artificial Sequence
  synthetic peptide
LHGYDF

SEQ ID NO: 844 - protein, 6 aa, Artificial Sequence
  synthetic peptide
LHSGDF

SEQ ID NO: 845 - protein, 6 aa, Artificial Sequence
  synthetic peptide
LGYHDF

SEQ ID NO: 846 - protein, 6 aa, Artificial Sequence
  synthetic peptide
LSGHDF

SEQ ID NO: 847 - protein, 6 aa, Artificial Sequence
  synthetic peptide
LGHDYF

SEQ ID NO: 848 - protein, 6 aa, Artificial Sequence
  synthetic peptide
LSHDGF

SEQ ID NO: 849 - protein, 6 aa, Artificial Sequence
  synthetic peptide
LTDGYK

SEQ ID NO: 850 - protein, 6 aa, Artificial Sequence
  synthetic peptide
LTDSGK

SEQ ID NO: 851 - protein, 6 aa, Artificial Sequence
  synthetic peptide
LTGYDK

SEQ ID NO: 852 - protein, 6 aa, Artificial Sequence
  synthetic peptide
LTSGDK

SEQ ID NO: 853 - protein, 6 aa, Artificial Sequence
  synthetic peptide
LGYTDK

SEQ ID NO: 854 - protein, 6 aa, Artificial Sequence
  synthetic peptide
LSGTDK

SEQ ID NO: 855 - protein, 6 aa, Artificial Sequence
  synthetic peptide
LGTDYK

SEQ ID NO: 856 - protein, 6 aa, Artificial Sequence
  synthetic peptide
LSTDGK

SEQ ID NO: 857 - protein, 6 aa, Artificial Sequence
  synthetic peptide
LDEGYH

SEQ ID NO: 858 - protein, 6 aa, Artificial Sequence
  synthetic peptide
LDESGH

SEQ ID NO: 859 - protein, 6 aa, Artificial Sequence
  synthetic peptide
LDGYEH

SEQ ID NO: 860 - protein, 6 aa, Artificial Sequence
  synthetic peptide
LDSGEH

SEQ ID NO: 861 - protein, 6 aa, Artificial Sequence
  synthetic peptide
LGYDEH

SEQ ID NO: 862 - protein, 6 aa, Artificial Sequence
  synthetic peptide
LSGDEH

SEQ ID NO: 863 - protein, 6 aa, Artificial Sequence
  synthetic peptide
LGDEYH

SEQ ID NO: 864 - protein, 6 aa, Artificial Sequence
  synthetic peptide
LSDEGH

SEQ ID NO: 865 - protein, 6 aa, Artificial Sequence
  synthetic peptide
DEPGYK

SEQ ID NO: 866 - protein, 6 aa, Artificial Sequence
  synthetic peptide
DEPSGK

SEQ ID NO: 867 - protein, 6 aa, Artificial Sequence
  synthetic peptide
DEGYPK

SEQ ID NO: 868 - protein, 6 aa, Artificial Sequence
  synthetic peptide
DESGPK

SEQ ID NO: 869 - protein, 6 aa, Artificial Sequence
  synthetic peptide
DGYEPK

SEQ ID NO: 870 - protein, 6 aa, Artificial Sequence
  synthetic peptide
DSGEPK

SEQ ID NO: 871 - protein, 6 aa, Artificial Sequence
  synthetic peptide
DGEPYK

SEQ ID NO: 872 - protein, 6 aa, Artificial Sequence
  synthetic peptide
DSEPGK

SEQ ID NO: 873 - protein, 6 aa, Artificial Sequence
  synthetic peptide
DELGYT

SEQ ID NO: 874 - protein, 6 aa, Artificial Sequence
  synthetic peptide
DELSGT

SEQ ID NO: 875 - protein, 6 aa, Artificial Sequence
  synthetic peptide
DEGYLT

SEQ ID NO: 876 - protein, 6 aa, Artificial Sequence
  synthetic peptide
DESGLT

SEQ ID NO: 877 - protein, 6 aa, Artificial Sequence
  synthetic peptide
DGYELT

SEQ ID NO: 878 - protein, 6 aa, Artificial Sequence
  synthetic peptide
DSGELT

SEQ ID NO: 879 - protein, 6 aa, Artificial Sequence
  synthetic peptide
DGELYT

SEQ ID NO: 880 - protein, 6 aa, Artificial Sequence
  synthetic peptide
DSELGT

SEQ ID NO: 881 - protein, 6 aa, Artificial Sequence
  synthetic peptide
DNKGYQ

SEQ ID NO: 882 - protein, 6 aa, Artificial Sequence
  synthetic peptide
DNKSGQ

SEQ ID NO: 883 - protein, 6 aa, Artificial Sequence
  synthetic peptide
DNGYKQ

SEQ ID NO: 884 - protein, 6 aa, Artificial Sequence
  synthetic peptide
DNSGKQ

SEQ ID NO: 885 - protein, 6 aa, Artificial Sequence
  synthetic peptide
DGYNKQ

SEQ ID NO: 886 - protein, 6 aa, Artificial Sequence
  synthetic peptide
DSGNKQ

SEQ ID NO: 887 - protein, 6 aa, Artificial Sequence
  synthetic peptide
DGNKYQ

SEQ ID NO: 888 - protein, 6 aa, Artificial Sequence
  synthetic peptide
DSNKGQ

SEQ ID NO: 889 - protein, 6 aa, Artificial Sequence
  synthetic peptide
DTEGYQ

SEQ ID NO: 890 - protein, 6 aa, Artificial Sequence
  synthetic peptide
DTESGQ

SEQ ID NO: 891 - protein, 6 aa, Artificial Sequence
  synthetic peptide
DTGYEQ

SEQ ID NO: 892 - protein, 6 aa, Artificial Sequence
  synthetic peptide
DTSGEQ

SEQ ID NO: 893 - protein, 6 aa, Artificial Sequence
  synthetic peptide
DGYTEQ

SEQ ID NO: 894 - protein, 6 aa, Artificial Sequence
  synthetic peptide
DSGTEQ

SEQ ID NO: 895 - protein, 6 aa, Artificial Sequence
  synthetic peptide
DGTEYQ

SEQ ID NO: 896 - protein, 6 aa, Artificial Sequence
  synthetic peptide
DSTEGQ

SEQ ID NO: 897 - protein, 6 aa, Artificial Sequence
  synthetic peptide
DKHGYP

SEQ ID NO: 898 - protein, 6 aa, Artificial Sequence
  synthetic peptide
DKHSGP

SEQ ID NO: 899 - protein, 6 aa, Artificial Sequence
  synthetic peptide
DKGYHP

SEQ ID NO: 900 - protein, 6 aa, Artificial Sequence
  synthetic peptide
DKSGHP

SEQ ID NO: 901 - protein, 6 aa, Artificial Sequence
  synthetic peptide
DGYKHP

SEQ ID NO: 902 - protein, 6 aa, Artificial Sequence
  synthetic peptide
DSGKHP

SEQ ID NO: 903 - protein, 6 aa, Artificial Sequence
  synthetic peptide
DGKHYP

SEQ ID NO: 904 - protein, 6 aa, Artificial Sequence
  synthetic peptide
DSKHGP

SEQ ID NO: 905 - protein, 6 aa, Artificial Sequence
  synthetic peptide
DLTGYF

SEQ ID NO: 906 - protein, 6 aa, Artificial Sequence
  synthetic peptide
DLTSGF

SEQ ID NO: 907 - protein, 6 aa, Artificial Sequence
  synthetic peptide
DLGYTF

SEQ ID NO: 908 - protein, 6 aa, Artificial Sequence
  synthetic peptide
DLSGTF

SEQ ID NO: 909 - protein, 6 aa, Artificial Sequence
  synthetic peptide
DGYLTF

SEQ ID NO: 910 - protein, 6 aa, Artificial Sequence
  synthetic peptide
DSGLTF

SEQ ID NO: 911 - protein, 6 aa, Artificial Sequence
  synthetic peptide
DGLTYF